(19) United States
(12) Patent Application Publication
(10) Pub. No.: US 2025/0229015 A1
(43) Pub. Date: Jul. 17, 2025

(54) ENDOVASCULAR IMPLANTS AND DEVICES AND METHODS FOR CREATING A PERCUTANEOUS ARTERIOVENOUS FISTULA

(71) Applicant: Venova Medical, Inc., Los Gatos, CA (US)

(72) Inventor: Erik van der Burg, Los Gatos, CA (US)

(21) Appl. No.: 18/949,348

(22) Filed: Nov. 15, 2024

Related U.S. Application Data
(60) Provisional application No. 63/599,494, filed on Nov. 15, 2023.

(51) Int. Cl. A61M 1/36

(52) U.S. Cl. CPC A61M 1/3655

(57) ABSTRACT

Various systems, devices, and methods for endovascular implants and accurate placement thereof are disclosed. The implants include a proximal implant segment, a distal implant segment, connector struts connecting the proximal implant segment to the distal implant segment, and a side opening between the proximal implant segment and the distal implant segment. The implants can be used to create an arteriovenous fistula or connect one vessel of the body to another by placement of the proximal implant segment and the distal implant segment within the vessels to be connected. The implants can have an hourglass shape. The implants can include a continuous strut or ring at a distal edge of the proximal implant segment. Also disclosed are methods for accurate percutaneous placement of the implants disclosed, and a device for percutaneous delivery.

### INCORPORATION BY REFERENCE TO ANY PRIORITY APPLICATIONS

This application claims the benefit of priority under 35 U.S.C. § 119(e) to U.S. Provisional Patent Application No. 63/599,494, filed Nov. 15, 2023. The above-mentioned application is hereby incorporated by reference herein in its entirety. Any and all applications for which a foreign or domestic priority claim is identified in the Application Data Sheet as filed with the present application are hereby incorporated by reference under 37 CFR 1.57.

### BACKGROUND

### Field

Some aspects herein relate to endovascular implant systems, methods and devices which provide for accurate percutaneous placement in the vasculature, including for creation of an arteriovenous (AV) fistula in the arm for dialysis access.

### Description of the Related Art

There are numerous interventional endovascular procedures that have been developed and are performed which require the accurate placement of implants such as endovascular stents, filters and covered stents (stent-grafts), to name a few. These endovascular procedures treat conditions such as vascular occlusive disease, vascular aneurysmal disease and other abnormalities of the vasculature. They may also be used to treat hypertension, both portal vein hypertension and systemic hypertension by shunting blood flow from the hypertensive vasculature to the lower pressure venous system. Another possible treatment that could be performed through an endovascular procedure is the creation of an arteriovenous fistula by placing a vascular implant between a vein and artery to create vascular access for hemodialysis.

These endovascular implant procedures typically rely on expensive radiographic imaging, such as fluoroscopy, and significant skill of the operator to precisely position the catheter-based delivery system prior to deployment and delivery of the implant. These techniques require special procedure rooms, the requirement to wear lead based protective equipment and the injection of toxic contrast media into the patient which can cause undue stress on the renal system. Transdermal ultrasound imaging does not provide the needed image resolution to ensure accurate positioning during these procedures. Improved implants and procedures are needed.

Hemodialysis in particular may benefit from improved implants and methods. Hemodialysis is a life-saving treatment for kidney failure that uses a machine, called a dialyzer, to filter a patient's blood outside the body. Vascular access is required to remove and return blood during the procedure. During hemodialysis, blood from the patient will flow from one point of the access (e.g., from a needle pierced into an access vein), through a tube to the dialyzer where waste and extra fluid are filtered out, then back through a different tube to a separate point of the access (e.g., through another needle pierced into the same access vein or another) in order to return it to the patient. Vascular access allows large amounts of blood to flow continuously during hemodialysis treatment so that as much blood as possible can be filtered during the procedure. Vascular access generally consists of two types: long-term use which includes arteriovenous fistulas and arteriovenous grafts, and short-term use which includes a venous catheter.

An arteriovenous (AV) fistula for use in hemodialysis is generally a connection between an artery and a vein made by a vascular surgeon. In the creation of an AV fistula, the vascular surgeon will connect an artery of the patient to a vein of the patient. Placement of the AV fistula is generally in the forearm or upper arm, and it is desired to connect an artery (which is located within muscle near deep veins) to a superficial vein (which is located atop/external the muscle and closer to the surface) for ease of access. The AV fistula exposes the vein to increased pressure and blood flow, causing it to grow large and strong. An enlarged vein provides an easier and more reliable target for vascular access, increased blood flow allows for single vein access and more blood to be filtered, and increased strength enables the vein to handle the repeated needle insertions of serial treatments as well as prevents the vein from collapsing during the procedure.

An AV graft for use in hemodialysis is generally a looped, plastic tube implanted in the patient (e.g., it does not exit the skin) that connects an artery and a vein, installed surgically by a vascular surgeon. As opposed to a patient's vein being used for vascular access during hemodialysis, the AV graft is used for access to the vasculature (e.g., access needles are pierced through the graft tubing instead of a patient's vein).

A venous catheter for use in hemodialysis is a tube inserted into a vein in the patient's neck, chest, or leg near the groin, usually only for short-term hemodialysis due to the increased risk of sepsis and mortality by this approach. The tube splits in two after exiting the body to allow for the two connections typical of hemodialysis treatment (e.g., blood out, filtered blood in). If a patient's disease has progressed quickly, a patient may not have time for placement of an AV fistula or an AV graft before starting hemodialysis treatments, as both generally require 2-3 months to develop/mature before they can be used for hemodialysis; in this situation, a venous catheter may be required until longer-term vascular access is developed.

Among the ways to create access for hemodialysis, an AV fistula is preferred over the other types mentioned because it provides for good blood flow for dialysis, it lasts longer, and is less likely to get infected or cause blood clots than the other types of access. Although preferred, there remain drawbacks to the current practices of creating an AV fistula. One of the main drawbacks includes the requirement for a vascular surgeon to surgically create the AV fistula, which requires appropriate personnel, facilities and infrastructure to perform.

More recent methods for creating an AV fistula, such as by catheter electrocautery, may allow for a more non-invasive approach but they do not overcome all the drawbacks of the traditional surgical method and can introduce new drawbacks. Namely, due to the anatomical requirement that the AV fistula be created in adjacent vessels by a catheter electrocautery approach, an AV fistula will be created between an artery and a deep vein, not an artery and a superficial vein directly which is the desired type of access vein for hemodialysis. While perforating veins do extend between and connect deep veins to superficial veins, deep veins also have multiple branching points in the anatomical areas typically used for the creation of an AV fistula. Thus, an AV fistula created by a catheter electrocautery approach may disperse blood flow from the artery through multiple venous branches, and only a portion may be directed to a desired superficial vein which may not be enough to induce the required anatomical changes in the superficial vein as discussed above or provide the required blood flow for a hemodialysis treatment procedure. Secondary procedures such as band ligation and embolization of the connected branching veins may be required to direct blood from the artery to the desired superficial vein, which delay the availability of long-term vascular access for the patient and require extended access via a venous catheter, subjecting the patient to the increased risks of that access modality. There remains a need for improved methods, systems and devices for creating an AV fistula for hemodialysis.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods.

In some embodiments, disclosed herein is an implant configured for percutaneous delivery into an arm of a patient for the creation of an arteriovenous fistula. The implant can comprise: a proximal implant segment comprising a proximal end, a distal end, and an axis extending therethrough; a distal implant segment connected to the proximal implant segment, the distal implant segment comprising a proximal end, a distal end, and an axis extending therethrough; and a side opening between the distal end of the proximal implant segment and the proximal end of the distal implant segment; wherein the implant comprises an hourglass shape.

In the above implant or in other embodiments as described herein, one or more of the following features may also be provided. In some embodiments, the side opening is configured to allow blood to flow through the side opening (i) in a first direction along the proximal implant segment from the distal end of the proximal implant segment to the proximal end of the proximal implant segment, and (ii) in a second direction along the distal implant segment from the proximal end of the distal implant segment to the distal end of the distal implant segment.

In some embodiments, the implant is configured to allow blood to (i) flow through the side opening and in a first direction along the proximal implant segment from the distal end of the proximal implant segment to the proximal end of the proximal implant segment, (ii) flow through the side opening and in a second direction along the distal implant segment from the proximal end of the distal implant segment to the distal end of the distal implant segment, and (iii) flow in a third direction along the distal implant segment from the distal end of the distal implant segment to the proximal end of the distal implant segment.

In some embodiments, the side opening is configured to allow blood to flow through the side opening and (i) through the distal end of the proximal implant segment and out the proximal end of the proximal implant segment, and (ii) through the proximal end of the distal implant segment and out the distal end of the distal implant segment. In some embodiments, the implant is configured to allow blood to flow through the proximal end of the distal implant and out the distal end of the distal implant segment. In some embodiments, the implant is configured to allow blood to flow through the distal end of the distal implant and out the proximal end of the distal implant segment.

In some embodiments, the implant is configured to allow retrograde blood flow in the artery to (i) flow through the distal end of the distal implant segment and out the proximal end of the distal implant segment, and (ii) flow through the distal end of the proximal implant segment and out the proximal end of the proximal implant segment. In some embodiments, the implant is configured to allow antegrade blood flow and/or retrograde blood flow through the distal implant segment while blood flows through the distal end of the proximal implant segment and out the proximal end of the distal implant segment.

In some embodiments, the implant comprises an asymmetric hourglass shape. In some embodiments, the implant comprises a first longitudinal portion, a second longitudinal portion, a third longitudinal portion, a fourth longitudinal portion, and a fifth longitudinal portion, wherein: the proximal implant segment comprises, in order from its proximal end to its distal end, the first, second, third and fourth longitudinal portions; the distal implant segment comprises the fifth longitudinal portion; the first longitudinal portion has a first diameter; the third longitudinal portion has a second diameter; the fifth longitudinal portion has a third diameter; the second longitudinal portion has a diameter that transitions along its longitudinal length from the first diameter to the second diameter; the fourth longitudinal portion has a diameter that transitions along its longitudinal length from the second diameter to the third diameter; and the second diameter is smaller than the first diameter and the third diameter. In some embodiments, the third diameter is smaller than the first diameter.

In some embodiments, the side opening extends from at least a portion of the third longitudinal portion to at least a portion of the fifth longitudinal portion. In some embodiments, the proximal implant segment is capable of bending or curving relative to the distal implant segment by at least 180 degrees in a direction away from the side opening.

In some embodiments, the proximal implant segment comprises a series of rows of struts arranged substantially circumferentially about the axis of the proximal implant segment, said series of rows of struts of the proximal implant segment connected to one another by one or more axially extending struts arranged substantially along the axis of the proximal implant segment. In some embodiments, adjacent rows of struts of the proximal implant segment are connected by no more than one axially extending strut. In some embodiments, each row of struts comprises a row of wavy struts. In some embodiments, the distal implant segment comprises a series of rows of struts arranged at least partially circumferentially around the axis of the distal implant segment, said series of rows of struts of the distal implant segment connected to one another by one or more axially extending struts arranged substantially along the axis of the distal implant segment. In some embodiments, the series of rows of struts of the distal implant segment are connected to one another by at least one more axially extending strut than the series of rows of struts of the proximal implant segment to provide for greater flexibility of the proximal implant segment relative to the distal implant segment. In some embodiments, a majority or all of the one or more axially extending struts of the proximal implant segment are located on a side of the proximal implant segment aligned with a location of the side opening.

In some embodiments, the proximal implant segment has a greater flexibility than the distal implant segment. In some embodiments, the proximal implant segment comprises a tubular body with a flow lumen extending between its proximal end and its distal end. In some embodiments, the distal implant segment comprises an at least partially tubular body extending between its proximal end and its distal end. In some embodiments, the distal implant segment comprises a tubular body with a flow lumen extending between its proximal end and its distal end. In some embodiments, the proximal implant segment and the distal implant segment comprise expandable bodies, and wherein the proximal implant segment is oriented at an angle relative to the distal implant segment when the proximal and the distal implant segments are fully expanded in an at rest configuration. In some embodiments, an axial length of the proximal implant segment is greater than an axial length of the distal implant segment. In some embodiments, the distal end of the proximal implant segment comprises an anastomotic ring. In some embodiments, one or both of the proximal implant segment and the distal implant segment comprise one or more anchors configured to anchor said one or both segments against a wall of a vein or an artery.

In some embodiments, one or both of the proximal implant segment and the distal implant segment are covered with a graft material. In some embodiments, the implant is configured to collapse to a collapsed configuration for percutaneous delivery into the arm of the patient and to expand from the collapsed configuration to an expanded configuration for implantation between a vein and an artery of the patient. In some embodiments, the proximal implant segment and the distal implant segment are formed from a single unitary body. In some embodiments, the proximal implant segment and the distal implant segment comprise a metallic frame. In some embodiments, the proximal implant segment and the distal implant segment are formed from a single, laser cut hypotube. In some embodiments, the implant comprises one or more axially extending struts connecting the distal end of the proximal implant segment to the proximal end of the distal implant segment.

In some embodiments, disclosed herein is an implant configured for percutaneous delivery for the creation of an arteriovenous fistula. The implant can comprise: a proximal implant segment comprising a proximal end, a distal end, and an axis extending therethrough; a distal implant segment connected to the proximal implant segment, the distal implant segment comprising a proximal end, a distal end, and an axis extending therethrough; and a side opening between the distal end of the proximal implant segment and the proximal end of the distal implant segment; and a flow restrictor at or adjacent the side opening, the flow restrictor configured to limit blood flow through the proximal implant segment.

In the above implant or in other embodiments as described herein, one or more of the following features may also be provided. In some embodiments, the implant comprises a first longitudinal portion, a second longitudinal portion, a third longitudinal portion, a fourth longitudinal portion, and a fifth longitudinal portion, wherein: the proximal implant segment comprises, in order from its proximal end to its distal end, the first, second, third and fourth longitudinal portions; the distal implant segment comprises the fifth longitudinal portion; the first longitudinal portion has a first diameter; the third longitudinal portion has a second diameter; the fifth longitudinal portion has a third diameter; the second longitudinal portion has a diameter that transitions along its longitudinal length from the first diameter to the second diameter; the fourth longitudinal portion has a diameter that transitions along its longitudinal length from the second diameter to the third diameter; and the second diameter is smaller than the first diameter and the third diameter. In some embodiments, the third longitudinal portion comprises the flow restrictor.

In some embodiments, disclosed herein is a method of creating an arteriovenous fistula in a patient, comprising: delivering an intraluminal implant in a collapsed configuration into the patient, the intraluminal implant comprising: a proximal implant segment comprising a proximal end and a distal end; a distal implant segment comprising a proximal end and a distal end, wherein the proximal end of the distal implant segment is connected to the distal end of the proximal implant segment; and a side opening between the proximal implant segment and the distal implant segment; and radially expanding the intraluminal implant between a vein and an artery, wherein the proximal implant segment extends at least partially through the vein, the distal implant segment extends at least partially through the artery, to cause retrograde blood flow in the artery to (i) flow through the distal end of the distal implant segment and out the proximal end of the distal implant segment, and (ii) flow through the distal end of the proximal implant segment and out the proximal end of the proximal implant segment.

In the above method or in other embodiments as described herein, one or more of the following features may also be provided. In some embodiments, radially expanding the intraluminal implant between the vein and the artery causes antegrade blood flow in the artery to flow into the side opening and (i) through the proximal end of the distal implant segment and out the distal end of the distal implant segment to flow further into the artery, and (ii) through the distal end of the proximal implant segment and out the proximal end of the proximal implant segment to flow into the vein. In some embodiments, radially expanding the intraluminal implant between the vein and the artery causes antegrade and retrograde blood flow in the artery.

In some embodiments, disclosed herein is a method of creating an arteriovenous fistula in a patient, comprising: delivering an intraluminal implant in a collapsed configuration into the patient, the intraluminal implant comprising: a proximal implant segment comprising a proximal end and a distal end; a distal implant segment comprising a proximal end and a distal end, wherein the proximal end of the distal implant segment is connected to the distal end of the proximal implant segment; and a side opening between the proximal implant segment and the distal implant segment; and radially expanding the intraluminal implant between a vein and an artery, wherein the proximal implant segment extends at least partially through a cubital perforator vein and the distal implant segment extends at least partially through a radial artery, to cause blood flow in the radial artery to flow through the distal end of the proximal implant segment and out the proximal end of the proximal implant segment into a cephalic vein; wherein after radially expanding the intraluminal implant between the vein and the artery, a brachial artery flow rate is between about 600 ml/min and about 2000 ml/min.

In the above method or in other embodiments as described herein, one or more of the following features may also be provided. In some embodiments, radially expanding the implant between the vein and the artery causes antegrade flow in the radial artery to flow into the side opening. In some embodiments, radially expanding the implant between the vein and the artery causes retrograde flow in the radial artery to flow into the distal end of the distal implant segment. In some embodiments, radially expanding the implant between the vein and the artery causes a diameter of the cephalic vein to increase to at least about 6 mm in diameter. In some embodiments, after 6 weeks after radially expanding the implant between the vein and the artery, the brachial artery flow rate is greater than about 800 ml/min.

In some embodiments, disclosed herein is a method of creating an arteriovenous fistula in a patient, comprising: delivering an intraluminal implant in a collapsed configuration into the patient within a sheath constraining the intraluminal implant at a distal end of the sheath, the intraluminal implant comprising a proximal implant segment and a distal implant segment, wherein the proximal implant segment is connected to the distal implant segment, wherein a portion of the distal end of the sheath is delivered into the patient within a proximal cavity of a nose cone, and wherein the nose cone comprises an angled proximal end; extending the intraluminal implant within the sheath between a first vessel and a second vessel, wherein the proximal implant segment extends at least partially through the first vessel and the distal implant segment and nose cone are positioned within the second vessel and distal to the proximal implant segment; withdrawing the nose cone and sheath proximally such that the angled proximal end of the nose cone engages a wall of the second vessel; withdrawing the sheath proximally relative to the nose cone to (i) radially expand the distal implant segment within the proximal cavity of the nose cone, and (ii) radially expand the proximal implant segment and cause the proximal implant segment to engage a wall of the first vessel; and distally advancing the nose cone relative to the distal implant segment to further radially expand the distal implant segment and cause the distal implant segment to engage the wall of the second vessel.

In the above method or in other embodiments as described herein, one or more of the following features may also be provided. In some embodiments, the implant comprises an hourglass shape. In some embodiments, the intraluminal implant comprises a side opening between a distal end of the proximal implant segment and a proximal end of the distal implant segment, such that after the proximal implant segment is radially expanded to engage the wall of the first vessel and the distal implant segment is radially expanded to engage the wall of the second vessel, blood flowing through the artery enters the side opening and (i) flows through the proximal end of the distal implant segment and a distal end of the distal implant segment to continue through the second vessel, and (ii) flows through the distal end of the proximal implant segment and out a proximal end of the proximal implant segment to flow into the first vessel. In some embodiments, the implant is configured to allow retrograde blood flow in the artery to (i) flow through the distal end of the distal implant segment and out the proximal end of the distal implant segment, and (ii) flow through the distal end of the proximal implant segment and out the proximal end of the proximal implant segment. In some embodiments, the implant comprises an asymmetric hourglass shape. In some embodiments, the implant comprises a first longitudinal portion, a second longitudinal portion, a third longitudinal portion, a fourth longitudinal portion, and a fifth longitudinal portion, wherein: the proximal implant segment comprises, in order from its proximal end to its distal end, the first, second, third and fourth longitudinal portions; the distal implant segment comprises the fifth longitudinal portion; the first longitudinal portion has a first diameter; the third longitudinal portion has a second diameter; the fifth longitudinal portion has a third diameter; the second longitudinal portion has a diameter that transitions along its longitudinal length from the first diameter to the second diameter; the fourth longitudinal portion has a diameter that transitions along its longitudinal length from the second diameter to the third diameter; and the second diameter is smaller than the first diameter and the third diameter. In some embodiments, the third diameter is smaller than the first diameter. In some embodiments, the side opening extends from at least a portion of the third longitudinal portion to at least a portion of the fifth longitudinal portion.

In some embodiments, disclosed herein is a system for creating an arteriovenous fistula in an arm of a patient, the system comprising: an endovascular delivery device configured for access into the arm of the patient, wherein the endovascular delivery device is configured to be advanced into a superficial vein, into a perforator vein, into a deep vein, and into an artery adjacent to the deep vein; and an intraluminal implant, wherein the endovascular delivery device is configured to carry the intraluminal implant in a radially compressed configuration into the arm of the patient, the intraluminal implant comprising: a proximal implant segment comprising a proximal end and a distal end, the proximal implant segment being releasable from the endovascular delivery device to transform from a radially compressed configuration to a radially expanded configuration in which the proximal implant segment extends at least partially through the perforator vein and the deep vein with the proximal end of the proximal implant segment positioned within the perforator vein; and a distal implant segment connected to the proximal implant segment, the distal implant segment being releasable from the endovascular delivery device to transform from a radially compressed configuration to a radially expanded configuration in which the distal implant segment is positioned within the artery, wherein the distal end of the proximal implant segment is configured to be at an angle relative to an axis of the distal implant segment; wherein when the proximal implant segment is in the radially expanded configuration extending at least partially through the perforator vein and the deep vein and the distal implant segment is in the radially expanded configuration within the artery, the proximal implant segment is configured to divert flow from the artery into the superficial vein.

In the above system or in other embodiments as described herein, one or more of the following features may also be provided. In some embodiments, the distal implant segment is configured to anchor against a wall of the artery. In some embodiments, the distal implant segment comprises a tubular body configured to provide radial support to the artery. In some embodiments, the proximal implant segment comprises a tubular body configured to radially engage a wall of the perforator vein. In some embodiments, the distal end of the proximal implant segment is configured to be secured to a wall of the artery. In some embodiments, the distal end of the proximal implant segment comprises an anchor configured to anchor against the wall of the artery. In some embodiments, one or both of the proximal implant segment and the distal implant segment is covered with a graft material. In some embodiments, the implant comprises a side opening between the distal end of the proximal implant segment and a proximal end of the distal implant segment, wherein when the proximal implant segment is in the radially expanded configuration extending through the perforator vein and the deep vein and the distal implant segment is in the radially expanded configuration within the artery, blood flowing through the artery enters the side opening and (i) flows through the proximal end of the distal implant segment and out a distal end of the distal implant segment, and (ii) flows through the distal end of the proximal implant segment and out the proximal end of the proximal implant segment. In some embodiments, blood in the artery flows in a retrograde direction and (i) flows through the distal end of the distal implant segment and out the proximal end of the distal implant segment, and (ii) flows through the distal end of the proximal implant segment and out the proximal end of the proximal implant segment. In some embodiments, the distal end of the proximal implant segment and/or the proximal end of the distal implant comprises an anastomotic ring. In some embodiments, the distal end of the proximal implant segment is configured to be angled relative to an axis of the distal implant segment by between about 0 degrees to about 180 degrees. In some embodiments, the distal implant segment is connected to the proximal implant segment by at least one connecting strut. In some embodiments, the implant comprises an hourglass shape. In some embodiments, the implant comprises an asymmetric hourglass shape. In some embodiments, the delivery device comprises a sheath configured to constrain the intraluminal implant in a radially compressed configuration within a distal end of the sheath. In some embodiments, the delivery device further comprises a nose cone advanceable into the artery, and wherein the distal end of the sheath is configured to be inserted within a cavity of the nose cone for advancement with the nose cone into the artery. In some embodiments, the nose cone comprises an angled proximal end configured to engage a near wall of the artery. In some embodiments, the delivery device is configured such that, after the distal end of the sheath is advanced with the nose cone into the artery: the sheath is retractable in a proximal direction relative to the nose cone to expand the proximal implant segment within the deep vein and the perforator vein; and the nose cone is distally advanceable relative to the distal implant segment after the proximal implant segment is expanded within the deep vein and the perforator vein to expand the distal implant segment within the artery. In some embodiments, the delivery device is configured such that, after the distal implant segment is expanded within the artery, the sheath is advanceable through the expanded proximal implant segment and the expanded distal implant segment into engagement with the nose cone to facilitate removal of the nose cone with the sheath from the artery. In some embodiments, the delivery device further comprises a guidewire shaft configured to be advanced over a guidewire, wherein the nose cone is fixed to the guidewire shaft.

In some embodiments, disclosed herein is a method of creating an arteriovenous fistula in an arm of a patient, comprising: delivering an intraluminal implant in a collapsed configuration into the patient, the intraluminal implant comprising a proximal implant segment and a distal implant segment, wherein the proximal implant segment is connected to the distal implant segment; extending the intraluminal implant between a deep vein and an artery adjacent the deep vein, wherein the proximal implant segment extends at least partially through a perforator vein and/or at least partially through the deep vein and the distal implant segment is positioned within the artery; and radially expanding the proximal implant segment to cause the proximal implant segment to engage a wall of the perforator vein and radially expanding the distal implant segment to cause the distal implant segment to engage a wall of the artery and provide radial support for the artery, such that blood flowing through the artery is diverted from the artery into a superficial vein connected to the perforator vein.

In the above method or in other embodiments as described herein, one or more of the following features may also be provided. In some embodiments, the intraluminal implant comprises a side opening between a distal end of the proximal implant segment and a proximal end of the distal implant segment, such that after the proximal implant segment is radially expanded to engage the wall of the perforator vein and the distal implant segment is radially expanded to engage the wall of the artery, blood flowing through the artery enters the side opening and (i) flows through the proximal end of the distal implant segment and a distal end of the distal implant segment to continue through the artery, and (ii) flows through the distal end of the proximal implant segment and out a proximal end of the proximal implant segment to flow into the perforator vein and into the superficial vein. In some embodiments, blood in the artery flows in a retrograde direction and (i) flows through the distal end of the distal implant segment and out the proximal end of the distal implant segment, and (ii) flows through the distal end of the proximal implant segment and out the proximal end of the proximal implant segment to flow into the perforator vein and into the superficial vein. In some embodiments, the proximal implant segment and the distal implant segment comprise tubular bodies. In some embodiments, the method further comprises anchoring a distal end of the proximal implant segment to the wall of the artery. In some embodiments, after the proximal implant segment is radially expanded to engage the wall of the perforator vein and the distal implant segment is radially expanded to engage the wall of the artery, the proximal implant segment is angled relative to an axis of the distal implant segment. In some embodiments, the proximal implant segment is angled relative to an axis of the distal implant segment by between about 0 degrees to about 180 degrees. In some embodiments, the intraluminal implant is delivered into the patient within a sheath constraining the intraluminal implant at a distal end of the sheath. In some embodiments, the distal end of the sheath is advanced into the artery within a cavity of a nose cone. In some embodiments, the proximal implant segment is released from the sheath to radially expand into engagement with the wall of the perforator vein by proximally retracting the sheath relative to the nose cone. In some embodiments, the distal implant segment is radially expanded into engagement with the wall of the artery by distally advancing the nose cone relative to the distal implant segment. In some embodiments, the method further comprises distally advancing the sheath through the radially expanded proximal implant segment and the radially expanded distal implant segment into engagement with the nose cone, and proximally retracting the sheath engaged with the nose cone through the radially expanded proximal implant segment and the radially expanded distal implant segment. In some embodiments, the nose cone comprises an angled proximal end that engages with a wall of the artery while the sheath is proximally retracted to release the proximal implant segment. In some embodiments, the nose cone is rotated within the artery after the nose cone has been distally advanced to release the distal implant segment and before proximally retracting the sheath engaged with the nose cone through the radially expanded proximal implant segment and the radially expanded distal implant segment.

In some embodiments, disclosed herein is a method of creating an arterio-venous fistula, comprising: accessing a superficial vein; advancing an access tool into the superficial vein, into a perforator vein, and into a deep vein; advancing the access tool through a luminal wall of the deep vein, through an or any interstitial space, and through an adventitial wall of an artery (also sometimes referred to herein as a “deep artery”); advancing a guidewire through the access tool into the artery; withdrawing the access tool over the guidewire; and/or advancing a device (also referred to herein as a “delivery device”) over the guidewire such that a distal end of the device is within the artery and a more proximal segment of the device spans the or any interstitial space.

In the above method or in other embodiments as described herein, one or more of the following features may also be provided. In some embodiments, the method can include, wherein advancing the access tool through the luminal wall of the deep vein, through the or any interstitial space, and through the adventitial wall of an artery comprises actuating a port proximate the proximate end of the access tool, thereby causing a sharpened needle tip to extend distally from a distal end of the access tool. In some embodiments, actuating a port comprises depressing the port and compressing a spring element operably connected to the sharpened needle tip. In some embodiments, the method further comprises releasing the port, thereby allowing the spring element to recoil and cause the sharpened needle tip to retract proximally into the distal end of the access tool. In some embodiments, the device or delivery device comprises a nose cone. In some embodiments, the nose cone comprises a proximal tapered end, a central lumen, a distal tapered end, and a longitudinal axis. In some embodiments, the device or delivery device comprises a flexible sheath comprising a longitudinal axis. In some embodiments, an implant is carried within the flexible sheath in a radially compressed configuration. In some embodiments, after advancing the device or delivery device over the guidewire, a distal end of the flexible sheath resides within the central lumen of the nose cone, and a gap is formed between the proximal tapered end of the nose cone and a sidewall of the flexible sheath as it enters the central lumen of the nose cone at the proximal tapered end of the nose cone, and wherein the longitudinal axis of the flexible sheath is not coaxial with the longitudinal axis of the nose cone. In some embodiments, the length of the gap is between about 5% and about 50% of a diameter of the proximal tapered end of the nose cone. In some embodiments, no gap between the proximal tapered end of the nose cone and a sidewall of the flexible sheath is formed and/or required. In some embodiments, the method further comprises withdrawing the nose cone and the flexible sheath proximally such that the nose cone engages a near wall of the artery. In some embodiments, the method further comprises withdrawing the sheath proximally, thereby allowing a proximal segment of the implant to transform from the radially compressed configuration to a radially expanded configuration. In some embodiments, withdrawing the sheath proximally releases an anchor that engages the proximal segment of the implant with respect to the near wall of the artery. In some embodiments, a distal segment of the implant remains within the nose cone in a radially compressed configuration while the proximal segment of the implant is in the radially expanded configuration. In some embodiments, the method further comprises advancing the nose cone with respect to the distal segment of the implant, thereby transforming the distal segment of the implant to a radially expanded configuration. In some embodiments, advancing the nose cone releases an anchor that engages the proximal segment of the implant with respect to the near wall of the artery. In some embodiments, withdrawing the sheath proximally releases an anchor that engages the proximal segment of the implant with respect to the near wall of the artery. In some embodiments, the method further comprises advancing the sheath distally through the distal segment of the implant in the radially enlarged configuration, thereby engaging the nose cone. In some embodiments, the method further comprises rotating the nose cone around its longitudinal axis. In some embodiments, the method further comprises withdrawing the nose cone and the flexible sheath proximally out of the artery, the or any interstitial space, the deep vein, the perforator vein, and the superficial vein, leaving the implant in place.

In some embodiments, disclosed herein is a method of creating a fistula, comprising: advancing an access tool through a luminal wall of a first lumen, through an or any interstitial space, and through an outer wall of a second lumen; advancing a guidewire through the access tool into the second lumen; withdrawing the access tool over the guidewire; and/or advancing a device (also referred to herein as a “delivery device”) over the guidewire such that a distal end of the device is within the second lumen and a more proximal segment of the device spans the or any interstitial space, wherein the device comprises a nose cone comprising a proximal tapered end, a central lumen, a distal tapered end, and a longitudinal axis, and a flexible sheath comprising a longitudinal axis, wherein after advancing the device over the guidewire, a distal end of the flexible sheath resides within the central lumen of the nose cone, and a gap is formed between the proximal tapered end of the nose cone and a sidewall of the flexible sheath as it enters the central lumen of the nose cone at the proximal tapered end of the nose cone, and wherein the longitudinal axis of the flexible sheath is not coaxial with the longitudinal axis of the nose cone.

In the above method or in other embodiments as described herein, one or more of the following features may also be provided. In some embodiments, the length of the gap is between about 5% and about 50% of a diameter of the proximal tapered end of the nose cone. In some embodiments, the gap is formed at least partially by deflecting the nose cone with respect the flexible sheath. In some embodiments, no gap between the proximal tapered end of the nose cone and a sidewall of the flexible sheath is formed and/or required. In some embodiments, deflecting the nose cone comprises actuating at least one pullwire. In some embodiments, the method further comprises withdrawing the nose cone and the flexible sheath proximally such that the nose cone engages a near wall of the second lumen. In some embodiments, an implant is carried within the flexible sheath in a radially compressed configuration. In some embodiments, the method further comprises withdrawing the sheath proximally, thereby allowing a proximal segment of the implant to transform from the radially compressed configuration to a radially expanded configuration. In some embodiments, withdrawing the sheath proximally releases an anchor that engages the proximal segment of the implant with respect to the near wall of the second lumen. In some embodiments, a distal segment of the implant remains within the nose cone in a radially compressed configuration while the proximal segment of the implant is in the radially expanded configuration. In some embodiments, the method further comprises advancing the nose cone with respect to the distal segment of the implant, thereby transforming the distal segment of the implant to a radially expanded configuration. In some embodiments, advancing the nose cone releases an anchor that engages the proximal segment of the implant with respect to the near wall of the second lumen.

In some embodiments, disclosed herein is an intraluminal implant, comprising: a proximal implant segment, a distal implant segment, and at least one axially-oriented connecting strut connecting the proximal implant segment and the distal implant segment, the proximal implant segment and the distal implant segment comprising a flow lumen therethrough, wherein the at least one axially-oriented connecting strut serves as the only connection between the proximal implant segment and the distal implant segment, wherein an axial length of the proximal implant segment is greater than an axial length of the distal implant segment, wherein the implant comprises a shape memory material.

In the above implant or in other embodiments as described herein, one or more of the following features may also be provided. In some embodiments, the implant is configured such that the distal implant segment comprises a diameter different than a diameter of the proximal implant segment when the implant is in an unstressed state. In some embodiments, the implant is configured such that the distal implant segment comprises a diameter smaller than a diameter of the proximal implant segment when the implant is in an unstressed state. In some embodiments, the implant is configured such that the distal implant segment comprises a perimeter different than a perimeter of the proximal implant segment when the implant is in an unstressed state. In some embodiments, the implant is configured such that the distal implant segment comprises a perimeter smaller than a perimeter of the proximal implant segment when the implant is in an unstressed state. In some embodiments, the implant is configured such that the distal implant segment comprises a cross-sectional area different than a cross-sectional area of the proximal implant segment when the implant is in an unstressed state. In some embodiments, the implant is configured such that the distal implant segment comprises a cross-sectional area smaller than a cross-sectional area of the proximal implant segment when the implant is in an unstressed state. In some embodiments, the implant is configured such that the proximal implant segment comprises a variable diameter and/or cross-sectional area when the implant is in an unstressed state. In some embodiments, the implant comprises an hourglass shape. In some embodiments, the implant comprises an asymmetric hourglass shape. In some embodiments, the implant is configured such that a distal end of the proximal implant segment and/or a proximal end of the distal implant segment comprises a continuous strut and/or ring. In some embodiments, the implant is configured such that a distal end of the proximal implant segment and/or a proximal end of the distal implant segment comprises a continuous strut and/or ring with one or more anchors. In some embodiments, the implant is configured such that the proximal implant segment comprises struts of uniform lengths. In some embodiments, the implant is configured such that the proximal implant segment comprises struts of variable lengths and/or variable widths. In some embodiments, the implant is configured such that the proximal implant segment comprises struts with lengths different than lengths of struts of the distal implant segment. In some embodiments, the implant is configured such that the distal implant segment is longitudinally offset from the proximal implant segment when the implant is in an unstressed state. In some embodiments, the proximal implant segment comprises a biocompatible graft material. In some embodiments, the distal implant segment comprises a biodegradable graft material. In some embodiments, the implant comprises a porous or non-porous laminating layer. In some embodiments, the implant comprises a coating comprising heparin and/or a therapeutic agent. In some embodiments, the implant comprises no more than two axially-oriented connecting struts connecting the proximal implant segment and the distal implant segment.

In some embodiments, disclosed herein is an intraluminal implant for creating an arteriovenous fistula, comprising: a proximal implant segment comprising a proximal end and a distal end, the proximal implant segment configured to extend at least partially through a perforator vein and/or at least partially through a deep vein with the proximal end of the proximal implant segment configured to be positioned at least partially within the perforator vein; and a distal implant segment connected to the proximal implant segment and configured to be positioned within an artery adjacent to the deep vein, wherein the distal end of the proximal implant segment is configured to be angled relative to an axis of the distal implant segment; wherein the proximal implant segment is configured to divert flow from the artery into a superficial vein connected to the perforator vein.

In the above implant or in other embodiments as described herein, one or more of the following features may also be provided. In some embodiments, the proximal implant segment and the distal implant segment comprise expandable tubular bodies. In some embodiments, the intraluminal implant comprises a side opening between the distal end of the proximal implant segment and a proximal end of the distal implant segment, such that blood flowing through the artery enters the side opening and (i) flows through the proximal end of the distal implant segment and out a distal end of the distal implant segment to continue through the artery, and (ii) flows through the distal end of the proximal implant segment and out the proximal end of the proximal implant segment to flow into the perforator vein and into the superficial vein. In some embodiments, blood in the artery flows in a retrograde direction and (i) flows through the distal end of the distal implant segment and out the proximal end of the distal implant segment, and (ii) flows through the distal end of the proximal implant segment and out the proximal end of the proximal implant segment to flow into the perforator vein and into the superficial vein. In some embodiments, the proximal implant segment is angled relative to an axis of the distal implant segment by between about 0 to about 180 degrees.

In some embodiments, disclosed herein is an intraluminal implant for creating an arterio-venous fistula, comprising: a venous implant segment comprising a first expandable tubular body having a first end and a second end and a lumen extending therethrough, wherein the first expandable tubular body is configured to be collapsed for delivery into a patient and is expandable to radially engage an inner wall of a vein; and an arterial implant segment comprising a second expandable tubular body having a first end and a second end and a lumen extending therethrough, wherein the second expandable tubular body is configured to be collapsed for delivery into the patient and is expandable to radially engage an inner wall of an artery located adjacent to the vein; wherein the second end of the venous implant segment is connected to the first end of the arterial implant segment to allow for the arterial implant segment to be angled relative to the venous implant segment when the venous implant segment and the arterial implant segment are in expanded configurations, and wherein angling of the arterial implant segment relative to the venous implant segment increases a distance between the second end of the venous implant segment and the first end of the arterial implant segment along one side of the implant to provide a side opening into the implant; and wherein when the venous implant segment radially engages the inner wall of the vein and the arterial implant segment radially engages the inner wall of the artery adjacent to the vein, blood flowing through the artery enters the side opening and (i) flows through the first end of the arterial implant segment and out the second end of the arterial implant segment, and (ii) flows through the second end of the venous implant and out the first end of the venous implant segment. In some embodiments, blood in the artery flows in a retrograde direction and (i) flows through the second end of the arterial implant segment and out the first end of the arterial implant segment, and (ii) flows through the second end of the venous implant segment and out the first end of the venous implant segment.

In some embodiments, disclosed herein is a delivery device for delivering a vascular implant between a vein and an artery, comprising: an outer sheath configured to constrain the implant in a low-profile configuration at a distal end of the outer sheath; and a nose cone comprising a proximal end and a distal end and a cavity, wherein the distal end of the outer sheath is insertable into the cavity for advancement of the nose cone and the distal end of the outer sheath through the vein and into the artery; wherein the outer sheath is retractable in a proximal direction relative to the nose cone to expand a distal segment of the implant within the cavity; wherein the outer sheath is further retractable in a proximal direction relative to the nose cone to expand a proximal segment of the implant within the vein; and wherein the nose cone is distally advanceable relative to the distal segment of the implant after the proximal segment is expanded within the vein to release the distal segment of the implant from the cavity within the artery.

In the above device or in other embodiments as described herein, one or more of the following features may also be provided. In some embodiments, the distal end of the nose cone is tapered. In some embodiments, the proximal end of the nose cone is tapered. In some embodiments, the proximal end of the nose cone is at an angle relative to a longitudinal length of the nose cone. In some embodiments, an angled proximal end of the nose cone is configured to engage a near wall of the artery after the nose cone is advanced into the artery. In some embodiments, the distal end of the outer sheath is advanceable through the distal segment of the implant and into the cavity after the release of the distal segment of the implant within the artery. In some embodiments, the distal end of the outer sheath is advanceable through the distal segment of the implant after the release of the distal segment of the implant within the artery to engage the proximal end of the nose cone, such that the nose cone enters the distal end of the outer sheath. In some embodiments, the delivery device further comprises a guidewire shaft configured to be advanced over a guidewire, wherein the nose cone is fixed to the guidewire shaft. In some embodiments, the delivery device further comprises a control knob connected to a proximal end of the outer sheath configured to retract and/or advance the outer sheath upon proximal and/or distal movement of the control knob, the control knob at least partially disposed within a handle of the delivery device. In some embodiments, the control knob is configured to releasably lock into a proximal most and/or a distal most position within the handle. In some embodiments, the delivery device further comprises a middle shaft within the outer sheath configured to prevent the implant from slipping proximally during retraction of the outer sheath. In some embodiments, a distal end of the middle shaft leads the distal end of the outer sheath when the outer sheath is advanced into the cavity. In some embodiments, the delivery device further comprises a middle shaft connector disposed within the handle and connected to a proximal end of the middle shaft, the middle shaft connector configured to engage with the control knob and cause the middle shaft to advance with the outer sheath when the outer sheath is advanced into the cavity. In some embodiments, the implant is constrained within the distal end of the outer sheath.

In some embodiments, disclosed herein is a method of creating an arteriovenous fistula between an artery and a vein of a patient, comprising: delivering an intraluminal implant in a collapsed configuration into the patient, the intraluminal implant comprising a venous implant segment comprising a first tubular body and an arterial implant segment comprising a second tubular body, wherein the venous implant segment is connected to the arterial implant segment; extending the intraluminal implant across any interstitial space between the artery and the vein; and radially expanding the venous implant segment to radially engage the vein and radially expanding the arterial implant segment to radially engage the artery; wherein when the venous and arterial implant segments are radially engaged with the vein and the artery, respectively, the arterial implant segment is angled relative to the venous implant segment to provide a side opening into the intraluminal implant that allows blood flowing through the artery to enter the side opening and (i) flow through the second tubular body of the arterial implant segment to continue through the artery, and (ii) flow through the first tubular body of the venous implant segment to flow into the vein. In some embodiments, blood flows in the artery in a retrograde direction and (i) flows through the second tubular body of the arterial implant segment and (ii) flows through the first tubular body of the venous implant segment to flow into the vein.

In some embodiments, disclosed herein is an intraluminal implant comprising an implant frame. The implant frame may comprise struts. The implant frame may be covered by an inner layer of porous graft material, such as ePTFE, and an outer layer of porous graft material, such as ePTFE. The inner and outer layers may be fused to encapsulate the implant frame. A thermoplastic laminating layer may be placed between the inner and outer layers of porous graft material to facilitate bonding of the inner and outer layers. The thermoplastic laminating layer may be placed over an outer surface of the implant frame, and the outer layer of porous graft material may be applied over the thermoplastic laminating layer.

The thermoplastic laminating layer may be non-porous. In some embodiments, the thermoplastic laminating layer may comprise a strip that is helically wrapped between the inner and outer layers. The thermoplastic laminating layer may comprise fluorinated ethylene propylene (FEP), polyethylene (PE), or thermoplastic polyurethane (TPU) film. In some embodiments, the thermoplastic laminating layer may be wrapped to leave gaps between each wrap of the thermoplastic laminating layer. In embodiments comprising a proximal implant segment and a distal implant segment as described elsewhere herein, the gaps may be provided along at least a proximal portion of the proximal implant segment to provide additional flexibility to the proximal implant segment. In some embodiments, no gaps are provided along a distal portion of the proximal implant segment, and/or no gaps are provided along the distal implant segment.

In some embodiments, disclosed herein is a method, system or device comprising, consisting essentially of, consisting of, and/or not comprising any number of features of the disclosure.

In some embodiments, disclosed herein is an intraluminal implant comprising one or more features of the foregoing summary or as summarized and/or described further herein.

In some embodiments, disclosed herein is a delivery system for delivering an intraluminal implant comprising one or more features of the foregoing summary or as summarized and/or described further herein.

In some embodiments, disclosed herein is a method for delivering an intraluminal implant comprising one or more features of the foregoing summary or as summarized and/or described further herein.

Any of the embodiments summarized above or as described further in the Detailed Description may further or alternatively comprise features of the systems, devices or methods summarized below.

### BRIEF DESCRIPTION OF THE DRAWINGS

The foregoing and other features, aspects, and advantages of the embodiments of the systems, devices, and methods described herein are described in detail below with reference to the drawings of various embodiments, which are intended to illustrate and not to limit the embodiments of the invention. The drawings comprise the following figures in which:

FIG. 1 depicts a simplified representation of a portion of the vasculature of the human arm indicating a potential area to create an anastomotic connection according to some embodiments.

FIGS. 2A-2D depict a method of percutaneously introducing an endovascular guidewire according to some embodiments.

FIGS. 3-15A depict methods of percutaneously implanting an endovascular implant according to some embodiments.

FIGS. 15B-15D depict various hemodynamics after implanting an endovascular implant according to some embodiments.

FIGS. 16A-16C depict an endovascular implant according to some embodiments.

FIG. 16D depicts a lamination of an implant frame according to some embodiments.

FIGS. 17A-17I depict an endovascular implant according to some embodiments.

FIGS. 18A-18D depict endovascular implants according to some embodiments.

FIGS. 19A-19B depict an endovascular implant according to some embodiments.

FIGS. 19C-19E depict an endovascular implant according to some embodiments.

FIG. 19F shows a plot of clinical results after implantation of an endovascular implant according to some embodiments.

FIGS. 20A-20C depict an endovascular implant according to some embodiments.

FIG. 21 depicts a partial cross-sectional view of various elements of a delivery system according to some embodiments.

FIGS. 22A-22B depict various views of nose cones of a delivery system according to some embodiments.

FIG. 23A depicts a perspective view of a handle of a delivery system according to some embodiments.

FIG. 23B depict a cross-sectional view of a handle of a delivery system according to some embodiments.

FIG. 24A depicts a perspective view of a delivery system according to some embodiments.

FIG. 24B depicts a cross-sectional perspective view of a delivery system according to some embodiments.

FIG. 24C depicts an exploded perspective view of a delivery system according to some embodiments.

FIG. 24D depicts a cross-sectional view of a distal end of a delivery system according to some embodiments.

FIG. 24E depicts a perspective view of a delivery system according to some embodiments with part of a handle housing of the delivery system removed from view.

FIG. 24F depicts a side view of a distal end of a delivery system according to some embodiments.

FIG. 24G depicts a perspective view of a delivery system carrying an implant in its radially compressed configuration according to some embodiments.

FIG. 24H depicts an enlarged view of the distal end of the delivery system of FIG. 24G, including an implant in its radially compressed configuration according to some embodiments.

FIG. 25 depicts a simplified representation of a portion of the human vasculature indicating a potential area to create an anastomotic connection according to some embodiments.

FIGS. 26-29 depict a method of percutaneously introducing an endovascular guidewire according to some embodiments.

FIGS. 30-40 depict a method of percutaneously implanting an endovascular implant according to some embodiments.

FIG. 41 depicts a method of bypassing a section of an artery with endovascular implants according to some embodiments.

FIG. 42 depicts a counter force mechanism and a method of using the same according to some embodiments.

Throughout the drawings, unless otherwise noted, reference numbers may be re-used to indicate a general correspondence between referenced elements. The drawings are provided to illustrate example embodiments described herein and are not intended to limit the scope of the disclosure.

### DETAILED DESCRIPTION

Embodiments disclosed herein relate generally to medical devices and methods. More particularly, some embodiments relate to endovascular implants and methods and devices to efficiently and accurately place them in the vasculature. In some embodiments the devices, methods and systems described herein allow for the precise placement of devices such as stents, including covered stents, and other implants and anastomotic devices for the creation of arteriovenous fistulas (AVF) while minimizing or eliminating the need for radiographic imaging, thus allowing them to be performed in a clinical setting with only the use of non-invasive imaging techniques, such as transdermal ultrasound (e.g., a high resolution ultrasound system, e.g., a high frequency linear transducer (about 9-15 MHz)). Some embodiments utilize novel means for temporarily engaging anatomical structures while delivering endovascular implants. Furthermore, in some embodiments the implants, devices, systems, and/or methods described herein advantageously overcome some or all of the drawbacks of existing implants, devices, systems, and/or methods for the creation of an AVF, including the bypassing of deep vein branching that may undesirably divert arterial blood flow away from a desired superficial vein, and the prevention or reduction of secondary procedures such as ligation and embolization.

Any and/or all of the implementations and/or features of the endovascular implants, methods, devices, and/or systems described and/or illustrated herein can be applied to the various implants, methods, devices, and/or systems described and/or illustrated in U.S. Pat. No. 11,491,272, titled “ENDOVASCULAR IMPLANTS AND DEVICES AND METHODS FOR ACCURATE PLACEMENT”, and International Patent Publication No. WO 2023/220232, titled “ENDOVASCULAR IMPLANTS AND DEVICES AND METHODS FOR CREATING A PERCUTANEOUS ARTERIOVENOUS FISTULA”, the entire contents of which are hereby incorporated by reference in their entirety and for all purposes, and vice versa.

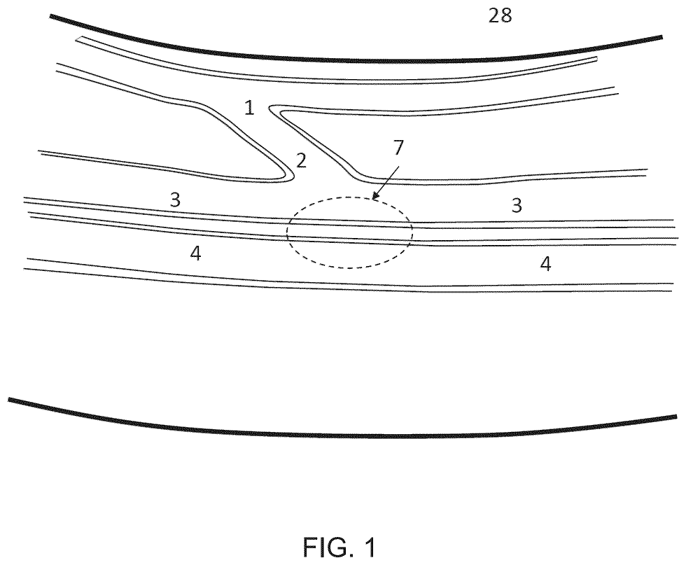

FIG. 1 depicts a simplified representation of a portion of the vasculature of the human arm with skin surface, e.g., dermal surface 28. Location 7 is a potential area to create an anastomotic connection between a first body lumen (which can also be referred to herein as a “first vessel”) and a second body lumen (which can also be referred to herein as a “second vessel”), such as, for example, an artery and a vein, such as an AVF between deep vein 3 and adjacent deep artery 4. Perforator vein 2 connects the superficial vein 1 to the deep vein 3 which lies adjacent and provides a conduit for accessing location 7. An AVF may be made between perforator vein 2 and artery 4, bypassing deep vein 3. In some embodiments, an AVF between perforator vein 2 and artery 4 may divert blood from the artery into superficial vein 1 connected to the perforator vein 2.

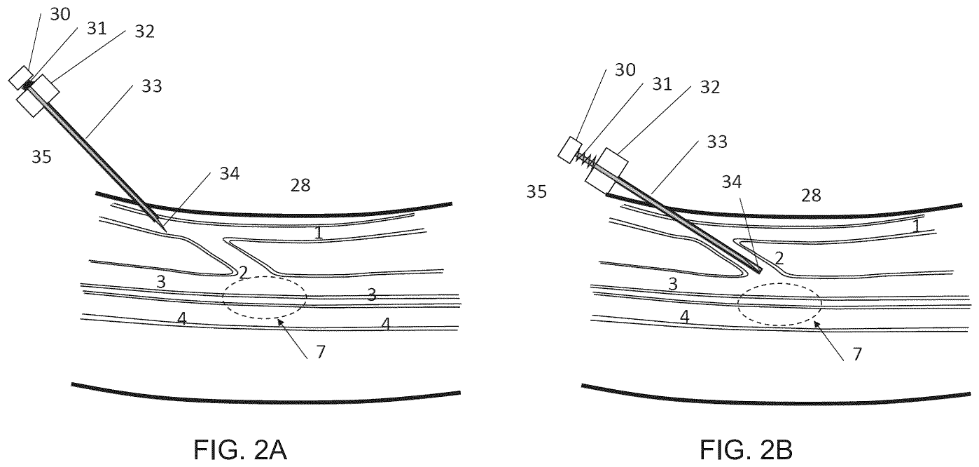

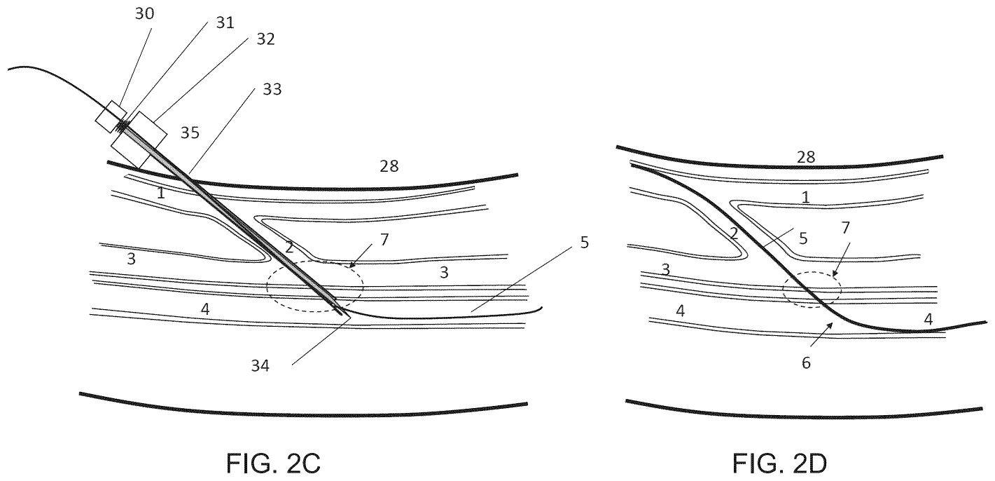

FIGS. 2A, 2B, 2C and 2D depict some embodiments of a method to percutaneously introduce endovascular guidewire 5 into deep artery 4 using needle access tool 35. One example of the endovascular guidewire 5 can have a diameter of about 0.018 inches and a length of about 80 cm. One example of a suitable guidewire 5 is a Nitrex® (Medtronic) 5 cm angled tip #N180802. Needle access tool 35 can include a hollow needle with proximal port 30 and distal sharpened tip 34 slidably disposed within sheath 33. The hollow needle can be an echogenic needle, such as an echogenic 21 g needle. The hollow needle can have a length of about 7 cm. One example of a suitable needle is Cook Micropuncture® set #G43869. Sheath 33 is connected to hub 32 that has compression element, such as compression spring 31 placed between port 30 and hub 32. When port 30 is depressed, needle tip 34 is exposed distally of the distal end of sheath 33 and able to puncture tissue such as skin and blood vessels. When port 30 is not depressed, spring 31 will decompress and move needle tip 34 proximally so that it is not exposed. In this configuration, the needle access device can navigate the vasculature with reduced risk of inadvertent punctures and trauma to the vasculature. Using this feature of the needle access tool 35 and appropriate imaging techniques, such as transdermal ultrasound, the needle access tool is first introduced into superficial vein 1 as shown in FIG. 2A. With needle tip 34 retracted within sheath 33, the needle access tool is navigated to location 7 using appropriate imaging as shown in FIG. 2B. While at location 7, the proximal port 30 is actuated (e.g., depressed) to expose needle tip 34 and the needle access tool 35 is then advanced to penetrate the vascular walls and any interstitial tissues between deep vein 3 and deep artery 4 until the distal end of sheath 33 enters the lumen of deep artery 4. While maintaining this position, guidewire 5 is introduced into proximal port 30 and advanced through the needle access tool 35 until the distal end of guidewire 5 exits the distal end of the needle access tool 35 and enters the lumen of deep artery 4 as shown in FIG. 2C. FIG. 2D shows guidewire 5 with curvature 6 which forms when guidewire 5 conforms to the particular vascular anatomy. Needle access tool 35 can have alternative embodiments which may include curved distal ends to allow for easier navigation through the vasculature, hemostasis valves attached to the proximal port 30 and a spring-loaded hollow needle that can aid in puncturing mobile structures. Alternative sites might also be chosen to location 7 to create the AVF. For example, a location more distal along deep vein 3 may prove more advantageous if the distance between deep vein 3 and deep artery 4 is less than at location 7. Some embodiments are not limited to connections between deep veins and deep arteries, such as in the upper extremities or the lower extremities, for example, such as in the hand, forearm, arm, foot, calf, thigh, or other areas. Some embodiments may be used to precisely locate implants in other luminal structures such as superficial veins and superficial arteries, coronary arteries, gynecological structures (e.g., the vagina, cervix, uterus, or fallopian tubes), urological structures (e.g., the ureters, bladder, or urethra) and gastrointestinal structures (e.g., esophagus, stomach, small intestine, large intestine, rectum, biliary tree, and others for example).

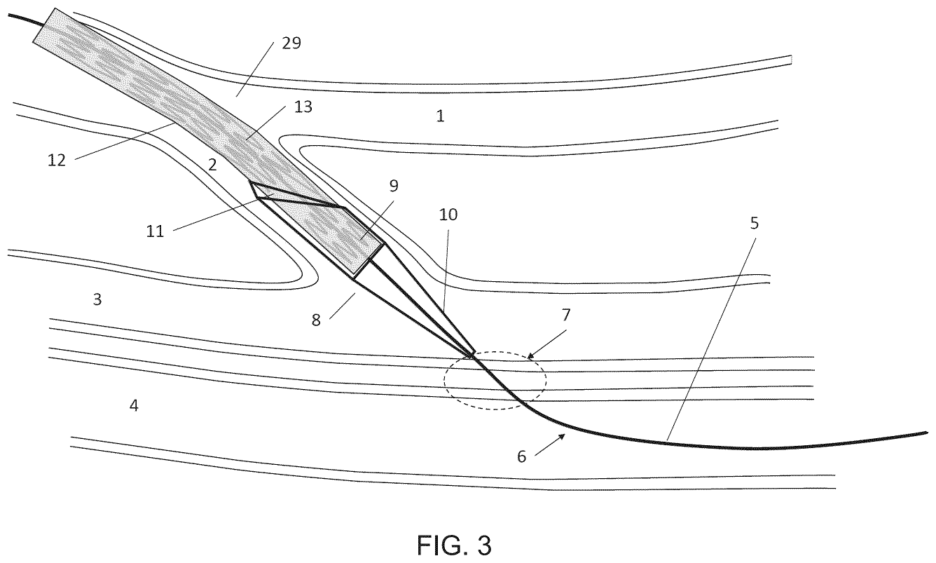

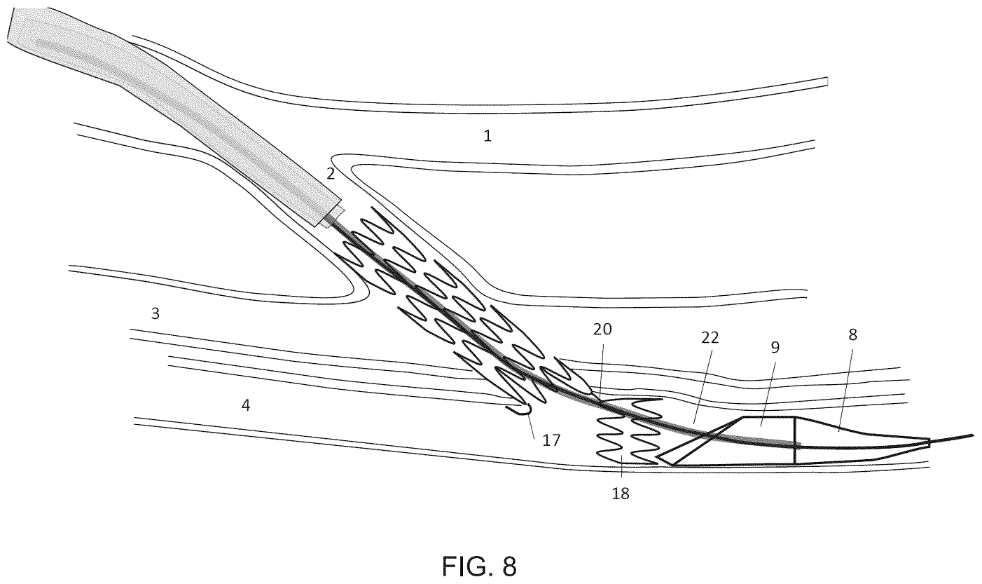

FIG. 3 depicts the distal end of an endovascular delivery system 29 with a distal nose cone 8 that has a distal taper 10, a cavity 9 (e.g., a central lumen) and angled proximal end 11 being introduced into the vascular system and approaching AVF location 7 over guidewire 5 with a curvature 6. An outer sheath 12 is shown constraining an implant 13 in a low-profile (e.g., collapsed) configuration with the distal end of outer sheath 12 inserted into cavity 9. Also shown, at least a portion of the implant 13 can be positioned within the cavity 9 of the nose cone 8. Delivery system 29 is shown with a bend conforming to guidewire 5 and the shape of the vascular anatomy. Delivery system 29 can be flexible or relatively stiff compared to the surrounding vascular structures and guidewire 5. Nose cone 8 has as a distal taper 10 so that it can more easily penetrate the vascular walls and any interstitial tissues at AVF location 7. In one embodiment, the very distal end of the distal taper 10 may have a sharpened point to further facilitate penetration of the various tissues. The angled proximal end of the nose cone 8 can be at an angle relative to a longitudinal length of the nose cone 8 to define an inclined opening into the cavity 9. Nose cone 8 has features to accommodate guidewire 5 and in this embodiment is bonded or otherwise fixed to an inner guide wire shaft 22 as shown in FIG. 8, but not shown in FIG. 3.

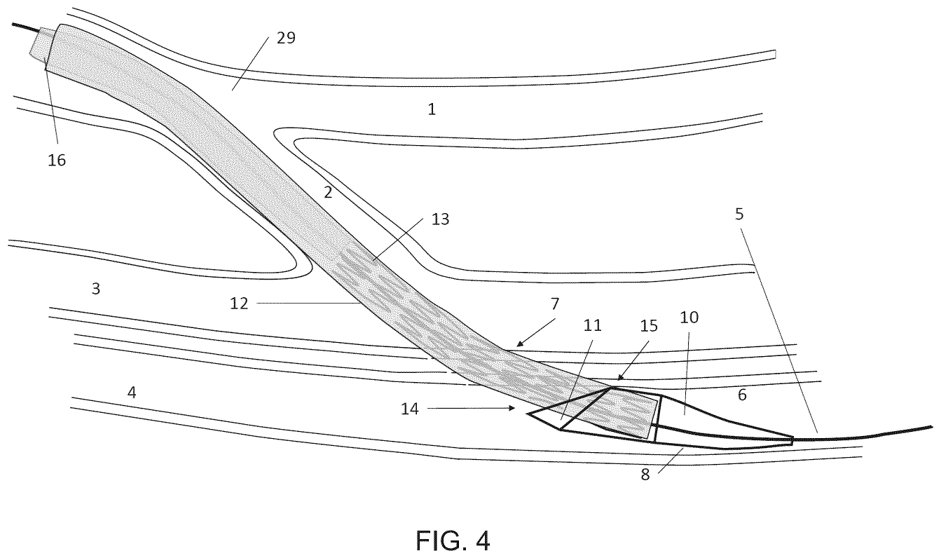

FIG. 4 depicts the nose cone 8 of delivery system 29 advanced across AVF location 7 and within deep artery 4. Middle shaft 16 is shown slidably disposed within outer sheath 12. Middle shaft 16 is slidably disposed around guidewire shaft 22 which is not shown in FIG. 4. Also depicted is gap 14 which may form when the delivery system 29 follows guidewire curvature 6 and the nose cone 8 and outer sheath 12 are no-longer coaxial. The gap 14 can be defined in some embodiments as an open space between the proximal opening of the nose cone 8 and a sidewall of the outer sheath 12 as it enters the proximal opening of the nose cone 8. In some embodiments, the gap defines about, at least about, or no more than about 5%, 10%, 15%, 20%, 25%, 30%, 35%, 40%, 45%, 50%, or more or less in length and/or diameter of the respective length and/or diameter of the proximal opening of the nose cone 8, or ranges including any two of the foregoing values. In some embodiments, no gap between the proximal opening of the nose cone 8 and a sidewall of the outer sheath 12 is formed and/or required.

Angle 15 between the central (e.g., longitudinal) axis of nose cone 8 and the central (e.g., longitudinal) axis of outer sheath 12 may form when the distal end of delivery system 29 is in a curved configuration where the proximal end of angled proximal end 11 is on the outside of the curve as shown in FIG. 4. In some embodiments, the angle 15 can be, for example, about, at least about, or no more than about 5, 10, 15, 20, 25, 30, 35, 40, 45 degrees, or more or less, and ranges including any two of the foregoing values. To facilitate greater flexibility between nose cone 8 and outer sheath 12, nose cone 8 may have a slit in the wall that forms cavity 9 (e.g. a slit extending distally from the angled proximal end 11 along at least a portion of the longitudinal length of the nose cone 8). In some embodiments, the curvature of guidewire 5 may be used to form angle 15 and thus gap 14. Alternative means of forming angle 15 and gap 14 can be utilized. An alternative embodiment could be, for example, to use one, two, or more pull wires to deflect the distal end of delivery system 29 so that gap 14 is formed. A wide variety of steerable and/or deflectable elements can be utilized depending on the desired clinical result. In some cases, a gap 14 may be formed by a difference between an outer diameter of the outer sheath 12 and an inner diameter of the cavity 9 of the nose cone 8.

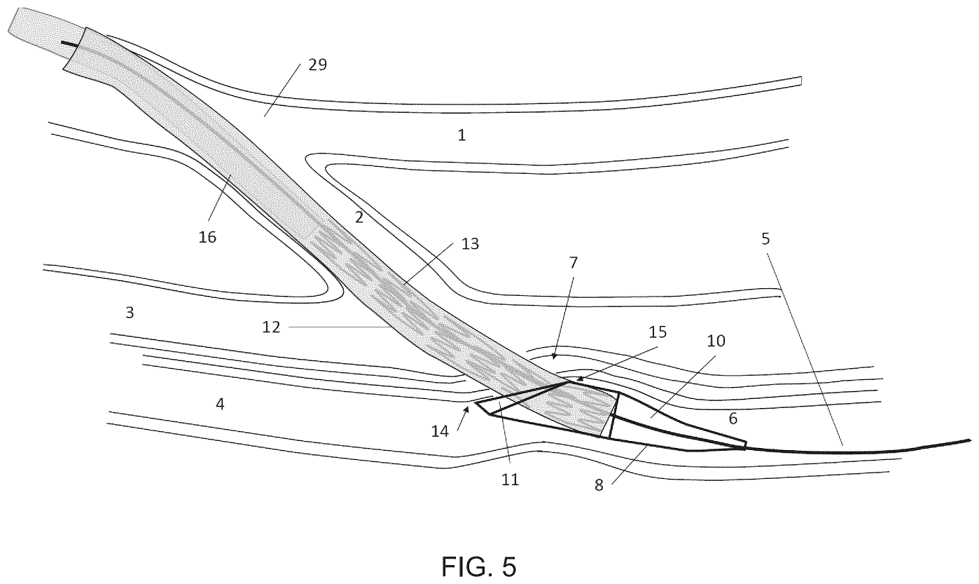

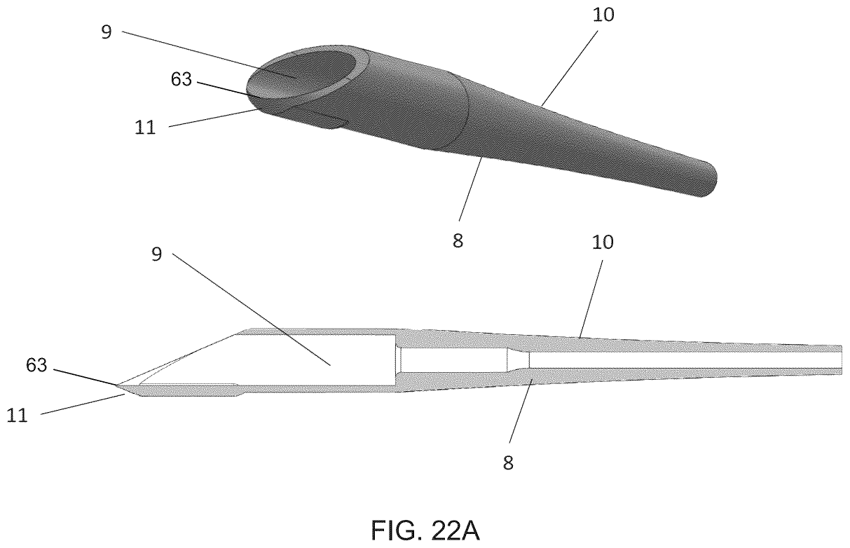

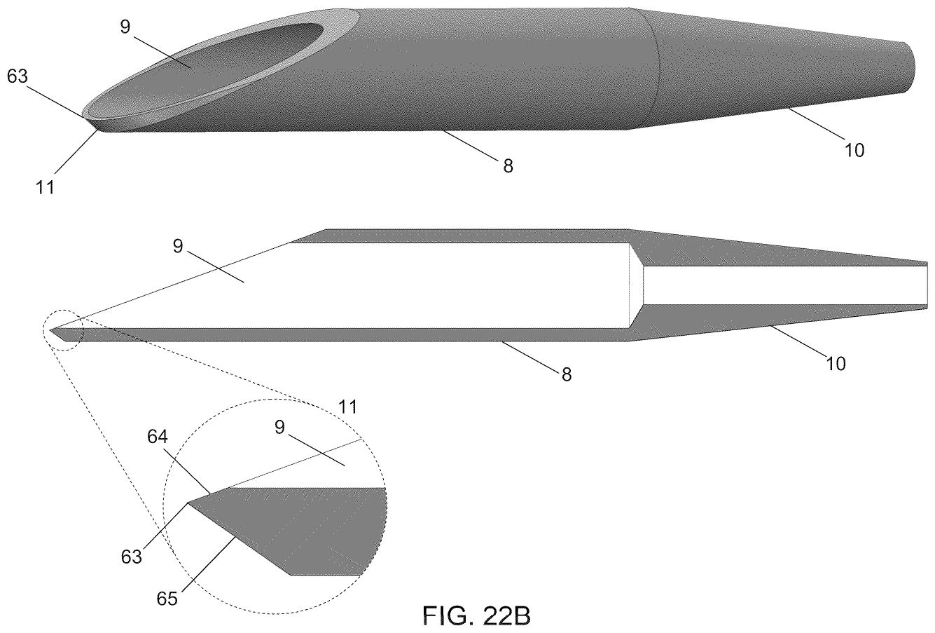

FIG. 5 depicts nose cone 8 engaging the near wall of deep artery 4 after delivery system 29 has been pulled proximally from its location in FIG. 4. The engagement between nose cone 8 and the near wall of deep artery 4 can be due to gap 14 which can be formed when delivery system 29 was urged into a curved configuration with the proximal end (e.g., the proximal-most end) of angled proximal end 11 on the outside of the curvature. In some embodiments, the nose cone 8 can engage the near wall of the artery without a gap 14 required. For example, the proximal-most end of angled proximal end 11 may engage the near wall of the artery 4 without a gap 14 between the angled proximal end 11 and the outer sheath 12, by, e.g., using a first surface 64 (shown in FIG. 22B) to engage the near wall of deep artery 4. Also depicted is the deformation of the anatomy at AVF location 7 which is a result of the apposition forces between the near wall of deep artery 4 and angled proximal end 11 of nose cone 8. This deformation of the anatomy at AVF location 7 may be visualized by ultrasound and used to verify proper tissue engagement and/or implant placement before delivery. One way to visualize this engagement and ensure that the nose cone 8 is oriented properly during deployment of the implant 13 by ultrasound is by using a long axis view that includes both the perforator vein 2 along its length and the deep artery 4.

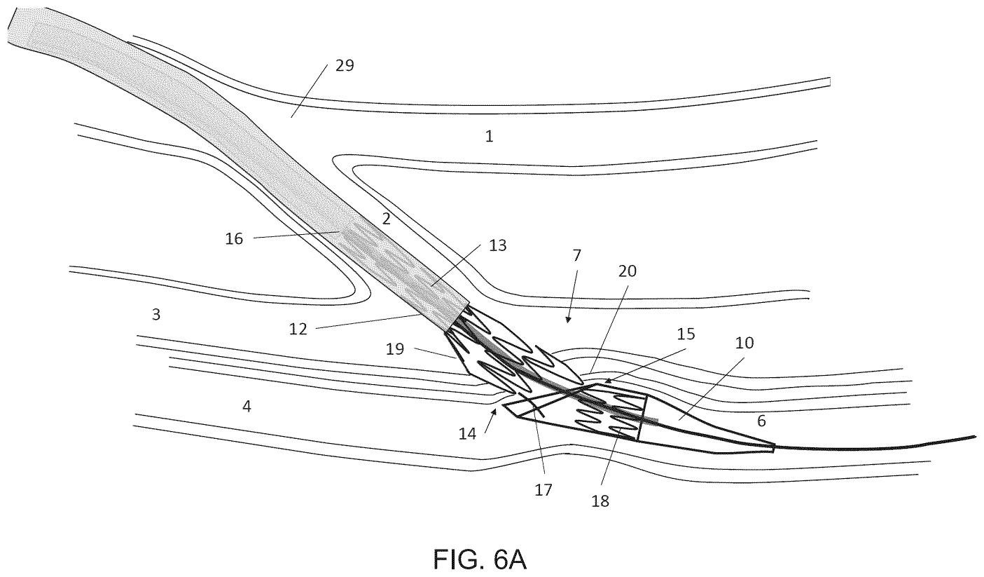

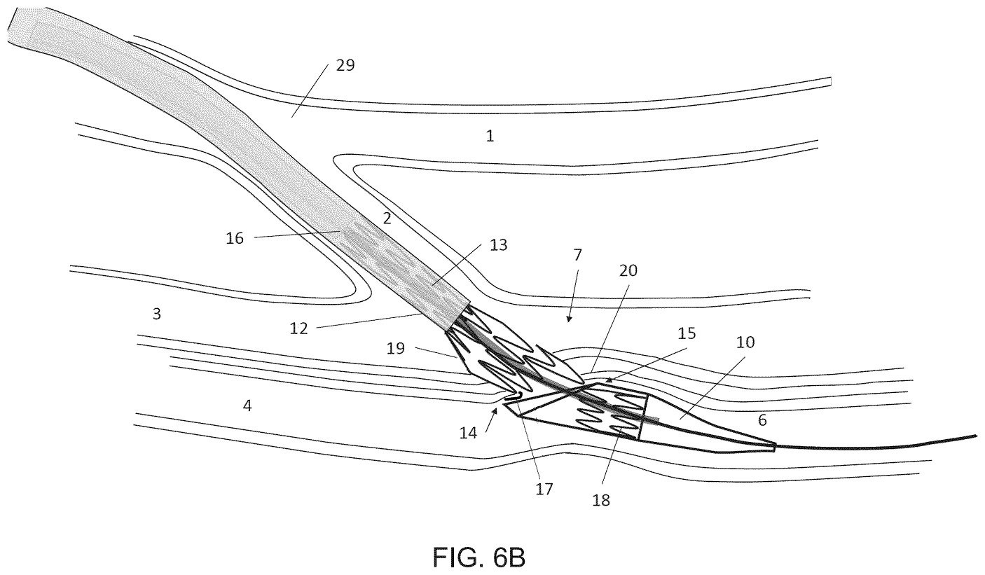

FIG. 6A depicts an initial step of the first stage of delivery of elastically constrained (e.g., collapsed) implant 13, according to some embodiments. While nose cone 8 is held in apposition against the near wall of deep artery 4, outer sheath 12 is retracted proximally so that the elastically constrained (e.g., collapsed) implant 13 is allowed to expand with the precise location defined due to the engagement between nose cone 8 and the near wall of deep artery 4. The distal end of middle shaft 16 is held fixed during delivery so that elastically constrained (e.g., collapsed) implant 13 does not slip proximally during the retraction of outer sheath 12. Also depicted is distal implant segment 18 being held partially constrained (e.g., partially collapsed) by cavity 9. Connector struts 20 that can be, for example, axially oriented as illustrated connect distal implant segment 18 to proximal implant segment 19 which has not yet been fully released (e.g., expanded) in FIG. 6A. Also partially constrained (e.g., partially collapsed) in cavity 9 is one or more anchors 17. Depicted in FIG. 6A is the radial expansion at AVF location 7 due to the radial stiffness of the elastically expanding implant 13. The distal end of proximal implant segment 19 may be angled with respect to an axis of the distal implant segment 18 of the implant so that it does not obstruct deep artery 4 while still fully within and supporting the area between the vascular walls of deep vein 3 and deep artery 4. In some embodiments, the distal end of proximal implant segment 19 may be at an angle of between about 0 degrees to about 90 degrees with respect to the axis of the distal implant segment 18.

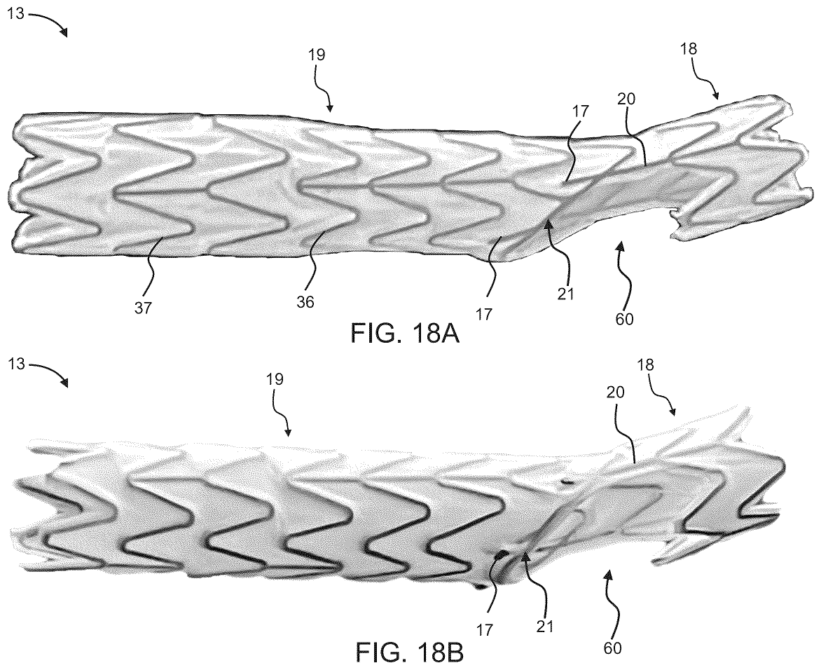

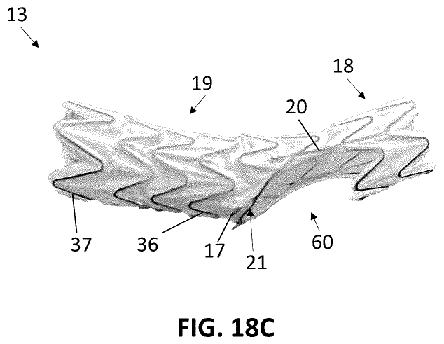

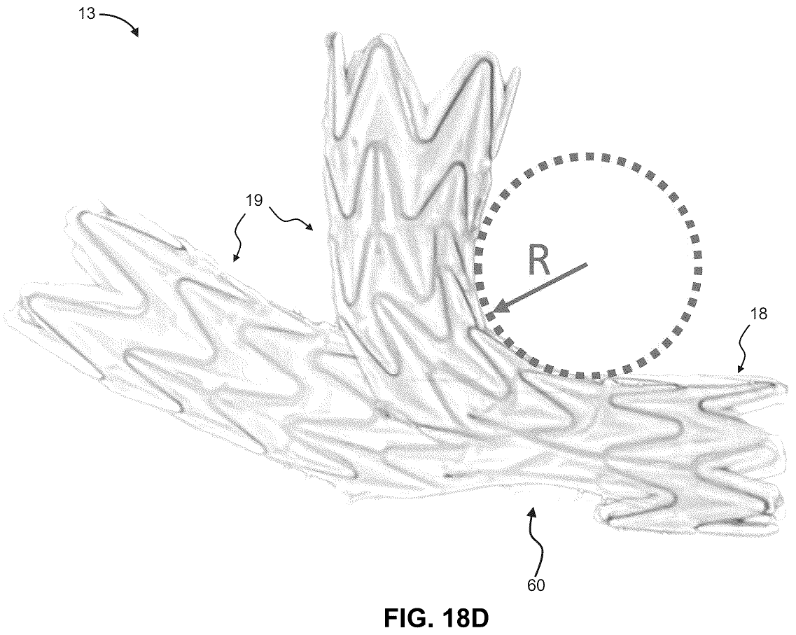

In some embodiments, and as shown in FIG. 6B, retraction of outer sheath 12 proximally may release one or more anchors 17. Anchor(s) 17 may engage the near wall of deep artery 4 (as shown in FIG. 6B), a wall of deep vein 3, a wall of perforator vein 2, a wall of superficial vein 1, and/or the or any interstitial tissues. In some embodiments, anchors 17 may include proximal anchors that extend proximally after being unconstrained (e.g., expanded or released). In one example, one or more anchors 17 may extend from the distal end of the proximal implant segment 19 in a distal direction while being constrained by the outer sheath 12 or nose cone 8, as described below. After the outer sheath 12 is retracted, the anchors 17 may move to an expanded (e.g., unconstrained or released) configuration in which the anchors extend radially outwardly and curve or bend in a proximal direction to facilitate engagement with a vessel wall or interstitial tissue. In other examples, such as shown in FIGS. 18A-18C, anchors may extend in a proximal direction while constrained by the outer sheath 12 or nose cone 8 and when unconstrained.

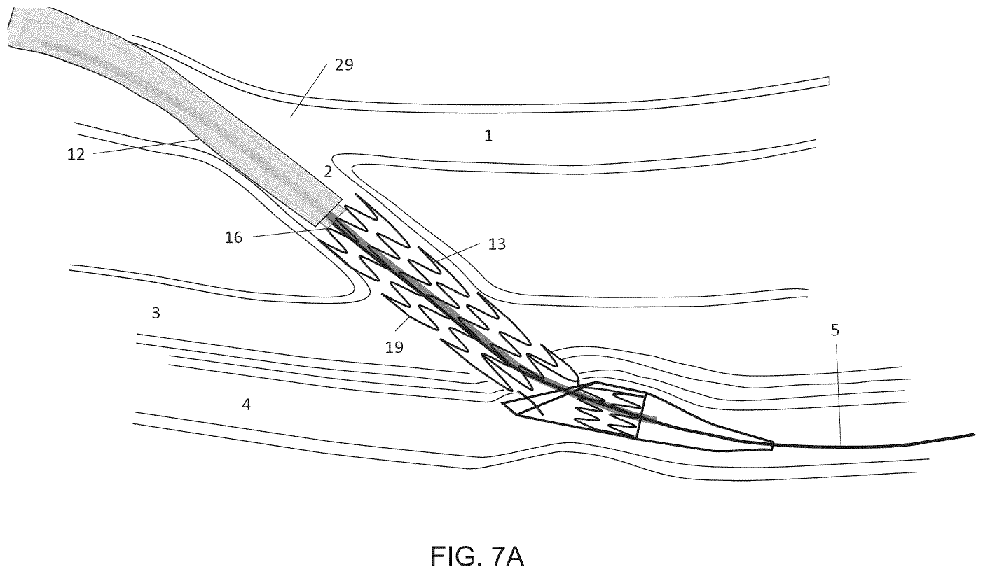

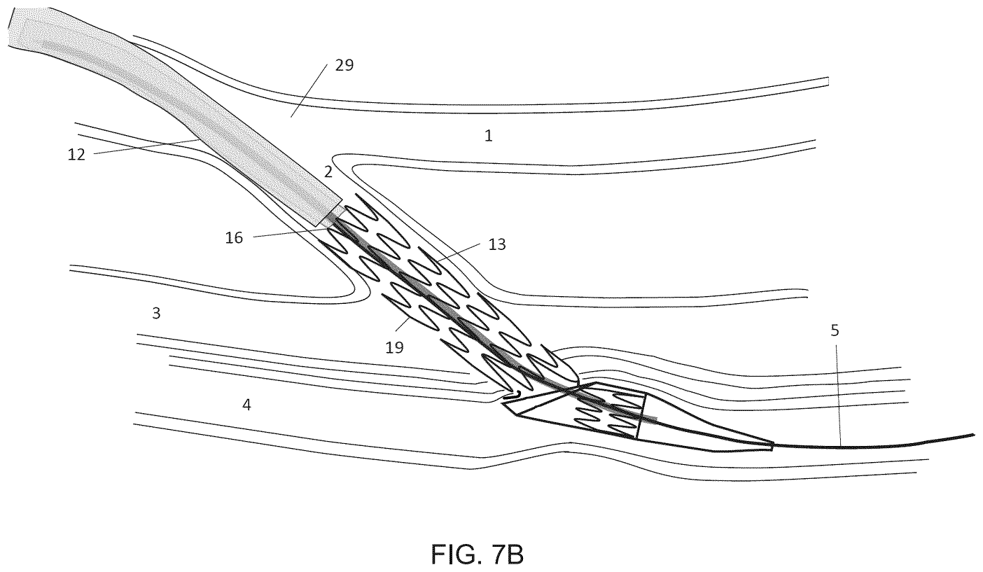

FIG. 7A follows from FIG. 6A and depicts continuing delivery (e.g., expansion) of elastically constrained (e.g., collapsed) implant 13 with the further retraction of outer sheath 12 until proximal implant segment 19 is fully released (e.g., expanded) from outer sheath 12. FIG. 7B follows from FIG. 6B and depicts continuing delivery (e.g., expansion) of elastically constrained (e.g., collapsed) implant 13 with the further retraction of outer sheath 12 until proximal implant segment 19 is fully released (e.g., expanded) from outer sheath 12.

FIG. 8 depicts the delivery of distal implant segment 18 of implant 13 and, in some embodiments, the release of anchor(s) 17. When nose cone 8 is advanced distally by advancing guidewire shaft 22 distally, distal implant segment 18 is held in its axial position by connector struts 20 and thus is slidably released from cavity 9. In some embodiments, anchor(s) 17, which may be shaped into, for example, a hook configuration, may also be elastically released from cavity 9 with advancement of nose cone 8 and assume their hook shape to secure the most proximal portion of the distal edge of proximal implant segment 19 to the near wall of deep artery 4. In some embodiments, anchor(s) 17 may be released upon retraction of outer sheath 12 proximally and upon nose cone 8 advancement distally. In some embodiments, anchor(s) 17 extend generally proximally when unconstrained. In some embodiments, anchor(s) 17 have a generally linear or curvilinear configuration. In alternative embodiments, there are no anchor(s) 17 due to sufficient anchoring provided by the apposition between the struts of implant 13 and the surrounding anatomy.

In the embodiment shown, distal implant segment 18 provides a means of securing the most distal portion of the distal edge of proximal implant segment 19 so that it does not encroach into the luminal space of deep artery 4 or substantially block the lumen of deep artery 4. Distal implant segment 18 can also provide radial support for deep artery 4 to ensure patency and sufficient distal blood flow after implantation of the implant 13. Distal implant segment 18 can be sized to be accommodated by the deep artery 4. In some embodiments, the diameter of distal implant segment 18 is 0-50% larger than the deep artery 4. In other embodiments, the diameter of distal implant segment 18 is 5-25% larger than the deep artery 4. In some embodiments, the proximal implant segment 19 has a diameter of between about 2 mm to about 7 mm. In some embodiments, the distal implant segment 18 has a diameter of between about 2 mm to about 7 mm. In some embodiments, the proximal implant segment 19 and the distal implant segment 18 may have about the same diameter. In some embodiments, the proximal implant segment 19 and the distal implant segment 18 may have different diameters. In some embodiments, the proximal implant segment 19 has a diameter of about 5 mm and the distal implant segment 18 has a diameter of about 4 mm. In some embodiments, the distal implant segment 18 has a diameter of about 4 mm, and the proximal implant segment 19 is tapered such that the distal end of the proximal implant segment 19 has a diameter of about 4 mm and the proximal end of the proximal implant segment 19 has a diameter of about 5 mm. In some embodiments, the proximal implant segment 19 and/or the distal implant segment 18 may not be circular in cross-sectional shape, and thus they may instead have cross-sectional areas and/or perimeters that can be the same or different. In some embodiments, there is no distal implant segment 18 portion of implant 13. In some embodiments, there is no distal implant segment 18 portion of implant 13 and the distal end of proximal implant segment 19 may comprise a continuous strut and/or ring, which may also be referred to as an anastomotic ring. In some embodiments, there is no distal implant segment 18 portion of implant 13 and the distal end of proximal implant segment 19 may comprise a continuous strut and/or ring with a skirt and/or flange that extends into the deep artery 4 and seals against the near wall of deep artery 4 upon deployment.

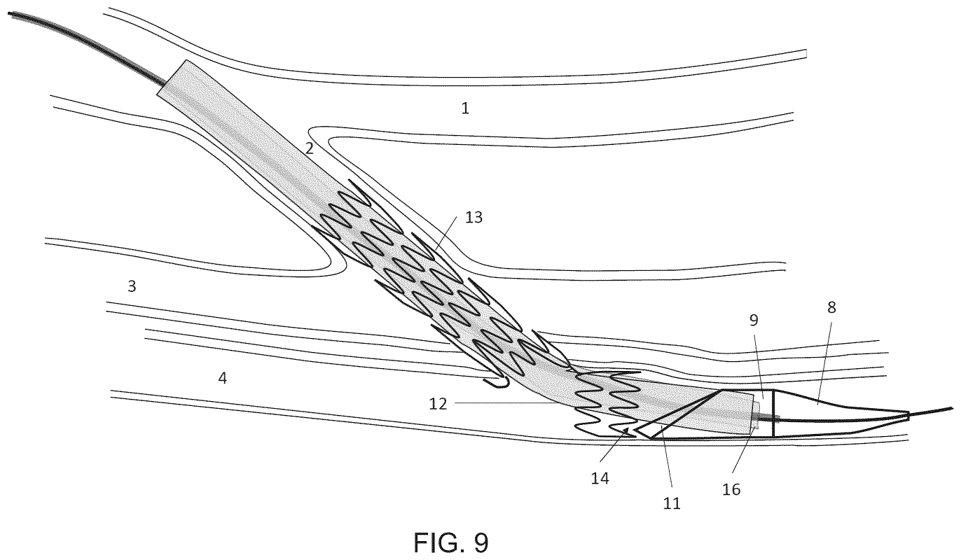

FIG. 9 depicts the initial step of removal of delivery system 29 with the advancement of outer sheath 12 and middle shaft 16 distally through the delivered (e.g., expanded) implant 13 and into cavity 9, according to some embodiments. In some embodiments, middle shaft 16 leads outer sheath 12 during this advancement step to facilitate reliable engagement of outer sheath 12 into cavity 9 without outer sheath 12 catching the proximal end 11 of distal nose cone 8.

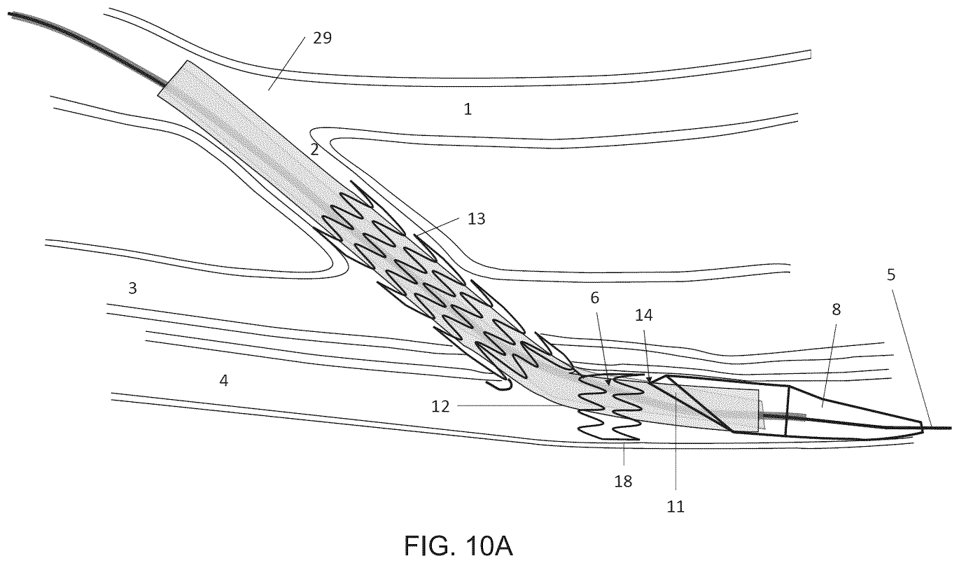

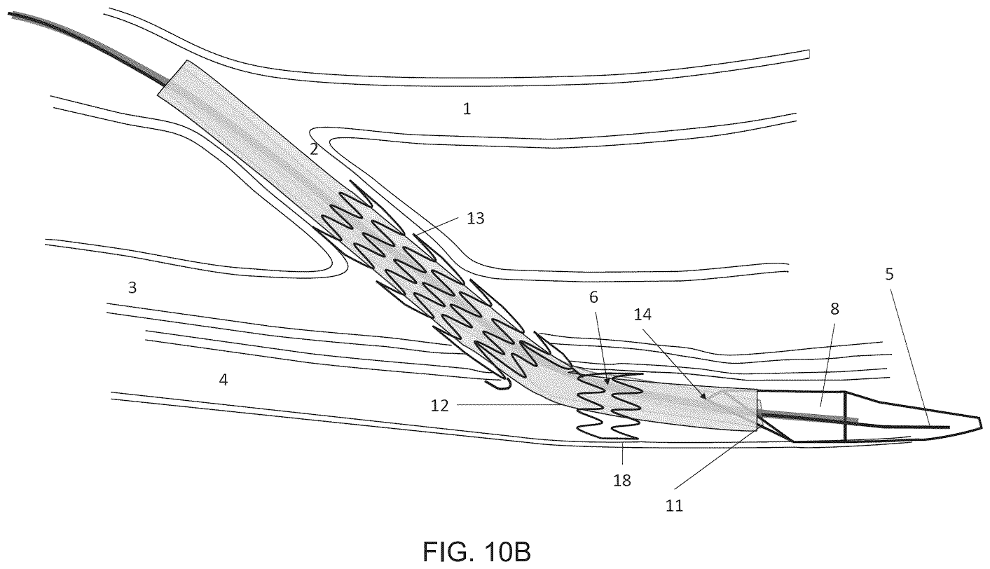

FIG. 10A depicts a continuation of the removal of delivery system 29 with the rotation of delivery system 29 around its axis of, e.g., approximately 180 degrees such that the proximal end (e.g., proximal-most end) of angled proximal end 11 of nose cone 8 is on the inside of curvature 6. In this orientation, gap 14 is minimized, eliminated, or substantially eliminated and there is flush contact between proximal tapered end 11 and outer sheath 12. This low-profile configuration facilitates removal of nose cone 8 without engagement of delivered implant 13 or any of the anatomical features near AVF location 7.

FIG. 10B depicts an alternative embodiment for providing a low-profile removal configuration for delivery system 29. In this embodiment, prior to advancing outer sheath 12 through implant 13, nose cone 8 is rotated, e.g., approximately 180 degrees around its axis. After rotation of nose cone 8, outer sheath 12 is advanced through implant 13 until it engages with the proximal end (e.g., proximal-most end) of angled proximal end 11. Due to the tapered structure of angled proximal end 11, it may enter the inner diameter of outer sheath 12 when outer sheath 12 is advanced. In this configuration, there are no structures on delivery system 29 that can interfere with its removal from the body. To complete removal in this embodiment, the delivery system is retracted while maintaining the overlap of outer sheath 12 over nose cone 8 until it exits the body.

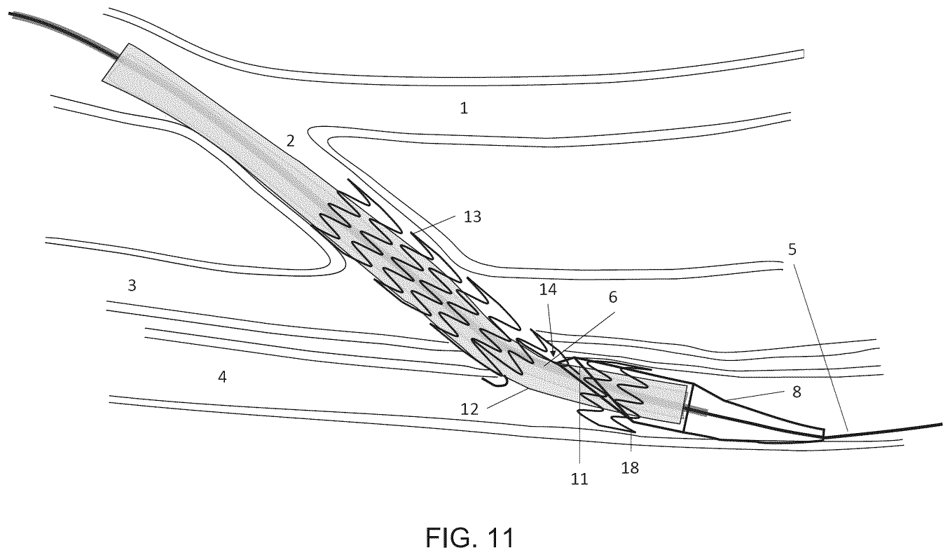

FIG. 11 depicts continuing of the removal of delivery system 29 in the configuration depicted in FIG. 10A. The angled proximal end has entered within delivered (e.g., expanded) distal implant segment 18 without interference due to the low-profile configuration. A preferred embodiment is where implant 13 has an unconstrained delivered (e.g., expanded) internal dimension which is greater than the outer dimension of nose cone 8 so that nose cone 8 does not experience excessive resistance or interference with implant 13 upon removal through implant 13.

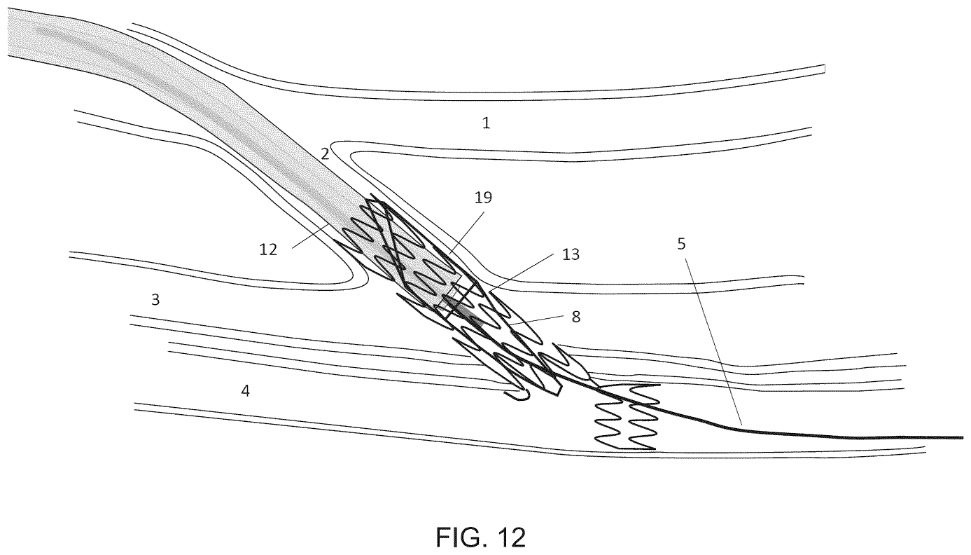

FIG. 12 depicts a further continuation of the removal of delivery system 29. Delivery system 29 has been further retracted and nose cone 8 has travelled partway through proximal implant segment 19 of implant 13.

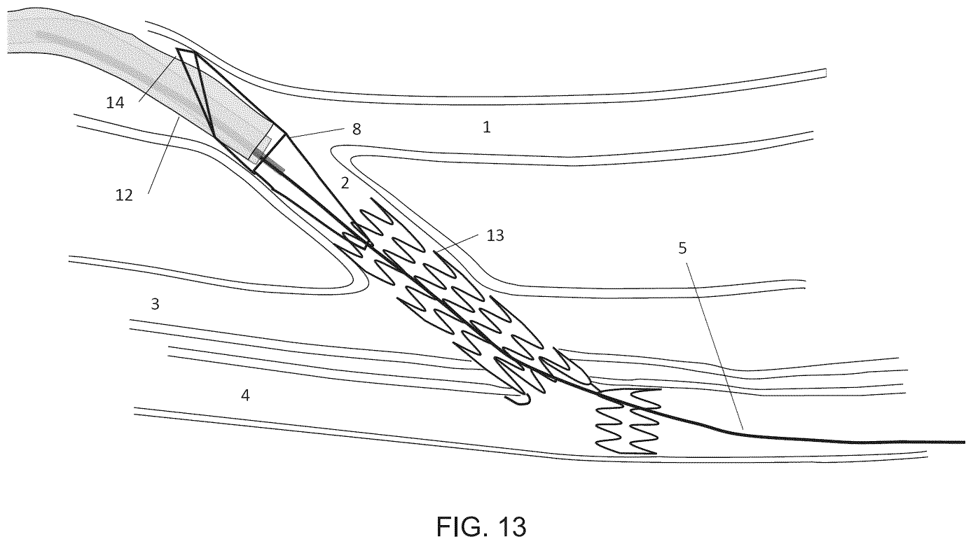

FIG. 13 depicts a continuation of the removal of delivery system 29. Delivery system 29 has been retracted completely through implant 13. Due to the curvature of the anatomy at this location, gap 14 may be formed again. If gap 14 causes undue resistance to the continuation of the removal of delivery system 29 from the body, delivery system 29 can be rotated once again to minimize and/or eliminate gap 14 and allow for minimal resistance to delivery system 29 removal.

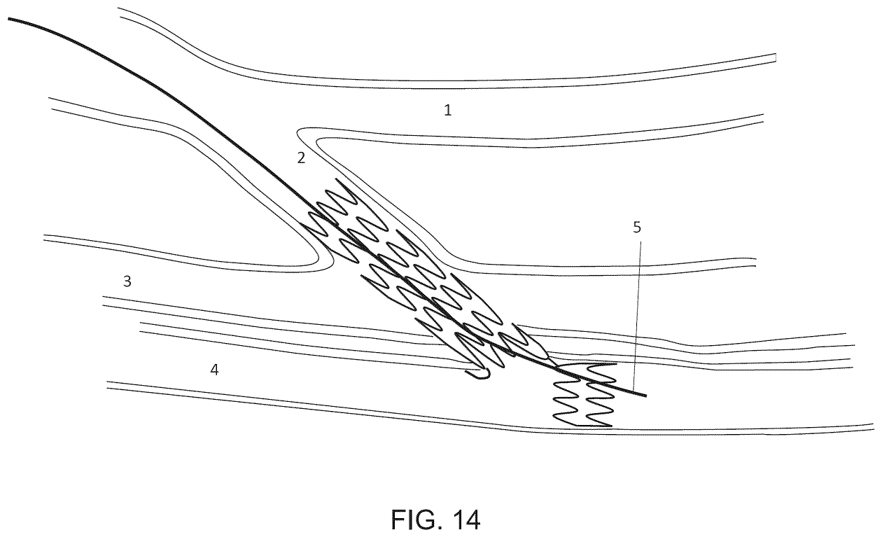

FIG. 14 depicts the initiation of removal of guide wire 5 from the body. Prior to removal of guidewire 5, it may be desirable or advantageous to advance a balloon dilatation catheter appropriately sized for implant 13 and the vasculature to facilitate complete expansion of implant 13. In some embodiments with a proximal implant segment 19 and a distal implant segment 18 of different diameters, cross-sectional areas, and/or perimeters, balloon dilation catheters of different sizes may be used to facilitate complete expansion of the proximal implant segment 19 and the distal implant segment 18. For example, in some embodiments, a 4 mm×20 mm or 2 mm×30 mm semi-compliant rapid exchange percutaneous transluminal angioplasty balloon can be used. A balloon dilation catheter can be used to facilitate complete expansion of the implant 13, such as when blood flow is insufficient (e.g., less than about 200 ml/min) to maintain complete expansion of the implant 13. One cause of insufficient blood flow through the implant 13 can be vascular spasm outside of the implant 13. When used, the balloon dilation catheter can extend throughout the implant 13 (both the distal implant segment 18 and proximal implant segment 19) and also target the adjacent vein and artery.

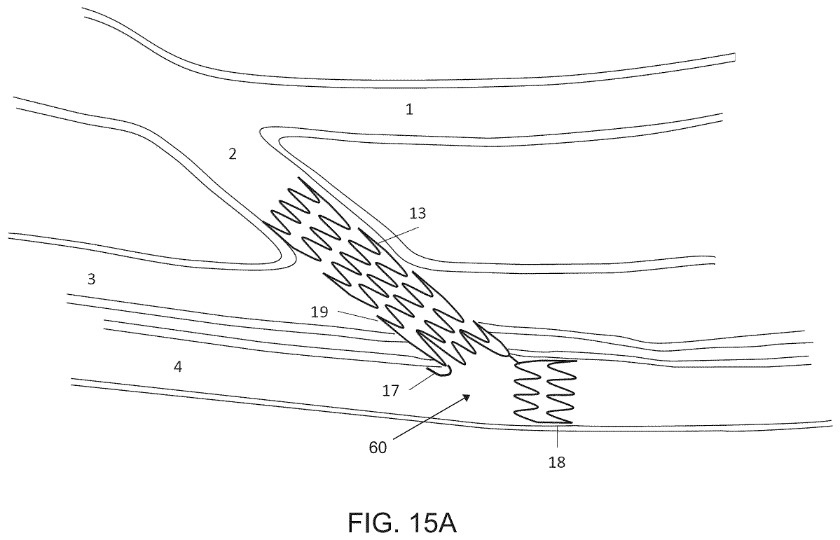

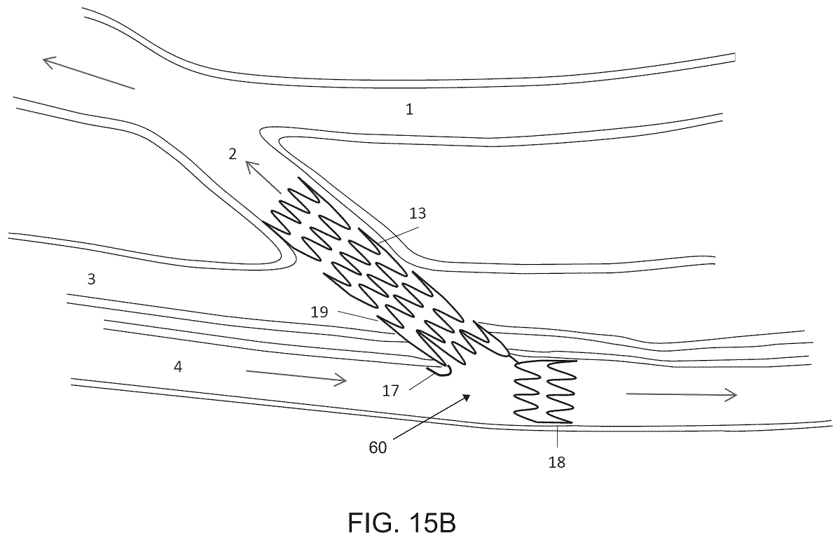

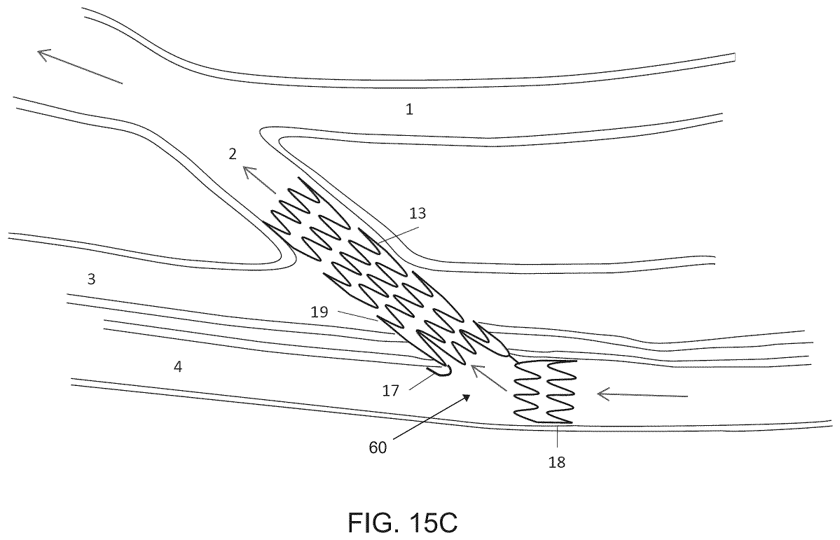

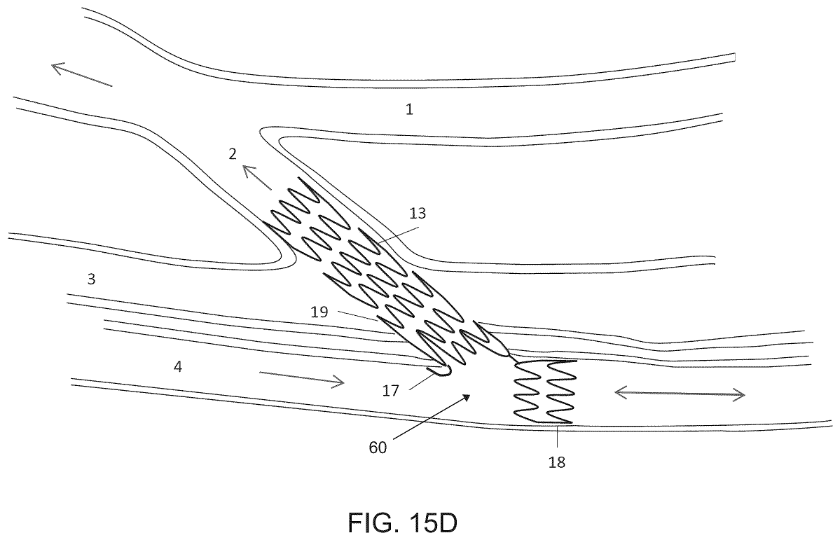

FIG. 15A depict the completed delivery of implant 13 with distal implant segment 18 in deep artery 4 and proximal segment 19 forming an AVF between at least one of deep vein 3, perforator vein 2, and superficial vein 1 with deep artery 4. In this embodiment implant 13 is at least partially anchored in place with anchor(s) 17 (which as shown face in the proximal direction) and distal implant segment 18. Alternative anchoring features such as barbs may also be used with some embodiments. Anchors and/or barbs of the implant 13 can extend in a proximal direction, a distal direction, bi-directionally (e.g., both proximal and distal), and/or at an angle relative to the proximal and distal directions. In some embodiments, the implant 13 can include one or more anchors that extend in a generally proximal direction at an angle between about 5 degrees to about 90 degrees, such as between about 10 degrees to about 40 degrees, relative to a surface of the implant 13 from which they extend. In some embodiments, the implant 13 can include one or more anchors that extend in a generally distal direction at an angle between about 5 degrees to about 90 degrees, such as between about 10 degrees to about 40 degrees, relative to a surface of the implant 13 from which they extend. Although the implant 13 is shown with the proximal implant segment 19 having anchors and/or barbs, in some embodiments the distal implant segment 18 can include anchors and/or barbs as described herein. In some embodiments, one or both of the proximal implant segment 19 and the distal implant segment 18 can comprise one or more anchors configured to anchor the one or both segments against a wall of a vein, a wall of an artery, and/or interstitial tissue.

In a preferred embodiment, implant 13 is covered or encapsulated with a biocompatible graft material such as, for example, ePTFE which can facilitate endovascular healing while minimizing stenosis of the lumen due to hyperplasia. In some embodiments, the graft material encapsulation can be constructed with a lamination of an inner layer of porous graft material, such as ePTFE, covering the inner surface of implant 13 and an outer layer of porous graft material, such as ePTFE, covering the outer surface of implant 13 that have been bonded together. In some embodiments, the bonding of the inner layer and outer layer of porous graft material encapsulates the struts of implant 13 and may be accomplished by fusing the outer and inner layers together with heat and compression. In these or other embodiments, a laminating layer of thermoplastic, such as fluorinated ethylene propylene (FEP) film, polyethylene (PE), or thermoplastic polyurethane film (TPU), may be placed between the inner and outer layer of porous graft material to facilitate the bonding. In some embodiments, the thermoplastic laminating layer may be porous and in other embodiments the laminating layer may be non-porous. In some embodiments, the porosity of the encapsulated implant 13 may be maintained by wrapping a strip of non-porous thermoplastic laminating layer in a helical fashion between the inner and outer layer of porous graft material and leaving gaps between each wrap of the thermoplastic laminating layer. In some embodiments, no gaps are left between each wrap of the thermoplastic laminating layer, leaving the final assembly non-porous, but with a porous surface. Covering or encapsulating proximal implant segment 19 with a graft material may prevent infiltration of blood into the or any interstitial tissues between deep vein 3 and deep artery 4 which may cause hematomas, infections and other complications. Covering proximal implant segment 19 with a graft material may also help divert blood flow from deep artery 4 into superficial vein 1.

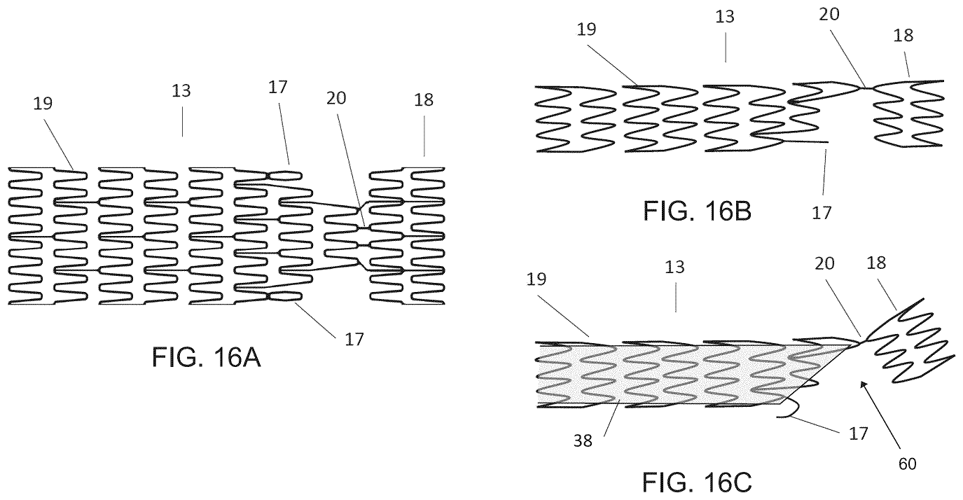

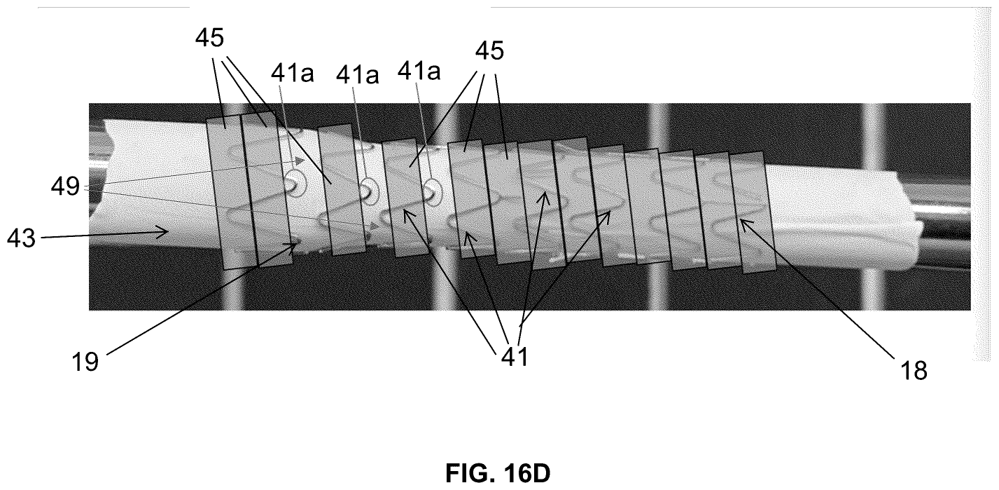

FIGS. 16A, 16B and 16C depict an embodiment of implant 13. FIG. 16A depicts a pattern that is intended to be cut from superelastic tubing, such as superelastic NiTi tubing, to form the features of implant 13 (e.g., the implant 13 can be formed from a single, laser cut hypotube). The sections of the cut pattern that form proximal implant segment 19, distal implant segment 18, anchor(s) 17 and connector struts 20 are shown. In some embodiments, implant 13 may alternatively be made of superelastic wire or formed from rolling cut superelastic sheet stock. FIG. 16B depicts the shape of implant 13 after the pattern in FIG. 16A has been cut out of a tube. FIG. 16C depicts implant 13 with distal implant segment 18, proximal implant segment 19 and connector struts 20 after it has been shape-set from superelastic tubing, such as superelastic NiTi tubing, using techniques that are well known. As shown, the proximal implant segment 19 and/or the distal implant segment 18 can comprise a plurality of struts.

Also depicted in FIG. 16C is a graft material 38 covering the inner diameter of proximal segment 19. Graft material 38 may be used to cover the inner diameter or outer diameter or both the inner and outer diameter of any portion (e.g., its entirety or less than its entirety) of implant 13 depending on the specific needs for the application. Expanded Polytetrafluoroethylene, also known as ePTFE has been shown to be an advantageous graft covering for endovascular implants. Other materials such as polyester mesh may also be suitable for certain embodiments. Implant 13 may be covered or encapsulated with a biocompatible graft material as described elsewhere herein.

In some embodiments, the proximal implant segment 19 may comprise an elongate tubular member or body with a proximal end, a distal end, an axis extending therethrough and a flow path/lumen therethrough. In some embodiments, the distal implant segment 18 may comprise an elongate tubular member or body with a proximal end, a distal end, an axis extending therethrough and a flow path/lumen therethrough. The proximal implant segment 19 and/or the distal implant segment 18 can be linearly straight when unconstrained (e.g., expanded) or as shape-set or in an at rest configuration as shown. Furthermore as shown, the proximal implant segment 19 can have an axial length greater than an axial length of the distal implant segment 18. In some embodiments, the distal implant segment 18 may be positioned downstream of the location of the distal end of the proximal implant segment 19 (e.g., downstream in regard to the direction of arterial blood flow). In some embodiments, implant 13 may not have proximal segments, distal segments, and/or anchoring features, and may have more basic structures that simply require accurate placement within the body.

In some embodiments, anchoring features of implant 13, such as anchor(s) 17, may form an angle relative to the body of the implant 13 of between about 10 degrees to about 90 degrees in the proximal and/or distal direction. In some embodiments, anchoring features of implant 13, such as anchor(s) 17, may form an angle relative to the body of the implant 13 of between about 35 degrees to about 40 degrees in the proximal and/or distal direction. Implant 13 may also be made from bioresorbable materials such as PLA, PGA, PLLA or other suitable materials for a specific application. Implant 13 can be coated on its internal surface, external/outer surface, or both internal and external/outer surface, with heparin and/or therapeutic agents, including drugs and compounds that are well known to reduce intimal hyperplasia and/or vascular stenosis in endovascular implant applications. In some embodiments, implant 13 can be coated on its internal surface, external/outer surface, or both internal and external/outer surface, with a hydrophilic coating. In some embodiments, implant 13 according to FIGS. 16A-16C may be only partially covered or encapsulated with a biocompatible graft material 38, include a laminating layer and/or a coating, for example, only proximal implant segment 19 may be covered/encapsulated with a biocompatible graft material, have a laminating layer, and be coated.

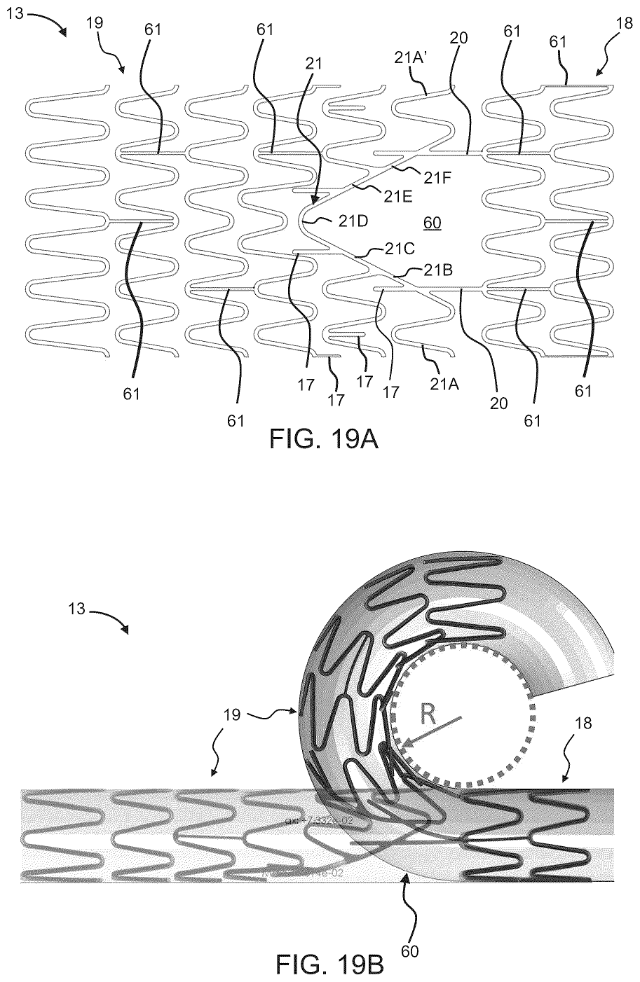

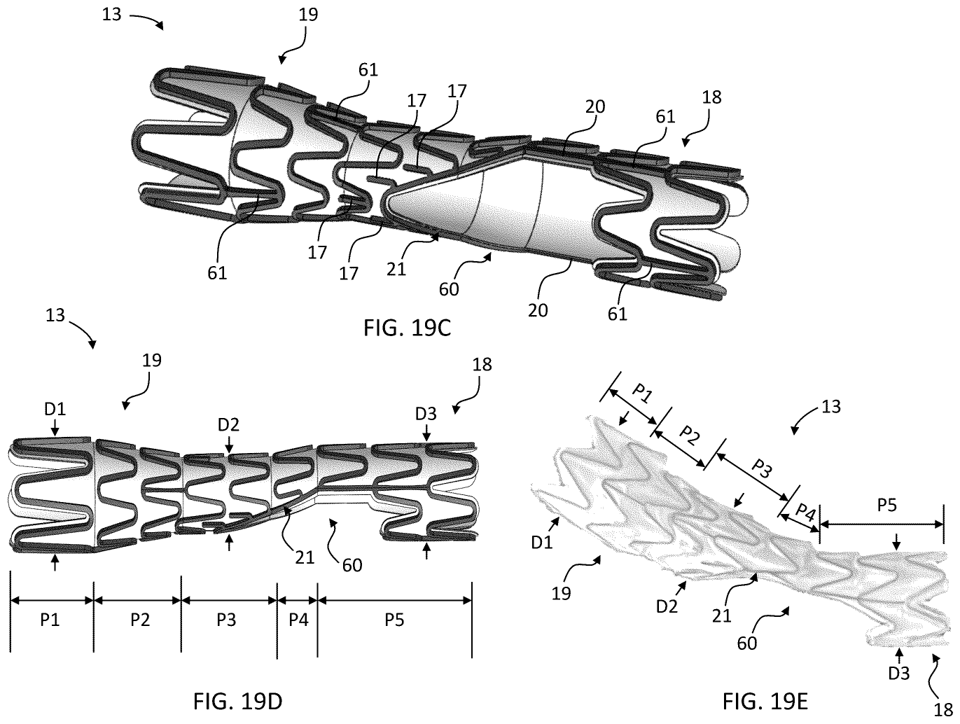

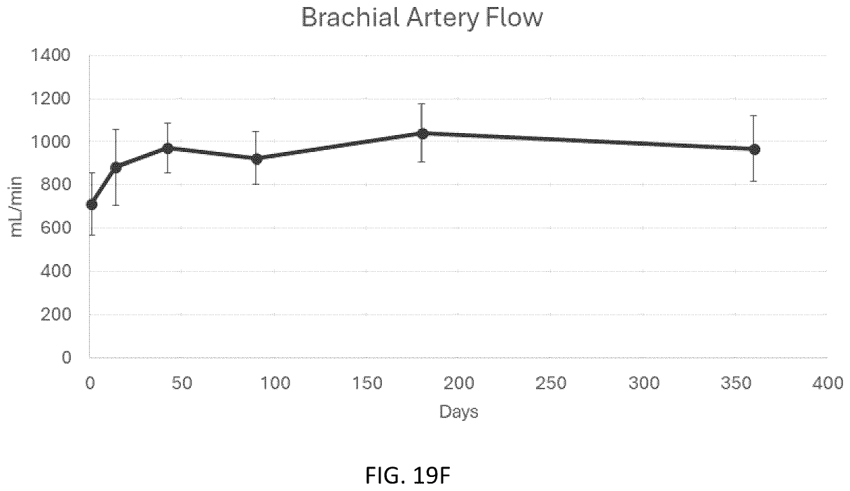

In some embodiments, as shown in FIG. 16D, during manufacture an implant frame 41 (e.g., the struts described herein) can be placed over an inner layer 43 of porous graft material, such as ePTFE. The inner layer 43 may extend the length of a segment of the implant 13 (e.g., the proximal implant segment 19) or the entire length of the implant 13. A helical wrapping of a strip 45 of thermoplastic laminating layer (e.g., FEP), schematically shown in FIG. 16D, which may be non-porous, can be applied around the implant frame 41 in such a way that no gaps are left on one side (e.g., a more distal segment) of the implant frame 41, but gaps 49 are left between each wrap or turn of the strip 45 on the other side (e.g., a more proximal segment) of the implant frame 41 to improve bending flexibility. Flexibility can be improved because the areas comprising wrapped strips 45 of thermoplastic laminating material are stiffer. Correspondingly, areas without the wrapped thermoplastic laminating material, such as exposed portions 41a of the implant frame 41 (e.g., the areas where there are gaps 49 in the helical wrapping of the strip 45 of thermoplastic laminating layer) can be more flexible. In some embodiments, the proximal implant segment 19 may comprise gaps 49 between one or more turns of the strip 45, e.g., between three adjacent turns. The strip 45 may be applied over some or all of the adjacent rows of struts in the proximal implant segment 19. In some embodiments, gaps 49 are formed in a proximal portion of the proximal implant segment 19, where additional flexibility may be desired, but not along a distal portion of the proximal implant segment 19, where a non-porous portion is desired to prevent infiltration of blood. In some embodiments, the gaps 49 between the turns of the strip 45 may be aligned with gaps between adjacent rows of struts of the implant frame 41. The exposed portions 41a of the implant frame 41 that correspond to gaps 49 that are not covered by the strip 45 and can have more flexibility. The flexibility of the proximal implant segment 19 can be increased, for example, to provide for the ability to bend such as shown in FIGS. 18D and 19B. A second or outer layer of porous graft material (not shown), such as ePTFE, may be applied over the wrapped strips 45 of the laminating layer, including over the exposed portions 41a of the implant frame 41. Pressure and heat may be used to melt the laminating layer and fuse the inner layer 43 and outer layers, with the implant frame 41 embedded in between.

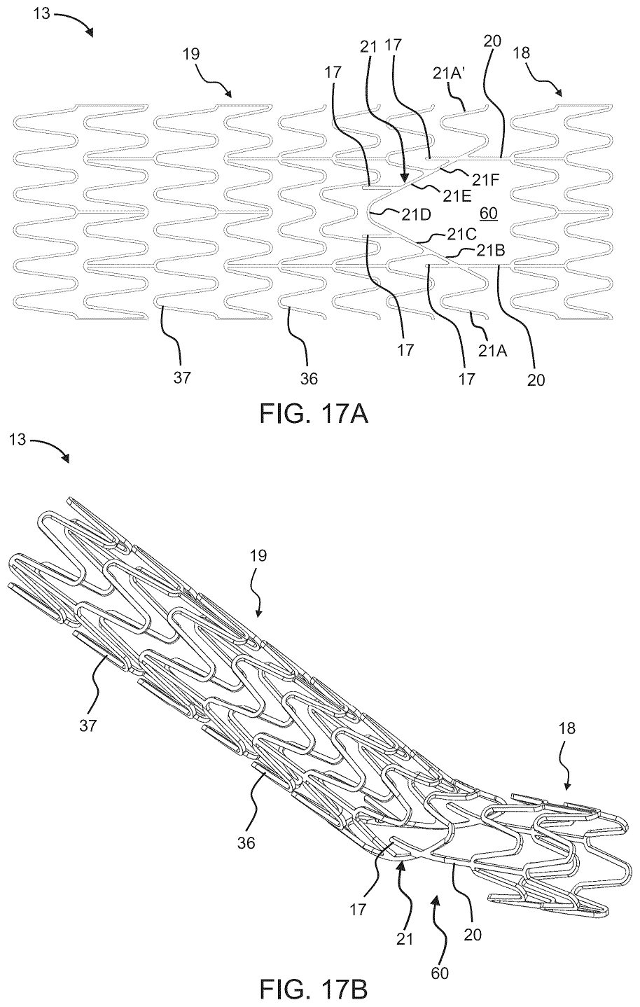

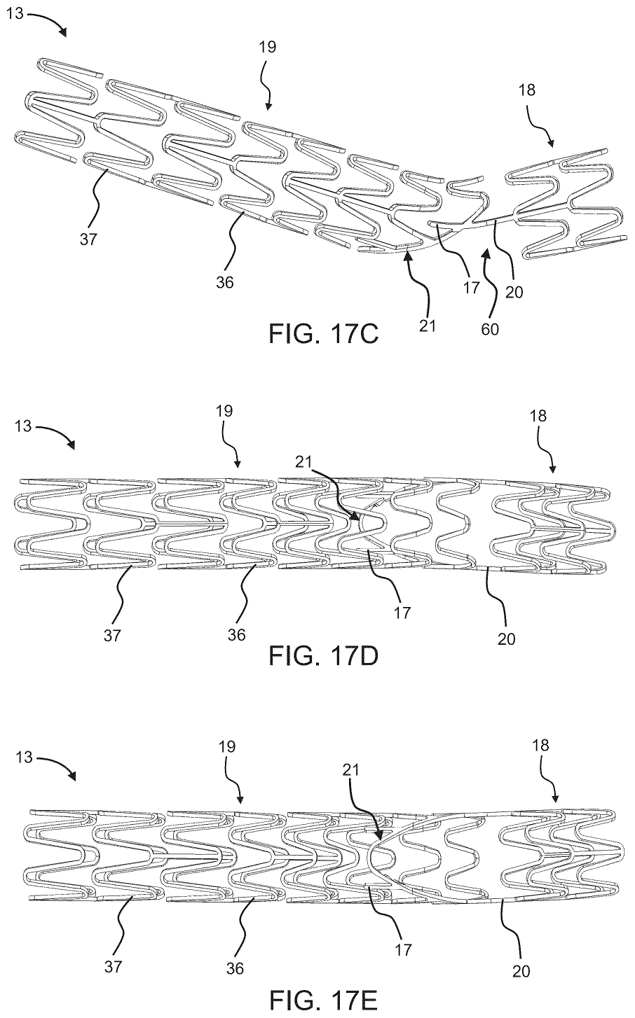

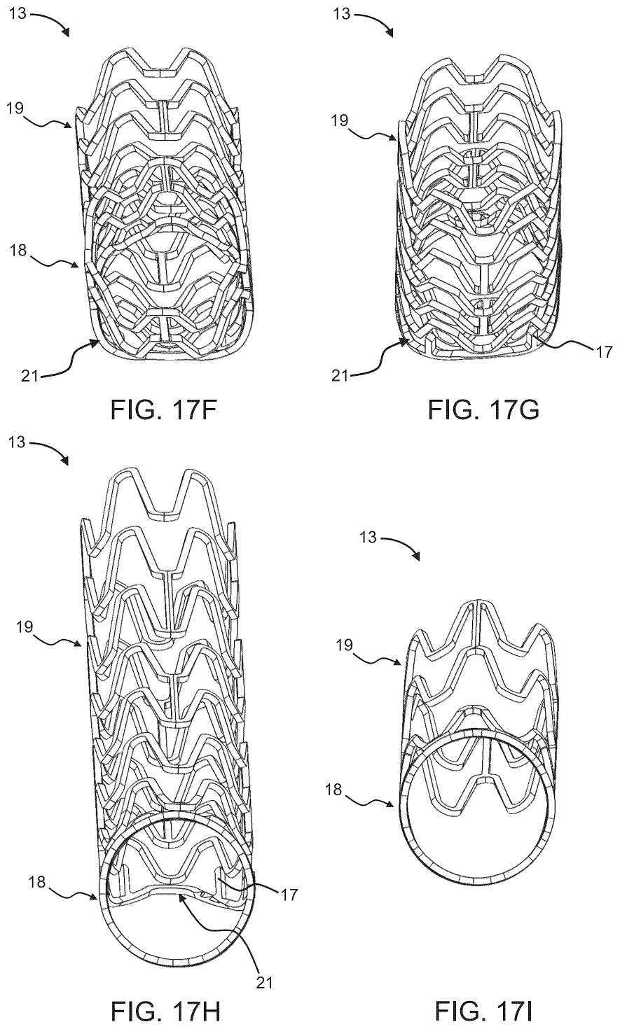

FIGS. 17A-17I depict an embodiment of implant 13. FIG. 17A depicts a pattern that is intended to be cut from superelastic tubing, such as superelastic NiTi tubing, to form the features of implant 13 (e.g., the implant 13 can be formed from a single, laser cut hypotube). The sections of the cut pattern that form proximal implant segment 19, distal implant segment 18, and connector struts 20 that connect the proximal implant segment 19 to the distal implant segment 18 are shown. In some embodiments implant 13 comprises anchor(s) 17 as additionally shown in the cut pattern. In some embodiments, the proximal implant segment 19 may comprise an elongate tubular member or body with a proximal end, a distal end, an axis extending therethrough and a flow path/lumen therethrough. In some embodiments, the distal implant segment 18 may comprise an elongate tubular member or body with a proximal end, a distal end, an axis extending therethrough and a flow path/lumen therethrough. In some embodiments, the distal implant segment 18 may be positioned downstream of the location of the distal end of the proximal implant segment 19 (e.g., downstream in regard to the direction of arterial blood flow).

In some embodiments implant 13 comprises a continuous strut/ring 21 (also referred to as an anastomotic ring) at the distal end of proximal implant segment 19 as additionally shown in the cut pattern (e.g., at the distal edge of the distal end of the proximal implant segment 19). As shown in FIG. 17A, continuous strut/ring 21 may comprise strut elements 21A, 21B, 21C, 21D, 21E, 21F, and 21A′, wherein strut elements 21A and 21A′ are continuous with one another (i.e., they are shown separate in FIG. 17A because it is a cut pattern; when cut into tubing, strut elements 21A and 21A′ are continuous with one another). In some embodiments, a continuous strut/ring 21 may provide a continuous distal edge to the proximal implant segment 19 to reduce wrinkles in an encapsulating graft material. In some embodiments, a continuous strut/ring 21 may provide a continuous distal edge to the proximal implant segment 19 to improve sealing of the implant 13 at the inner wall of deep artery 4. In some embodiments, a continuous strut/ring 21 may increase the radial stiffness of the distal edge of the proximal implant segment 19 to help maintain a fistula diameter and/or cross-sectional area (e.g., increased radial stiffness may lead to increased radial expansion of the fistula). In some embodiments, implant 13 comprises a continuous strut/ring 21 at the distal end of the proximal implant segment 19 and/or at the proximal end of the distal implant segment 18.

In some embodiments proximal implant segment 19 may comprise struts of different lengths, and additionally shown are the sections of the cut pattern that form struts 36 of a shorter length and struts 37 of a longer length. In some embodiments, in addition to being longer, struts 37 may have a greater thickness/width than struts 36. In some embodiments, the location of the longer and/or thicker/wider struts 37 may allow for a larger diameter and/or cross-sectional area of the implant 13 to be formed relative to the diameter and/or cross-sectional area of implant 13 where struts 36 are located. In some embodiments, the location of the longer and/or thicker/wider struts 37 may allow for an increased radial stiffness of the implant 13 relative to the radial stiffness of implant 13 where struts 36 are located. In some cases, thinner struts may used to reduce the radial stiffness of the implant where located. Sometimes, longer struts may decrease the radial stiffness of the implant where located. In some embodiments, the distal and/or proximal ends of the proximal and/or distal implant segments may comprise a reduced radial stiffness compared to a radial stiffness along their axial lengths (e.g., to reduce mechanical stress concentrations at the vessel/implant interface).

In some embodiments, implant 13 may alternatively be made of superelastic wire or formed from rolling cut superelastic sheet stock. FIG. 17B depicts a perspective view, FIG. 17C depicts a side view, FIG. 17D depicts a top view, and FIG. 17E depicts a bottom view of implant 13 with distal implant segment 18, proximal implant segment 19, connector struts 20, anchor(s) 17, continuous strut/ring 21, shorter struts 36, and longer struts 37 after it has been cut in superelastic tubing, such as NiTi tubing, from the pattern of FIG. 17A and shape-set. FIG. 17F depicts a front (distal end) view, FIG. 17G depicts a back (proximal end) view, FIG. 17H depicts a front (distal end) view through a longitudinal axis of distal implant segment 18, and FIG. 17I a back (proximal end) view through a longitudinal axis of proximal implant segment 19 of implant 13 with some of the various features identified in FIGS. 17A-17E after it has been cut in superelastic tubing, such as NiTi tubing, from the pattern of FIG. 17A and shape-set. Although not shown in FIGS. 17A-17I, the implant 13 of FIGS. 17A-17I may include any one or more of the features as described relative to an implant 13 described herein, including to implants of FIG. 8, FIGS. 15A-15C and FIGS. 16A-16C, such as being covered or encapsulated with a biocompatible graft material, having a laminating layer, and being coated with a hydrophilic coating and/or heparin and/or other therapeutic agents. In some embodiments, implant 13 according to FIGS. 17A-17I may be only partially covered or encapsulated with a biocompatible graft material, include a laminating layer and/or a coating, for example, only proximal implant segment 19 may be covered/encapsulated with a biocompatible graft material, have a laminating layer, and be coated.

FIGS. 18A-18C depict various embodiments of implant 13 after being shape-set and encapsulated with a biocompatible graft material as described herein. Implant 13 of FIG. 18A may correspond to implant 13 as described in FIGS. 17A-17I and is shown including distal implant segment 18, proximal implant segment 19, connector struts 20, anchor(s) 17, continuous strut/ring 21, shorter struts 36, and longer struts 37. As shown, and according to some embodiments, the section of proximal implant segment 19 where longer struts 37 are located has been formed to a diameter that is greater than a diameter of proximal implant segment 19 where shorter struts 36 are located. In other words, in some embodiments, the proximal implant segment 19 can be tapered such that the distal end of the proximal implant segment 19 has a different (e.g., smaller) diameter than the proximal end of the proximal implant segment 19. In alternative embodiments, such as shown in FIG. 18B, the proximal implant segment 19 of implant 13 may comprise struts of substantially similar length and have a substantially uniform diameter. FIG. 18B also shows various features of implant 13 as described herein, including distal implant segment 18, the aforementioned proximal implant segment 19, connector struts 20, anchor(s) 17, continuous strut/ring 21. Alternative embodiments of implant 13, such as shown in FIG. 18C, can include distal implant segment 18, proximal implant segment 19, connector struts 20, anchor(s) 17, continuous strut/ring 21, shorter struts 36, and longer struts 37. FIG. 18C also shows a proximal implant segment 19 that can be tapered such that the distal end of the proximal implant segment 19 has a different (e.g., smaller) diameter than the proximal end of the proximal implant segment 19. Moreover, the proximal implant segment 19 shown in FIG. 18C is shorter than those shown in FIGS. 18A and 18B, with 4 (as opposed to 6) struts 36 and 37. The embodiments shown in FIGS. 18A-18C emphasize that proximal implant segment 19 is understood to not be limited to a length shown in an individual figure. Similarly, the distal implant segment 18 is understood to not be limited to a length shown in an individual figure. Also as shown in FIGS. 18A-18C, the proximal implant segment 19 and/or distal implant segment 18 of implant 13 can be linearly straight when unconstrained or as shape-set or in an at rest configuration. Furthermore as shown, the proximal implant segment 19 can have an axial length greater than the distal implant segment 18. Additionally and as shown, the proximal implant segment 19 can be oriented at an angle relative to the distal implant segment 18 when the proximal and distal implant segments are fully expanded in an at rest configuration.

FIG. 18D depicts implant 13 according to FIGS. 17A-17I, FIGS. 18A-18C and as described herein in an unbent state and a bent state superimposed on one another. The unbent and bent states of implant 13 can correspond to the states of the implant 13 after implantation in a patient as described herein, such as when the patient's arm is substantially straight about the elbow (corresponding to the unbent state) and when the patient's arm is substantially bent at the elbow (corresponding to the bent state). As shown, the proximal implant segment 19 is configured to bend or curve relative to the distal implant segment in a direction away from the side opening 60. As shown, the proximal implant segment 19 can bend or curve relative to the distal implant segment 18 by at least about 90 degrees in a direction away from the side opening 60. In some embodiments, the proximal implant segment 19 is capable of bending or curving relative to the distal implant segment 18 by at least about 35 degrees, at least about 40 degrees, at least about 45 degrees, at least about 50 degrees, at least about 55 degrees, at least about 60 degrees, at least about 65 degrees, at least about 70 degrees, at least about 75 degrees, at least about 80 degrees, at least about 85 degrees, at least about 90 degrees, at least about 95 degrees, at least about 100 degrees, at least about 105 degrees, at least about 110 degrees, at least about 115 degrees, at least about 120 degrees, at least about 125 degrees, at least about 130 degrees, at least about 135 degrees, at least about 140 degrees, at least about 145 degrees, at least about 150 degrees, at least about 155 degrees, at least about 160 degrees, at least about 165 degrees, at least about 170 degrees, at least about 175 degrees, at least about 180 degrees, or more or any angle in a direction away from the side opening 60. The proximal implant segment 19 can bend or curve relative to the distal implant segment 18 and remain substantially patent/open so as to allow flow therethrough when bent or curved (e.g., blood flow through the proximal implant segment 19 is not reduced when it is bent). Furthermore, the proximal implant segment 19 can bend or curve relative to the distal implant segment 18 without kinking or ovalizing (e.g., becoming oval in cross-section). In other words, the proximal implant segment 19 can bend or curve relative to the distal implant segment 18 and remain substantially similar or the same in cross-sectional area as its unbent state. Such a configuration of the proximal implant segment 19 can advantageously minimize and or eliminate forces from the implant 13 on the vessels in which it is implanted when the vessel undergoes a bend and/or change in shape through movement of the patient (e.g., movement of the patient's arm about their elbow). The proximal implant segment 19 can bend or curve relative to the distal implant segment 18 with a radius of curvature R of the bend/curve of about 6 mm, about 5.5 mm, about 5.0 mm, about 4.5 mm, about 4.0 mm, about 3.5 mm, about 3.0 mm, about 2.9 mm, about 2.8 mm, about 2.7 mm, about 2.6 mm, about 2.5 mm, about 2.4 mm, about 2.3 mm, about 2.2 mm, about 2.1 mm, about 2.0 mm, less than about 2.0 mm or more than about 6 mm or any range therebetween.

FIG. 19A depicts another embodiment of an implant 13, specifically a pattern that is intended to be cut from superelastic tubing, such as superelastic NiTi tubing, to form the features of implant 13 (e.g., the implant 13 can be formed from a single, laser cut hypotube). The implant 13 according to FIG. 19A can be the same or similar to and/or incorporate any one or more of the features described and/or illustrated with respect to an implant 13 as described herein. For example, the implant 13 shown in FIG. 19A can include a proximal implant segment 19 comprising a plurality of struts with a continuous strut/ring 21 (e.g., an anastomotic ring) at its distal end, a distal implant segment 18 comprising a plurality of struts connected to the proximal implant 19 by connecting struts 20, a side opening 60 between the proximal and distal implant segments, and one or more anchors 17 facing in a proximal and/or distal direction. The implant 13 can be configured such that the proximal implant segment 19 has a different flexibility than the distal implant segment 18. For example and as will be discussed, the proximal implant segment 19 can be configured to have a greater flexibility than the distal implant segment 18. Increased flexibility of the implant 13, in particular of the proximal implant segment 19, can advantageously minimize stresses on the vessel(s) in which the implant 13 is implanted and allow the vessel(s) to move as they would normally without an implant 13. Stress on vessel(s) can promote intimal hyperplasia and sub-optimal healing, thus an implant 13 with increased flexibility can help avoid such conditions and promote healing.

The proximal implant segment 19 can comprise a series of rows of struts arranged substantially circumferentially about the axis of the proximal implant segment 19, which in some embodiments can comprise wavy struts as shown. These series of rows of struts of the proximal implant segment 19 can be connected to one another by one or more axially extending struts 61 arranged substantially along the axis of the proximal implant segment 19. Similarly, the distal implant segment 18 can comprise a series of rows of struts arranged substantially circumferentially about the axis of the distal implant segment 18, which in some embodiments can comprise wavy struts as shown. These series of rows of struts of the distal implant segment 18 can be connected to one another by one or more axially extending struts 61 arranged substantially along the axis of the distal implant segment 18. To achieve the aforementioned greater flexibility of the proximal implant segment 19 relative to the distal implant segment 18, in some embodiments the series of rows of struts of the distal implant segment 18 can be connected to one another by at least one more axially extending strut 61 than the series of rows of struts of the proximal implant segment 19. For example and as shown, adjacent rows of struts of the proximal implant segment 19 can be connected by no more than one axially extending strut 61, whereas adjacent rows of struts of the distal implant segment 18 can be connected by two (e.g., at least one more) axially extending struts 61.

In addition or alternatively to the number of axially extending struts of the proximal implant segment 19 having an effect on the relative flexibility thereof, the location of such axially extending struts of the proximal implant segment 19 can affect the flexibility of the proximal implant segment 19 (and likewise the flexibility of the distal implant segment 18). As shown in FIG. 19A, a majority or all of the one or more axially extending struts 61 of the proximal implant segment 19 can be located on a side of the proximal implant segment 19 aligned with a location of the side opening 60 (which, in the cut pattern shown corresponds with the middle portion of the implant 13 extending left to right). A side aligned with the side opening 60 can be up to and include about 90 degrees circumferentially on each side of a circumferential center of the side opening. As shown in FIG. 19A, the side aligned with the side opening 60 up to and including about 90 degrees circumferentially on each side of the side opening's circumferential center can include the approximate 180 degrees between the connector struts 20. By avoiding having axially extending struts 61 on a side of the implant 13 opposite the side opening 60, when the proximal implant segment 19 bends or curves, the adjacent rows of struts of the proximal implant segment can be brought closer to one another without an axially extending strut 61 preventing such movement. Furthermore, any graft material 38 of the proximal implant segment 19 in such area opposite the side opening can fold and/or wrinkle when the proximal implant segment 19 is bent or curved. In some implementations, the proximal implant segment 19 does not have axially extending struts 61 on a side opposite the side opening 60. In some implementations, the proximal implant segment 19 does not have axially extending struts 61 circumferentially beyond the side opening 60 and/or circumferentially beyond connecting struts 20. In some implementations, one or more axially extending struts 61 are circumferentially aligned with connecting struts 20.

In some embodiments, adjacent rows of struts arranged substantially circumferentially about the axis of the proximal implant segment 19 and/or the distal implant segment 18 can be connected by the graft material/covering 38 alone without any axially extending struts 61. In such embodiments, the implant frame or portions thereof can generally comprise an assembly of rows of struts held together by the graft material/covering 38 instead of being laser cut from tubing. Such an embodiment can provide for a proximal implant segment 19 and/or distal implant segment 18 with increased flexibility. In some cases and for any of the implants 13 described herein, the struts arranged substantially circumferentially about the axis of the proximal implant segment 19 and/or the distal implant segment 18 can be formed as rings instead of wavy struts, and/or can include a mix of rings and wavy struts.

FIG. 19B depicts implant 13 according to FIG. 19A and as described herein in an unbent state and a bent state superimposed on one another. The unbent and bent states of implant 13 can correspond to the states of the implant 13 after implantation in a patient as described herein, such as when the patient's arm is substantially straight about the elbow (corresponding to the unbent state) and when the patient's arm is substantially bent at the elbow (corresponding to the bent state). As shown, the proximal implant segment 19 is configured to bend or curve relative to the distal implant segment in a direction away from the side opening 60. As shown, the proximal implant segment 19 can bend or curve relative to the distal implant segment 18 by at least about 180 degrees in a direction away from the side opening 60. In some embodiments, the proximal implant segment 19 is capable of bending or curving relative to the distal implant segment 18 by at least about 35 degrees, at least about 40 degrees, at least about 45 degrees, at least about 50 degrees, at least about 55 degrees, at least about 60 degrees, at least about 65 degrees, at least about 70 degrees, at least about 75 degrees, at least about 80 degrees, at least about 85 degrees, at least about 90 degrees, at least about 95 degrees, at least about 100 degrees, at least about 105 degrees, at least about 110 degrees, at least about 115 degrees, at least about 120 degrees, at least about 125 degrees, at least about 130 degrees, at least about 135 degrees, at least about 140 degrees, at least about 145 degrees, at least about 150 degrees, at least about 155 degrees, at least about 160 degrees, at least about 165 degrees, at least about 170 degrees, at least about 175 degrees, at least about 180 degrees, at least about 185 degrees, at least about 190 degrees, at least about 195 degrees, at least about 200 degrees, at least about 205 degrees, at least about 210 degrees, at least about 215 degrees, or more or any angle in a direction away from the side opening 60. The proximal implant segment 19 can bend or curve relative to the distal implant segment 18 and remain substantially patent/open so as to allow flow therethrough when bent or curved (e.g., blood flow through the proximal implant segment 19 is not reduced when it is bent). Furthermore, the proximal implant segment 19 can bend or curve relative to the distal implant segment 18 without kinking or ovalizing (e.g., becoming oval in cross-section). In other words, the proximal implant segment 19 can bend or curve relative to the distal implant segment 18 and remain substantially similar or the same in cross-sectional area as its unbent state. Such a configuration of the proximal implant segment 19 can advantageously minimize and or eliminate forces from the implant 13 on the vessels in which it is implanted when the vessel undergoes a bend and/or change in shape through movement of the patient (e.g., movement of the patient's arm about their elbow). The proximal implant segment 19 can bend or curve relative to the distal implant segment 18 with a radius of curvature R of the bend/curve of about 6 mm, about 5.5 mm, about 5.0 mm, about 4.5 mm, about 4.0 mm, about 3.5 mm, about 3.0 mm, about 2.9 mm, about 2.8 mm, about 2.7 mm, about 2.6 mm, about 2.5 mm, about 2.4 mm, about 2.3 mm, about 2.2 mm, about 2.1 mm, about 2.0 mm, less than about 2.0 mm or more than about 6 mm or any range therebetween. The proximal implant segment 19 of implant 13 of FIG. 19A can have an increased flexibility relative to the proximal implant segment 19 of implant 13 shown in FIG. 18A via the configuration (e.g., the number and location) of axially extending struts 61.

FIGS. 19C-19E depict another embodiment of an implant 13. FIG. 19C shows a bottom perspective view of the implant 13 in a linear configuration after being partially shape set, FIG. 19D shows a side view of the implant 13 in a linear configuration after being partially shape set, and FIG. 19E shows a side view of the implant 13 after being fully shape set. The implant 13 according to FIGS. 19C-19E can be the same or similar to and/or incorporate any one or more of the features described and/or illustrated with respect to an implant 13 as described herein. For example, the implant 13 can be cut from superelastic tubing, such as superelastic NiTi tubing. As another example, the implant 13 shown in FIGS. 19C-19E can include a proximal implant segment 19 comprising a plurality of struts, a distal implant segment 18 comprising a plurality of struts connected to the proximal implant 19 by connecting struts 20, and a side opening 60 between the proximal and distal implant segments as described herein. The implant 13 can include a continuous strut/ring 21 (e.g., an anastomotic ring) at the distal end of the proximal implant segment 19 as described herein. In some implementations, implant 13 can include a continuous strut/ring (e.g., an anastomotic ring) at the distal end of the proximal implant segment 19 and/or at the proximal end of the distal implant segment 18. The implant 13 can also include one or more anchors 17 as described herein. In some implementations, implant 13 includes two or less connecting struts 20 (e.g., no more than two connecting struts 20). The implant 13 can be shape set into a form as described herein and shown in the example of FIG. 19E with the proximal implant segment 19 at an angle with respect to the distal implant segment 18. In another example, the implant 13 can comprise one or more layers of a graft material, a liner material, and/or a laminating material as described herein.

The implant 13 according to FIGS. 19C-19E can have an hourglass shape. The implant 13 can be shape set into such hourglass shape. In some implementations, the implant 13 can have an asymmetric hourglass shape. As shown in FIGS. 19D-19E, the implant 13 can have a first longitudinal portion P1, a second longitudinal portion P2, a third longitudinal portion P3, a fourth longitudinal portion P4, and a fifth longitudinal portion P5. The first longitudinal portion P1 can have a first diameter D1, the third longitudinal portion P3 can have a second diameter D2, and the fifth longitudinal portion P5 can have a third diameter D3. The second longitudinal portion P2 can have a diameter that transitions along its longitudinal length from the first diameter D1 to the second diameter D2, and the fourth longitudinal portion P4 can have a diameter that transitions along its longitudinal length from the second diameter D2 to the third diameter D3. The proximal implant segment 19 can comprise, in order from its proximal end to its distal end, the first, second, third and fourth longitudinal portions P1-P4. In some implementations, the proximal implant segment 19 comprises, in order from its proximal end to its distal end, the first, second, third and fourth longitudinal portions P1-P4, and a portion of longitudinal portion P5 (e.g., a proximal portion of longitudinal portion P5). The distal implant segment 18 can comprise the fifth longitudinal portion P5. In some implementations, the distal implant segment 18 comprises a portion of longitudinal portion P5 (e.g., a distal portion of longitudinal portion P5).

The side opening 60 can extend from at least a portion of the third longitudinal portion P3 to at least a portion of the fifth longitudinal portion P5 as shown. Thus, side opening 60 can be positioned along at least a portion of the third longitudinal portion P3, the longitudinal portion P4, and at least a portion of the fifth longitudinal portion P5. The side opening 60 can extend from the proximal implant segment 19 to the distal implant segment 18. The side opening 60 can be positioned at the connection of the proximal implant segment 19 with the distal implant segment 18. The proximal implant segment 19 (for example, the distal end thereof) can form at least a portion of the side opening 60. The distal implant segment 18 (for example, the proximal end thereof) can form at least a portion of the side opening 60. In some embodiments, the side opening 60 does not have struts and/or graft material extending thereacross (e.g., no struts, graft material, or portions of the implant 13 block, interfere, or are in the flow path of side opening 60). Such configuration can advantageously allow for future interventions, such as catheter interventions, to go through the vessel(s) in which the implant 13 is positioned (through the side opening 60 and/or through the proximal and/or distal implant segments). Such configuration can also advantageously provide for improved hemodynamics.

The diameters of the longitudinal portions of implant 13 that include the side opening 60 can be virtual diameters. For example, longitudinal portions P3-P5 which may not form fully enclosed longitudinal portions of implant 13 due to side opening 60, can have virtual diameters D3-D5, respectively. The second diameter D2 can be smaller than the first diameter D1 and the third diameter D3 to form the hourglass shape of implant 13. In some implementations, the third diameter D3 is smaller than the first diameter D1. Such implementations can form an asymmetric hourglass shape of implant 13. Distal implant segment 18 can be shorter in length than proximal implant segment 19.

The diameter D1 (e.g., inner diameter or outer diameter) of the first longitudinal portion P1 can be between about 2 mm to about 12 mm. For example, the diameter D1 of the first longitudinal portion P1 can be about 2 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8 mm, about 8.5 mm, about 9.0 mm, about 9.5 mm, about 10.0 mm, about 10.5 mm, about 11.0 mm, about 11.5 mm, or about 12.0 mm. In some implementations, the diameter D1 of the first longitudinal portion P1 can be less than about 2.0 mm or more than about 12.0 mm or any range therebetween.

The diameter D2 (e.g., inner diameter or outer diameter) of the third longitudinal portion P3 can be between about 0.5 mm to about 10 mm. For example, the diameter D2 of the third longitudinal portion P3 can be about 0.5 mm, about 1.0 mm, about 1.5 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8 mm, about 8.5 mm, about 9.0 mm, about 9.5 mm, or about 10.0 mm. In some implementations, the diameter D2 of the third longitudinal portion P3 can be less than about 0.5 mm or more than about 10.0 mm or any range therebetween.

The diameter D3 (e.g., inner diameter or outer diameter) of the fifth longitudinal portion P5 can be between about 2 mm to about 12 mm. For example, the diameter D3 of the fifth longitudinal portion P5 can be about 2 mm, about 2.5 mm, about 3.0 mm, about 3.5 mm, about 3.75 mm, about 4.0 mm, about 4.25 mm, about 4.5 mm, about 5.0 mm, about 5.5 mm, about 6.0 mm, about 6.5 mm, about 7.0 mm, about 7.5 mm, about 8 mm, about 8.5 mm, about 9.0 mm, about 9.5 mm, about 10.0 mm, about 10.5 mm, about 11.0 mm, about 11.5 mm, or about 12.0 mm. In some implementations, the diameter D3 of the fifth longitudinal portion P5 can be less than about 2.0 mm or more than about 12.0 mm or any range therebetween.

In an exemplary embodiment of the implant 13 according to FIGS. 19C-19E, the diameter D1 of the first longitudinal portion P1 can be about 6 mm, the diameter D2 of the third longitudinal portion P3 can be about 4 mm, and the diameter D3 of the fifth longitudinal portion P5 can be about 5 mm. In such exemplary embodiment, the diameters D1-D3 refer to inner diameters. Furthermore, in such exemplary embodiment the second longitudinal portion P2 can have a diameter that transitions along its longitudinal length from the diameter D1 of about 6 mm to the diameter D2 of about 4 mm, and the fourth longitudinal portion P4 can have a diameter that transitions along its longitudinal length from the diameter D2 of about 4 mm to the diameter D3 of about 6 mm. Thus, in this exemplary embodiment, the implant 13 comprises an asymmetric hourglass shape.

An implant 13 with an hourglass shape as shown in FIGS. 19C-19E can advantageously reduce a risk of a pseudoaneurysm or mechanical stress on the vessel in patients receiving the implant 13. For instance, the diameter D1 of the first longitudinal portion P1 and the diameter D3 of the fifth longitudinal portion P5 can each be oversized relative to the diameters of the vessels in which the proximal implant segment 19 and the distal implant segment 18, respectively, will be implanted. Such oversizing can advantageously prevent micromotion between the implant 13 and the vessels of the patient when the patient moves (e.g., flexes their arm about their elbow in an implant 13 implanted within the arm of the patient as described herein). For instance, diameter D2 of the third longitudinal portion P3 can be smaller than the diameters D1 and D3, creating the hourglass shape in implant 13 and leading to the third longitudinal portion P3 functioning as a flow restrictor (which can also be referred to herein as a “flow regulator”) of the implant 13. In other words, the implant 13 can have oversized portions at its proximal and distal ends and a flow restrictor in between (e.g., portions at the proximal and distal ends of the implant 13 can have a greater cross-sectional area than the flow restrictor portion). In some implementations and as shown in FIGS. 19C-19E, such a flow restrictor portion (e.g., longitudinal portion P3 with diameter D2) can be positioned at or adjacent the side opening 60. The implant 13, by having a flow restrictor/regulator, can advantageously control flow dynamics through the fistula created thereby.

In some embodiments, the diameter D1 of the first longitudinal portion P1 can be oversized relative to a vessel in which the proximal implant segment 19 will be implanted by between about 10% to about 80%, such as about 10%, about 15%, about 20%, about 25%, about 30%, about 35%, about 40%, about 45%, about 50%, about 55%, about 60%, about 65%, about 70%, about 75%, or about 80%. In some embodiments, the diameter D3 of the fifth longitudinal portion P5 can be oversized relative to a vessel in which the distal implant segment 18 will be implanted by between about 10% to about 100%, such as about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%.

The implant 13 can advantageously ensure that a minimum of fistula flow is created by the implant 13 after implantation. Such minimum fistula flow can be defined as flow that goes through the proximal implant segment 19 from an artery to a vein as described herein, and can be greater than about 300 ml/min, greater than about 400 ml/min, greater than about 500 ml/min, greater than about 600 ml/min, greater than about 700 ml/min, greater than about 800 ml/min, greater than about 900 ml/min, greater than about 1000 ml/min, greater than about 1100 ml/min, greater than about 1200 ml/min, greater than about 1300 ml/min, or greater than about 1400 ml/min. The hourglass shape of implant 13 can also advantageously ensure that excess fistula flow is not created by the implant 13 after implantation. Such maximum fistula flow can be defined as flow that goes through the proximal implant segment 19 from an artery to a vein as described herein, and can be less than about 2000 ml/min, less than about 1900 ml/min, less than about 1800 ml/min, less than about 1700 ml/min, less than about 1600 ml/min, less than about 1500 ml/min, less than about 1400 ml/min, less than about 1300 ml/min, less than about 1200 ml/min, less than about 1100 ml/min, less than about 1000 ml/min, less than about 900 ml/min, less than about 800 ml/min, less than about 700 ml/min, less than about 600 ml/min, or less than about 500 ml/min. In some implementations, the implant 13 in configured to produce a flow of blood through a fistula created thereby in a range of about 300 ml/min to about 1800 ml/min, of about 400 ml/min to about 1700 ml/min, of about 500 ml/min to about 1600 ml/min, of about 600 ml/min to about 1500 ml/min, of about 700 ml/min to about 1400 ml/min, of about 800 ml/min to about 1300 ml/min, of about 900 ml/min to about 1200 ml/min, of about 900 ml/min to about 1100 ml/min, of about 1000 ml/min, or of about 1100 ml/min. Such minimum and/or maximum fistula flow can be controlled by the smallest diameter portion of the implant 13, which in an hourglass implant can be the diameter D2 of longitudinal portion P3. In some implementations, the implant 13 is configured to produce a flow of blood through a fistula created thereby in a range of about 1000 ml/min to about 1400 ml/min, which can be advantageous for producing a reliable access point (e.g., for producing a mature superficial vein) for dialysis.

FIG. 19F shows a plot of clinical results after implantation of an implant 13 as described herein. Such clinical results were gathered after implantation of an hourglass shaped implant 13 according to FIGS. 19C-19E. Shown in the plot is blood flow through the brachial artery in ml/min versus days after implantation, with day 0 being the day of implantation. Flow in the brachial artery is a clinically used surrogate for flow through the fistula created by the implant 13. As can be seen, the implant 13 at day 0 produces a minimum flow rate through the fistula of about 700 ml/min. Upon maturation of the vasculature as a result of the implant 13 creating the fistula, blood flow through the fistula is shown to advantageously reach about 1000 ml/min within weeks of implantation, and maintained at about 1000 ml/min out to one year after implantation. In some embodiments, implantation of an implant as described herein may result in a brachial artery flow after 6 weeks of between about 600 ml/min to about 2000 ml min, more preferably about 800 ml/min to about 1500 ml/min.

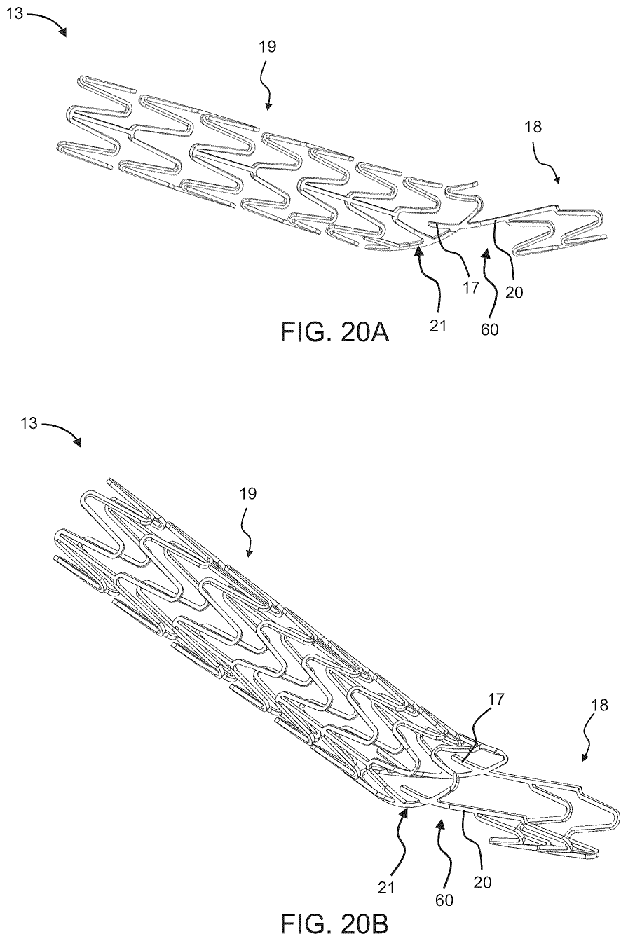

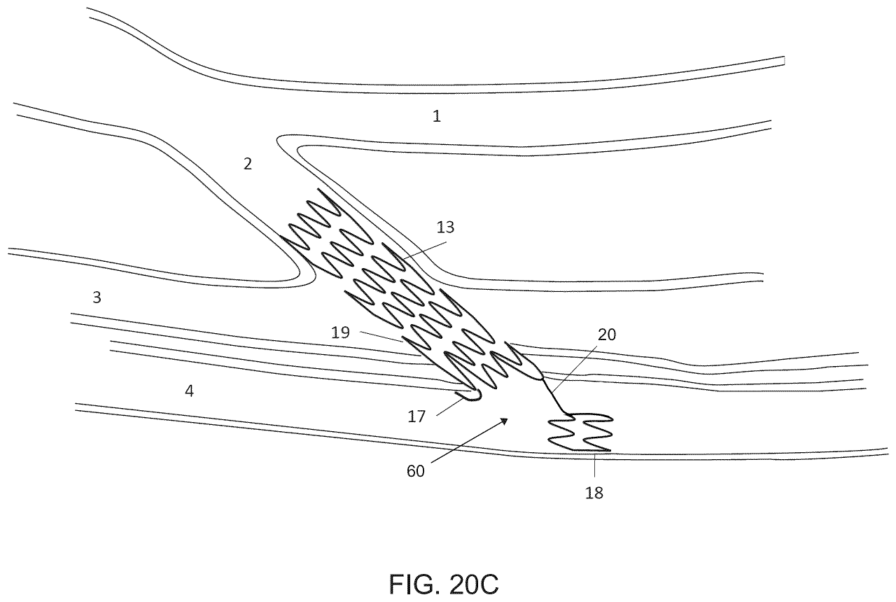

FIGS. 20A-20B depict another embodiment of an implant 13, specifically an implant cut from superelastic tubing, such as superelastic NiTi tubing, and shape set into form as described herein. FIG. 20A shows a side view and FIG. 20B shows a perspective view of implant 13. The implant 13 according to FIGS. 20A-20B can be the same or similar to and/or incorporate any one or more of the features described and/or illustrated with respect to an implant 13 as described herein. For example, the implant 13 shown in FIGS. 20A-20B can include a proximal implant segment 19 comprising a plurality of struts with a continuous strut/ring 21 (e.g., an anastomotic ring) at its distal end, a distal implant segment 18 comprising a plurality of struts connected to the proximal implant 19 by connecting struts 20, a side opening 60 between the proximal and distal implant segments, and one or more anchors 17. Different than other embodiments described herein, the distal implant segment 18 of implant 13 according to FIGS. 20A-20B can be configured as an at least partially tubular body extending between its proximal end to its distal end. In such a configuration, the distal implant segment 18 can provide at least partial radial support for the wall of the artery opposite from the distal end of the proximal implant segment, as shown in FIG. 20C wherein the implant 13 is implanted within the patient's vasculature. The distal implant segment 18 according to FIGS. 20A-20B is shown having a partial tubular body that extends about 180 degrees circumferentially from one connecting strut 20 to another connecting strut 20, although other configurations of the distal implant segment 18 can include a partial tubular body that extends less than about 180 degrees circumferentially (in which case the connecting struts 20 can be configured differently), or more than about 180 degrees circumferentially. For example, the distal implant segment 18 can be configured as a partially tubular body that extends about 30 degrees, about 45 degrees, about 60 degrees, about 75 degrees, about 90 degrees, about 105 degrees, about 120 degrees, about 135 degrees, about 150 degrees, about 165 degrees, about 180 degrees, about 195 degrees, about 205 degrees, about 220 degrees, about 235 degrees, about 250 degrees, about 275 degrees, about 290 degrees, about 305 degrees, about 320 degrees, about 335 degrees, or about any degrees above, between, or greater than those listed circumferentially. As shown, the partial tubular body forming the distal implant segment in one example may be oriented or aligned on the same side of the implant as the side opening 60.

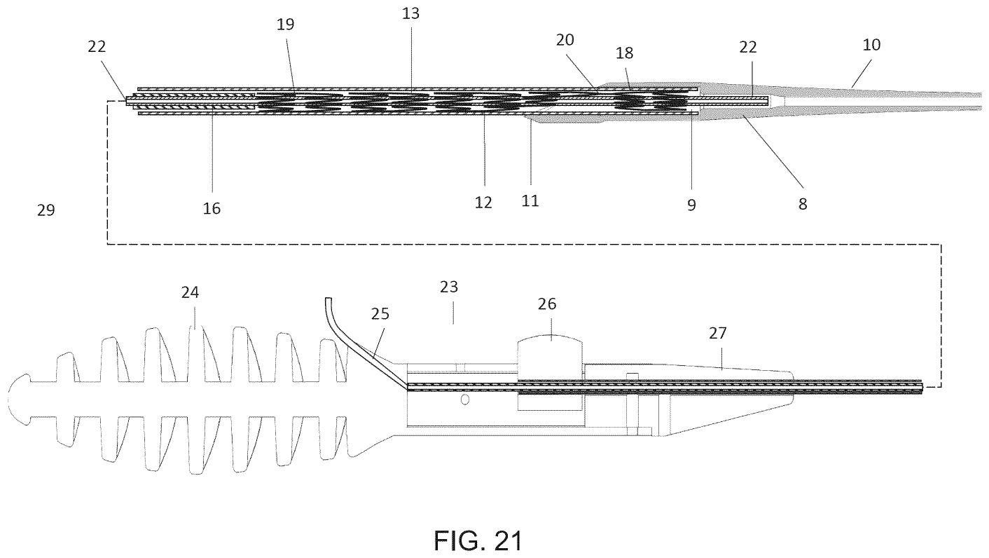

FIG. 21 depicts a partial cross-sectional view of the various elements of one embodiment of delivery system 29. Nose cone 8 with distal taper 10 and angled proximal end 11 is shown connected to guidewire shaft 22. Guidewire shaft 22 has an internal lumen which accommodates guidewire 5 (shown elsewhere). Middle shaft 16 is slidably disposed over guidewire shaft 22 and slidably disposed within outer sheath 12 and abuts the proximal end of constrained (e.g., collapsed) implant 13. The proximal end of middle shaft 16 terminates where it may be fixed to the proximal handle 23 or allowed to move relative to handle 23. Middle shaft 16 prevents implant 13 from moving relative to guidewire shaft 22 during retraction of outer sheath 12 during delivery of implant 13. Implant 13 is elastically constrained (e.g., collapsed) within outer sheath 12. Outer shaft 12 is attached to control knob 26 which is slidably attached to handle 23 so that a controlled retraction of outer sheath 12 can be achieved by sliding control knob 26 proximally. The distal end of outer sheath 12 terminates within cavity 9 of nose cone 8. At its proximal end, guidewire shaft 22 is fixed to handle 23 and is luminally connected to tube 25 which provides conduit for guidewire 5 to travel completely through delivery system 29. A hemostatic valve may be fitted on the proximal end of tube 25 to prevent bleed back when delivery system 29 is inserted in the vasculature. Also depicted in FIG. 21 are proximal handle 24 which facilitates manipulation of delivery system 29 and some of the constrained (e.g., collapsed) elements of implant 13 including distal implant segment 18, proximal implant segment 19 and connector struts 20. Handle nose cone 27 provides a guide path and support for outer sheath 12 which is slidably disposed within handle nose cone 27. FIG. 21 depicts a preferred embodiment of delivery system. Alternative preferred embodiments for delivery system 29 could include thumb wheel control of outer sheath 12 and pull wire features to allow active deflection of nose cone 8 to create gap 14 rather than relying on the curvature 6 of guidewire 5 as shown in FIG. 6. The materials of construction of delivery system 29 can be any of the materials well known to be used for catheter construction such as PEEK, HDPE, PeBax, nylon, PTFE, combinations thereof, and others. The diameter and length of delivery system 29 should be suited to the particular application. In one embodiment, the profile of nose cone 8 is between 6 Fr. and 9 Fr. for an implant 13 diameter of between 3 mm and 6 mm. In one embodiment, the distance between handle 23 and nose cone 8 is between about 15 cm and about 30 cm. Various lengths and diameters of delivery system 29 can be used in various embodiments of this invention to satisfy the needs of the particular application.

FIG. 22A depicts a 3-D view and a cross-sectional view of a nose cone 8. The nose cone 8 can comprise a generally tubular body with a distal end, a proximal end, and a longitudinal axis extending therebetween. According to some embodiments, angled proximal end 11 is shown with a profile intended to engage tissue when gap 14 is created and to not engage tissue or implant structures when gap 14 is eliminated. In some embodiments, angled proximal end 11 may engage tissue without a gap 14 (such as shown and described with respect to FIG. 22B). In some cases, angled proximal end 11 may engage tissue when it is oriented substantially on the outside of a curve (e.g., such as the curvature 6), and may otherwise not engage tissue when it is oriented substantially on the inside of a curve. The nose cone 8 can include a proximal opening forming a cavity (e.g., central lumen) 9 with dimensions sufficient to accept outer sheath 12 (not shown). The nose cone 8 can also include an opening extending longitudinally from a distal end of the cavity 9 through the tapered distal end 10 to allow for a guidewire (e.g., guidewire 5) to extend longitudinally through the nose cone 8. The proximal end of the nose cone 8 can in some embodiments be oblique to the longitudinal axis of the nose cone 8. As shown, a proximalmost tip 63 of the nose cone 8 can be radially aligned with an inner diameter of the cavity 9. Furthermore and as shown in the longitudinal cross-section at bottom, the proximal end 11 of the nose cone 8 can have a surface that extends distally and radially outward from the proximalmost tip 63 to an outer diameter of the nose cone, creating the angled proximal end 11. To improve the flexibility between nose cone 8 and outer sheath 12, a slit or slot may be included in the wall that forms cavity 9 (e.g., a longitudinal slit or slot), preferably in the region of the shorter wall segment forming cavity 9 shown in the cross sectional view of nose cone 8. Nose cone 8 includes a tapered distal section 10 which facilitates sliding over guide wire 5 (not shown) and passing through anatomical structures that have not yet been dilated to a diameter equal to or greater to the outer diameter of nose cone 8. The tapered section 10 can taper distally in some cases as illustrated. Nose cone 8 can be constructed from materials well known to be suitable for catheters such as, for example, PEEK, HDPE and Polypropylene. In some embodiments, nose cone 8 may include echogenic features to aid in ultrasound visualization.

FIG. 22B depicts a 3-D view and a cross-sectional view with a magnification of the angled proximal end 11 of a nose cone 8 according to some embodiments. The nose cone 8 according to FIG. 22B can be the same or similar to and/or incorporate any one or more of the features described and/or illustrated with respect to a nose cone 8 as described herein, such as the nose cone described with respect to FIG. 22A. Different than other embodiments described herein, the angled proximal end 11 of nose cone 8 according to FIG. 22B can be configured to engage tissue without requiring a gap 14 to be formed (e.g., without requiring a gap between an inner diameter of the cavity 9 and the outer sheath 12 when the outer sheath 12 is disposed within the cavity 9). To accomplish this, the proximal-most tip 63 of the nose cone 8 can be spaced radially outward of the inner diameter of the cavity 9 as shown in FIG. 22B. Said in another way, the proximal-most tip 63 can be spaced radially outward of a virtual projection of the inner diameter of the proximal cavity 9 to form a radial gap between the proximal-most tip 63 and the virtual projection of the inner diameter. This radial gap allows the proximal-most tip 63 to engage tissue such as the wall of the vein or the artery in which the nose cone is positioned. The angled proximal end 11 of the nose cone 8 according to FIG. 22B can thus, as shown in the longitudinal cross-section and its magnification, comprise a first surface 64 extending proximally and radially outwardly away from the inner diameter of the proximal cavity to the proximal-most tip 63, and a second surface 65 extending distally and radially outwardly from the proximal-most tip 63 to an outer diameter of the nose cone. The first surface 64 can engage the near wall of deep artery 4 after delivery system 29 has been pulled proximally from its location, as shown in FIG. 5, and does not require the formation of gap 14, shown in FIG. 5. The nose cone 8 as shown and described with respect to FIG. 22B can be incorporated in any of the delivery devices 29 described herein.

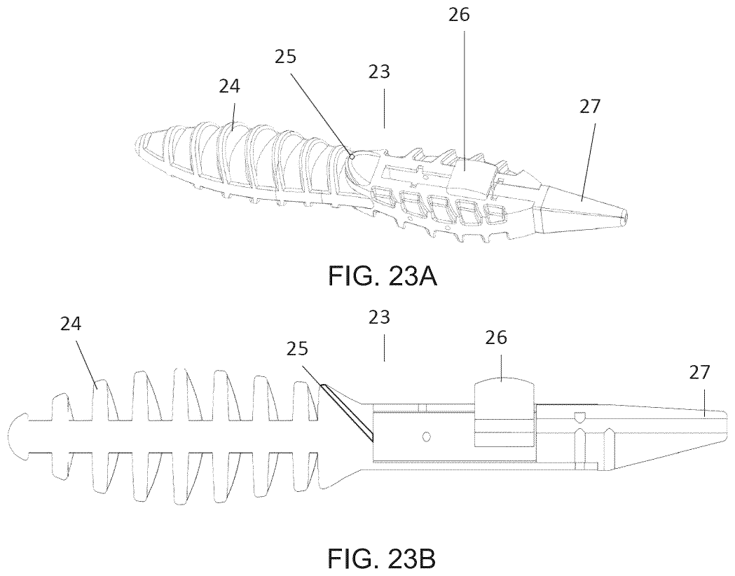

FIGS. 23A-23B depicts a 3-D view and cross-sectional view of handle 23 and its various elements. Handle nose cone 27 is shown attached to the distal end of handle 23 with a thru-lumen that slidably accommodates outer sheath 12 (not shown). Control knob 26 is slidably constrained within handle 23 and is used to control the axial position of outer sheath 12 (not shown). Tube 25 allows for passage of guidewire 5 (not shown) through handle 23.

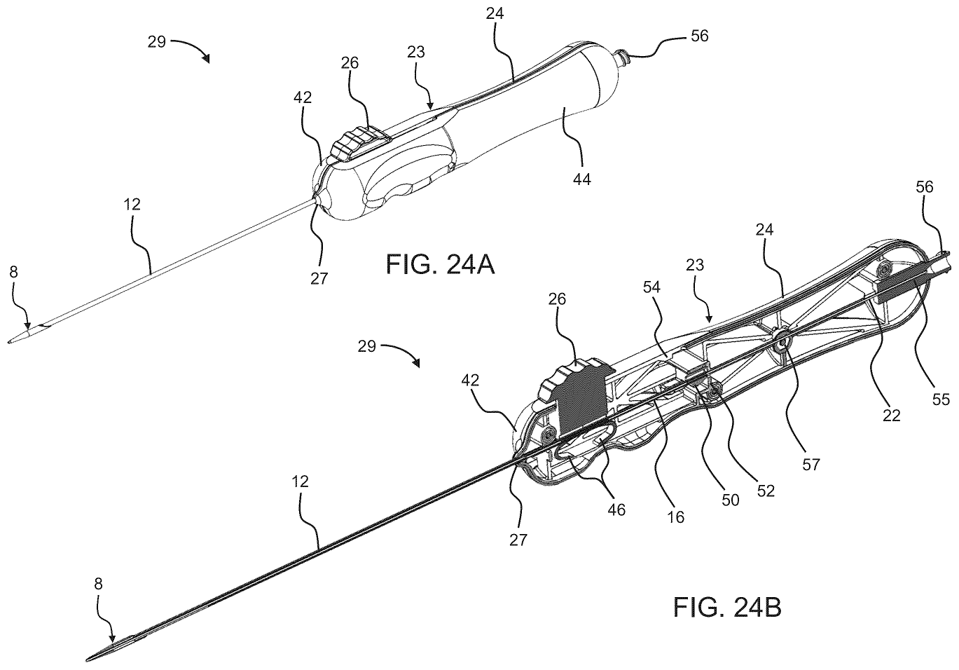

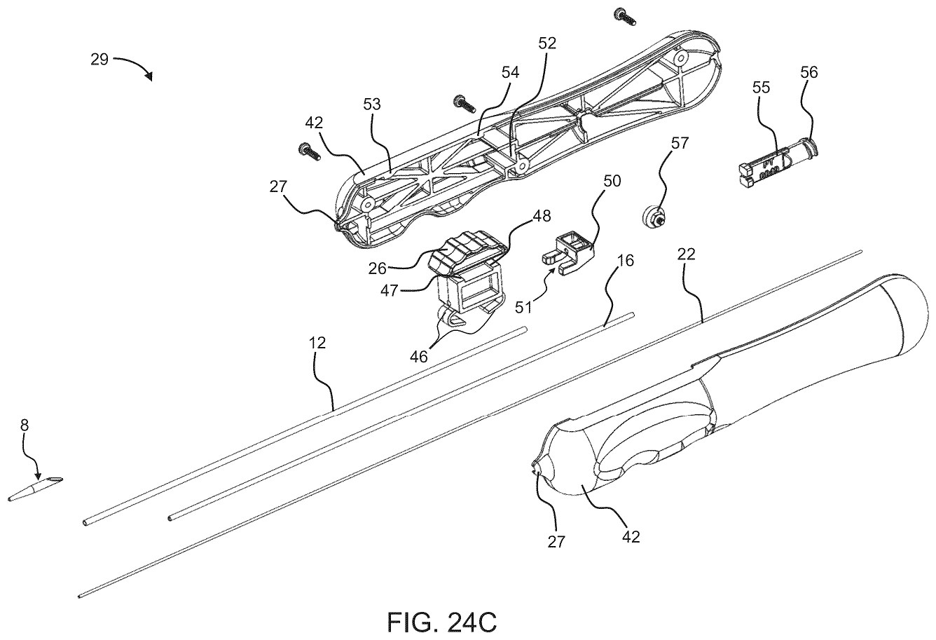

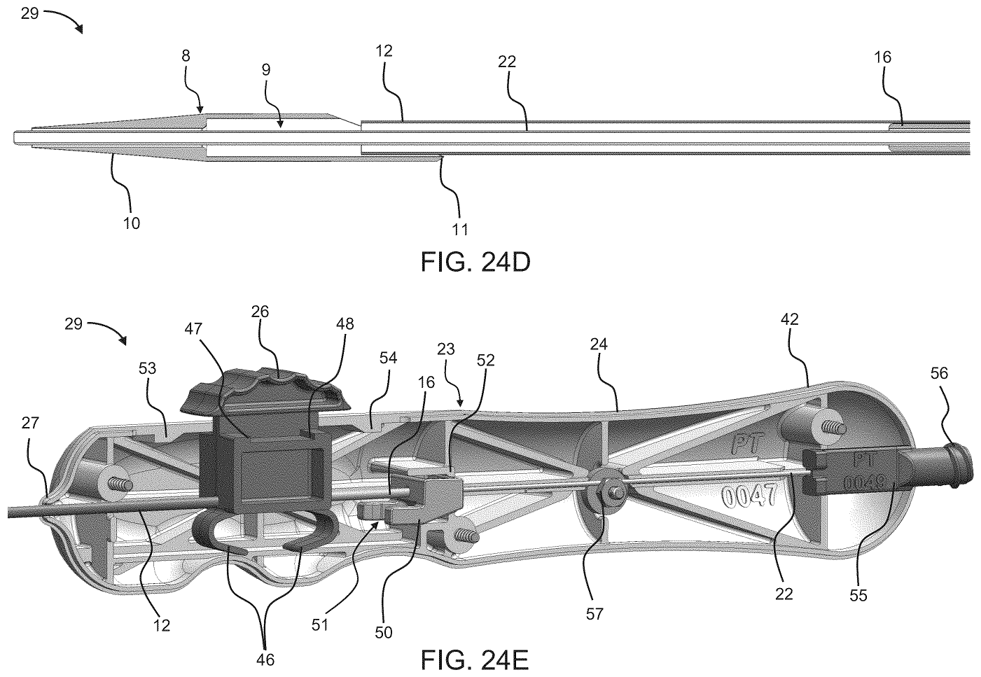

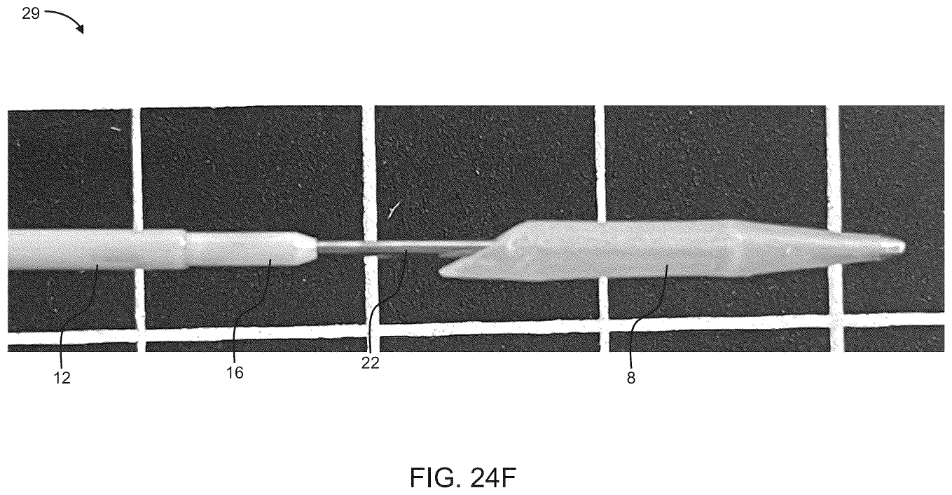

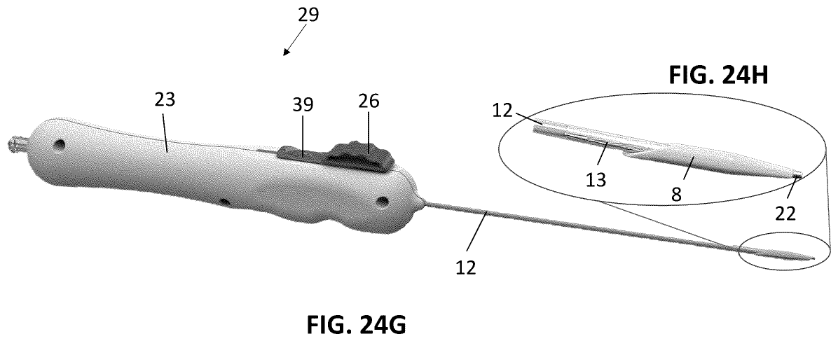

FIGS. 24A-24H depict another embodiment of a delivery system or device 29. The delivery device may be configured for percutaneous access into the arm or into another location of a patient. FIG. 24A shows a perspective view, FIG. 24B shows a cross-sectional perspective view, and FIG. 24C shows an exploded perspective view of the delivery device 29 according to this and some embodiments. Additionally, FIG. 24D shows a cross-sectional view of a distal end of the delivery device 29, and FIG. 24E shows a perspective view of the delivery device 29 with part of a handle housing of the delivery device 29 removed from view. The delivery device 29 depicted through FIGS. 24A-24H may share common elements with and may function similarly to the delivery device 29 depicted through FIGS. 21-23B; as such, common elements may share common reference numbers to indicate a general correspondence between referenced elements.

As shown in FIGS. 24A-24H, a delivery device 29 may comprise a handle 23, a control knob 26, a guidewire shaft 22, a middle shaft 16, an outer sheath 12, and a nose cone 8. The handle 23 may comprise a right housing 42 and a left housing 44, which may be secured together by mechanical fasteners, adhesive, or molded so as to snap fit or press fit together. As shown in FIG. 24A, the handle 23 may, at its proximal end, comprise a proximal handle 24, which can facilitate manipulation of the delivery device 29. Also shown in FIG. 24A, the handle 23 may, at its distal end, comprise a nose cone 27 with a thru-lumen, which can provide a guide path and support for the outer sheath 12 which is slidably disposed within the handle 23 and the nose cone 27. Also shown in FIG. 24A, the control knob 26 may be slidably constrained within a longitudinal opening of the handle 23, with a portion of the control knob 26 extending outside the handle 23 as shown for manipulation by a user. The nose cone 8 may be disposed at a distal end of the delivery device 29 and comprise features as described herein.

The perspective cross-sectional view of FIG. 24B and the exploded perspective view of FIG. 24C show additional details of the delivery device 29 shown in FIG. 24A according to some embodiments. As shown, the guidewire shaft 22 may traverse a longitudinal length of the handle 23. The guidewire shaft 22 may fluidly connect at its proximal end to a guidewire shaft connector 55 disposed partially within the handle 23 at the proximal end of the handle 23. The guidewire shaft connector 55 may be similar to the tube 25 as described herein. The guidewire shaft connector 55 may extend proximally beyond the handle 23 and terminate with a guidewire shaft connector fitting 56 that may be coupled to a valve and/or tubing for controlling bleed back when the delivery device 29 is inserted in the vasculature. The guidewire shaft connector 55, in being fluidly connected to guidewire shaft 22, may form a common lumen with the guidewire shaft 22 to allow for a guidewire 5 as described herein to be inserted into the proximal end of the guidewire shaft connector 55 (e.g., through the guidewire shaft connector fitting 56), travel through the guidewire shaft connector 55, and then travel through the guidewire shaft 22 as the guidewire 5 is translocated distally. Also shown, a guidewire shaft housing connector 57 may be disposed distal to the guidewire shaft connector fitting 56 within the handle 23, the guidewire shaft housing connector 57 configured to attach to the guidewire 22 and provide support (e.g., mechanical support) for the guidewire 22. Also shown, the delivery device 29 may further comprise a middle shaft connector 50 disposed within the handle 23 and configured to connect to a proximal end of the middle shaft 16. The delivery device 29 may further comprise a middle shaft connector stop 52, which as shown may be a feature of the handle 23 (e.g., a molded feature). Also shown, the control knob 26 may comprise a distal catch 47 and a proximal catch 48, which may interact with features of the handle 23 including, in some embodiments, a distal housing catch 53 and a proximal housing catch 54. Further, the control knob 26 may comprise spring elements 46, which may also interact with features of the handle 23.

Further connections of elements and operation of delivery device 29 according to some embodiments will be discussed next in reference to the cross-sectional view of FIG. 24D and the perspective view of FIG. 24E (which shows the delivery device 29 with left housing 44 of the delivery device 29 removed from view). As shown, the guidewire shaft 22 may extend substantially longitudinally within the handle 23 and may connect, as described above, at its proximal end to guidewire shaft connector 55. Further, the guidewire may extend distally through the handle nose cone 27 and end distally at a connection with nose cone 8. Thus, the guidewire shaft 22, in combination with the guidewire connector 55 and the nose cone 8, may form a central lumen configured to slidably accommodate a guidewire 5 as described herein (e.g., the guidewire 5 may be slid within the delivery device 29, and/or the delivery device may be slid along the guidewire 5).

The middle shaft 16 as described herein may be disposed coaxial with and slidably along the guidewire shaft 22, and as shown may have a proximal end that connects with the middle shaft connector 50 disposed within the handle 23 and a distal end that may terminate short of the nose cone 8. Thus, the middle shaft 16 and the middle shaft connector 50 may slidably move together distally/proximally along the guidewire shaft 22. The middle shaft connector 50 may have a proximal end configured to abut the middle shaft connector stop 52 when in its proximal-most position, and a distal end configured to interact with the control knob 26. In some embodiments, the middle shaft connector 50 may comprise a middle shaft connector recess 51 configured to engage the control knob 26 when the control knob 26 is slid to its proximal-most position in the handle 23. For example, the middle shaft connector recess 51 may frictionally engage the control knob 26 when the control knob 26 is slid proximally and brought within the recess. As another example, the middle shaft connector recess 51 may comprise protrusions that aid the middle shaft connector 50 in holding onto/engaging the control knob 26 when the control knob 26 is slid proximally and brought within the recess past the protrusions.

The outer sheath 12 as described herein may be disposed coaxial with and slidably along the middle shaft 16, and as shown may have a proximal end that connects with the control knob 26 and a distal end that may terminate within, partially within, or near the nose cone 8. Thus, the outer sheath 12 and control knob 26 may slidably move together distally/proximally along the middle shaft 16. The control knob 26 may comprise spring elements 46 as shown configured to provide an upward force to the control knob 26 by interaction between the spring elements 46 and an inner surface (e.g. a longitudinally-oriented inner surface) of the handle 23. The control knob 26 may also comprise features that may interact with the handle 23 and maintain the control knob 26 is desired positions distally and/or proximally. For example, the control knob 26 may comprise distal catch 47 and proximal catch 48 (which may each be in the form of a step-like edge) disposed near its distal end and its proximal end, respectively, and near where the control knob 26 protrudes through the longitudinal opening of the handle 23 as shown, and the handle 23 may comprise distal housing catch 53 and proximal housing catch 54 configured to interact with the distal catch 47 and the proximal catch 48, respectively. Further and as shown, the distal housing catch 53 and proximal housing catch 54 may be disposed adjacent a distal end and a proximal end, respectively, of the longitudinal opening of the handle 23, and may each comprise a ramp-like surface facing towards the control knob 26 and a step-like edge facing away from the control knob 26. In such embodiments and further to this example, when the control knob 26 is slid distally to its full distal position, the control knob 26 may deflect downward (e.g., inward into the handle 23) as it comes into contact and rides along the ramp-like surface of the distal housing catch 53 until the step-like edge of the distal catch 47 passes the step-like edge of the distal housing catch 53, at which point the control knob 26 can return to its more upward position within the handle 23 and the interaction between the step-like edges of the distal catch 47 and the distal housing catch 53 keep the control knob 26 locked into place. To unlock the control knob 26 from this distal position, the control knob 26 may be pushed inward towards the center of the handle 23 (opposing the upward force provided by the spring elements 46) until the step-like edge of the distal catch 47 is free of the step-like edge of the distal housing catch 53 (e.g., they are no longer overlapping longitudinally) and then the control knob 26 may be slid proximally. Locking and unlocking of the control knob 26 in its proximal-most position may be performed similarly with the corresponding proximal catch 48 and proximal housing catch 54. In some embodiments, when the control knob 26 is slid proximally into its proximal-most position, the control knob 26 may engage with the middle shaft connector recess 51 of the middle shaft connector 50, causing the middle shaft connector 50 to lock onto the control knob 26; subsequent movement/sliding of the control knob 26 may then cause the middle shaft connector 50 and the middle shaft 16 it is connected to to move together with control knob 26 and the outer sheath 12. The middle shaft connector stop 52 as described above may aid in the engagement of the middle shaft connector 50 with the control knob 26 by preventing proximal movement of the middle shaft connector 50 as the control knob 26 is moved proximally into the middle shaft connector recess 51. In some embodiments, the control knob 26 and handle 23 may comprise other features that aid in locking the control knob 26 in a desired position. In some embodiments, the distal catch 47, proximal catch 48, distal housing catch 53, and proximal housing catch 54 may comprise features different than described above but that may function similarly.

As described herein and shown also in FIG. 24D, the distal end of delivery device 29 may comprise a nose cone 8 comprising a distal taper 10, a cavity 9, and an angled proximal end 11. The distal taper 10 may be symmetric along a longitudinal length of the nose cone 8 and may comprise a longitudinal through opening; in some embodiments, the guidewire shaft 22 may attach to the nose cone 8 at the longitudinal through opening. The longitudinal through opening may comprise a diameter and/or cross-sectional area that is smaller than a diameter and/or cross-sectional area of the cavity 9. The cavity 9 may comprise a diameter and/or cross-sectional area that is greater than an outer diameter of the outer sheath 12. The nose cone 8 may comprise a proximal end that is at an angle relative to the longitudinal length of the nose cone 8. For example, and using FIG. 24D as a reference, the nose cone 8 may comprise a proximal end that is at an angle of about 30 degrees relative to the longitudinal length of the nose cone 8, although other angles may be used. In some embodiments, the nose cone 8 may comprise a proximal end that is at an angle of between about 5 degrees to about 90 degrees relative to the longitudinal length of the nose cone 8. In some embodiments, the nose cone 8 may comprise a proximal end that is at an angle of about 5 degrees, about 10 degrees, about 15 degrees, about 20 degrees, about 25 degrees, about 30 degrees, about 35 degrees, about 40 degrees, about 45 degrees, about 50 degrees, about 55 degrees, about 60 degrees, about 65 degrees, about 70 degrees, about 75 degrees, about 80 degrees, about 85 degrees, or about 90 degrees relative to the longitudinal length of the nose cone 8. In some embodiments, the angled proximal end of the nose cone 8 may allow the nose cone 8 to deflect preferentially in one direction and not in another. For example, the nose cone 8 as shown in FIG. 24D may preferentially deflect upwards (i.e., the distal end of the nose cone 8 may deflect upwards) and may not preferentially deflect downwards due to the angled proximal end. In some embodiments, the distal end of the nose cone 8 may be inhibited from movement in a direction due to the angled proximal end of the nose cone 8 substantially inhibiting movement (e.g., the proximal end of the nose cone inhibits movement due to interaction with any one of the guidewire shaft 22, the middle shaft 16, and/or the outer sheath 12). The proximal facing edge of the proximal end of nose cone 8 may comprise a taper, for example as shown in FIG. 22A. As shown, the angled proximal end 11 of the nose cone 8 may taper down to a smaller cross-sectional area in the proximal direction, i.e., it may comprise a taper opposite the distal taper 10. As such, the taper of the angled proximal end 11 may aid in retraction of the nose cone 8 as described herein. In some embodiments, the proximal end of the nose cone 8 may comprise a transverse cross-sectional shape that is circular, round, oval, oblong, pear-shaped, egg-shaped, or any symmetric or irregular shape. In some embodiments, a width of the proximal end of the nose cone 8 may be larger than a height of the proximal end of the nose cone 8 along the longitudinal axis of the nose cone 8. In some embodiments, the cavity 9 may be sized larger than an outer diameter of the outer sheath 12. In some embodiments, the cavity 9 may have a width that is greater than its height.

The guidewire shaft 22 may provide a semi-rigid semi-flexible conduit that may serve as a catheter extending from the handle 23 of the delivery device and terminate at its distal end with the nose cone 8. Thus, in use, the distal or proximal location of the nose cone 8 in the body may be directly affected by distal or proximal movement of the delivery device 29, such as by a clinician operating the delivery device 29. As described herein, the distal and/or proximal movement of the middle shaft 16 and/or the outer sheath 12 may be controlled by manipulation of the control knob 26 (e.g., by distal and/or proximal sliding of the control knob 26). The delivery device 29 may be configured for single-handed operation by a clinician/user/operator. Single-handed operation of the delivery device 29 may advantageously allow a clinician/user/operator to keep their other hand free to perform additional aspects related to the procedure. Also, single-handed operation of the delivery device 29 may reduce and/or eliminate the need for additional staff to assist with the procedure. Additionally, single-handed operation of the delivery device 29 may advantageously increase the efficiency of the procedure. Single-handed operation of the delivery device 29 may advantageously allow the clinician/user/operator to simultaneously control an ultrasound imaging probe with their other hand during the procedure.

FIG. 24F depicts a distal end of the delivery system 29 according to some embodiments. Shown in FIG. 24F are variations of the outer sheath 12 and middle shaft 16 that can aid in operation of the delivery device 29. As shown, the middle shaft 16 can have a tapered distal end to facilitate passage of the middle shaft 16 through an expanded implant 13 and/or entry into cavity 9 of the nose cone 8. Also shown, the outer sheath 12 can have a tapered distal end to facilitate passage of the outer sheath 12 through an expanded implant 13 and/or entry into cavity 9 of the nose cone 8. Furthermore, the middle shaft 16 can have an interference fit with the guidewire shaft 22, and/or the outer sheath 12 can have an interference fit with the middle shaft 16.

FIGS. 24G and 24H depict a delivery system 29 carrying an implant in its radially compressed configuration according to some embodiments. FIG. 24G also depicts a safety clip 39 removably disposed proximal to control knob 26. During shipping and until use, safety clip 39 can prevent control knob 26 from inadvertently sliding proximally. Before use, safety clip 39 can be removed, which can allow control knob 26 to move proximally as may be required to delivery implant 13 into the patient. In some embodiments and as described herein and as shown in FIG. 24H, at least a portion of the implant 13 may be disposed within the nose cone 8 and at least a portion of the implant 13 may be disposed within the outer sheath 12. FIG. 24H also depicts guidewire shaft 22 extending distally beyond the distal tip of nose cone 8. Although guidewire shaft 22 may extend distally beyond the distal tip of nose cone 8, it is understood that this is not a necessary feature of the displayed embodiment.

The delivery device 29 may be used to percutaneously deliver an intraluminal implant 13 as described herein. In some embodiments and as described herein, at least a portion of the implant 13 may be disposed within the cavity 9 of the nose cone 8. In some embodiments, at least a portion of the implant 13 may be disposed within the outer sheath 12. In some embodiments, the middle shaft 16 may abut an end (e.g., the proximal end) of the implant 13 when the implant is disposed within the delivery device 29. In some embodiments, the implant 13 may be slidably disposed over the guidewire shaft 22. In some embodiments, the implant may be disposed at least partially within the cavity 9 of the nose cone 8 while also being at least partially disposed within the outer sheath 22. In some embodiments, the implant 13 may be disposed within the delivery device 29 in a radially compressed (e.g., collapsed) configuration and packaged as a kit. In some embodiments, a kit may comprise a delivery device 29 and an implant 13. In some embodiments, the kit may further comprise a needle access tool 35 and/or a guidewire 5. In some embodiments, the delivery device 29 may be single-use. In some embodiments, the delivery device 29 may be reusable. In some embodiments, any of the components and devices described herein may be provided sterile or non-sterile.

Provided next is a description, according to some embodiments, of how the delivery device 29 described above and in reference to FIGS. 24A-24H may percutaneously deliver an implant 13 after a guidewire 5 has already been placed through a vasculature and an adjacent vasculature desired to be connected by an AVF, however the description provided may be applicable to the delivery devices and methods described previously. In some embodiments, the delivery device 29 may be provided with an implant 13 in a radially compressed (e.g., collapsed) configuration slidably disposed over the guidewire sheath 22, slidably disposed within the outer sheath 12, at least partially within the cavity 9 of the nose cone 8, and with the middle shaft 16 abutting its proximal end. Furthermore, the control knob 26 of the delivery device 29 as provided may be in its distal most position relative to the handle 23 of the delivery device 29 (and, in some embodiments, may be locked in the distal most position via the interaction between the distal catch 47 of the control knob 26 and the distal housing catch 53 of the handle 23). In some embodiments, the control knob 26 can be prevented from sliding proximally by the presence of a safety clip 39, as shown in FIG. 24G. After being slid distally over a guidewire 5 until the nose cone 8 is positioned past the desired AVF location within a lumen of the vasculature (see FIG. 4), the delivery device may be pulled back proximally to engage the nose cone 8 against the near wall of the vasculature as described herein (see FIG. 5).

For example, the delivery device may be introduced directly into a patient's vein (e.g., a perforator vein as described further herein) and the nose cone 8 may be advanced into the proximal radial artery. Delivery may occur under ultrasound guidance. The nose cone may be rotationally oriented to the near wall of the radial artery where the arterial puncture was made. The nose cone 8 may be pulled back to align with the near wall of the radial artery. A combination of tactile feedback (resistance on pull back) and ultrasound visualization may be used to localize the position on the radial artery wall.

With the delivery device 29 and its nose cone 8 in this position, the control knob 26 may be moved (e.g., slid) to its proximal most position relative to the handle 23 of the delivery device 29 to proximally retract the outer sheath 12 (see FIGS. 6A-6B). In some embodiments, this may comprise unlocking of the distal catch 47 of the control knob 26 from the distal housing catch 53 of the handle 23 before the control knob 26 can be moved proximally, and may also include locking the control knob 26 in its proximal most position via the interaction between the proximal catch 48 of the control knob 26 and the proximal housing catch 54 of the handle 23. In some cases the safety clip 39 described above is removed. The proximal retraction of the outer sheath 12 may allow at least a portion of the implant 13 to radially expand within a lumen of an adjacent vasculature (e.g., within a perforator vein) as well as at least a portion of the implant to expand within the cavity 9 of the nose cone 8. In some embodiments, the control knob 26 may engage with the middle shaft connector 50 when moved into its proximal most position within the handle 23 of the delivery device 29 and lock the middle shaft connector 50 to the control knob 26. In some cases, the control knob 26 may be initially moved proximally a short distance, .e.g., 1-2 mm, to ensure that the nose cone remains in a proper location before continuing to move the control knob 26.

With the control knob 26 kept in its proximal most position, the delivery device 29 may be advanced distally over the guidewire 5 to fully release the implant 13 from the cavity 9 of the nose cone 8 and allow the implant to fully radially expand within the lumen of the vasculature (see FIG. 8). For example, the delivery device 29 may be advanced forward at least 5 cm. The control knob 26 may be moved (e.g., slid) from its proximal most position to its distal most position relative to the handle 23 (which in some embodiments may include unlocking of the proximal catch 48 from the proximal housing catch 54 and again locking of the distal catch 47 to the distal housing catch 54), which can cause both the middle shaft 16 and the outer sheath 12 to advance distally through the radially expanded implant and engage the nose cone 8 as described herein (see FIG. 9). The delivery device 29 may be rotated (e.g., rotated about 180 degrees) to create a smooth or a substantially smooth transition between the nose cone 8 and the outer sheath 12 (e.g., eliminate any gap 14 as described herein, see FIG. 10A or 10B) and retracted proximally until it is completely removed from the body (see FIGS. 11-14).

The materials of construction of delivery device 29 can be any of the materials well known to be used for catheter construction such as PEEK, HDPE, PeBax, nylon, PTFE, combinations thereof, and others. The diameter and length of delivery device 29 can be suited to the particular application. In some embodiments, the profile of nose cone 8 may be between 6 Fr. and 9 Fr. for an implant 13 diameter of between 3 mm and 6 mm. In some embodiments, the distance between handle 23 and nose cone 8 may be between about 15 cm and about 30 cm. Various lengths and diameters of delivery device 29 can be used in various embodiments to satisfy the needs of the particular application. Nose cone 8 can be constructed from materials well known to be suitable for catheters such as, for example, PEEK, HDPE and Polypropylene. In some embodiments, nose cone 8 may include echogenic features to aid in ultrasound visualization.

In some embodiments, the delivery device 29 may comprise a mesh or other wrapping disposed around the implant 13 to maintain the implant 13 in a radially compressed (e.g., elastically constrained) configuration. In such embodiments, the implant 13 may be released from its radially compressed configuration and expanded into its radially expanded configuration by pulling a thread or wire of the mesh or wrapping configured to tear the mesh or wrapping, thus releasing the implant 13. Furthermore, in such embodiments the delivery device 29 may not require an outer sheath 12 or a middle shaft 16.

Returning to the simplified representation of a portion of the vasculature of the human arm shown FIG. 1, the location 7 that is a potential area to create an anastomotic connection may be where a radial artery is adjacent to a branching point between a brachial vein and a radial vein in the proximity of a perforator vein. For example, as depicted in FIG. 1, artery 4 may comprise a radial artery, deep vein 3 may comprise a brachial vein and/or a radial vein (e.g., to the left of location 7 the vein may be the brachial vein, and to the right of location 7 the vein may be the radial vein), perforator vein 2 may comprise a perforator vein, and superficial vein 1 may comprise a cephalic vein. Prior to implantation of implant 13, blood flow may be as follows as described relative to FIG. 1: blood flow in the radial artery (e.g., artery 4) may be from left to right; blood flow in the brachial vein and the radial vein (e.g., deep vein 3) may be from right to left; blood flow in the perforator vein (e.g., perforator vein 2) may be from the brachial vein and/or the radial vein and diagonally upwards and to the left to the cephalic vein; and blood flow in the cephalic vein (e.g., superficial vein 1) may be from right to left.

In some embodiments, before the guidewire 5 may be placed (as shown in FIGS. 2A-2D), a few preparatory steps can be taken. Regional anesthesia (e.g., axillary or clavicular block) can be administered to the patient. The patient's arm can be positioned on an arm board at 90° abduction. And a circumferential skin prep can be provided up to the axilla.

Returning to FIGS. 2A-2D, in some embodiments the guidewire 5 may be placed percutaneously through a wall of the cephalic vein (e.g., superficial vein 1), through the cubital perforating vein (e.g., perforator vein 2), through the brachial vein or the radial vein (e.g., deep vein 3), through a wall of the brachial vein or the radial vein (e.g., deep vein 3), through any interstitial tissues between the brachial vein or the radial vein and the radial artery (e.g., artery 4), through a wall of the radial artery (e.g., artery 4), and into the radial artery (e.g., artery 4). In some embodiments, the steps of placing the guidewire 5 can include the following: first, a sterile elastic tourniquet can be applied to the upper arm to increase venodilation. Second, the patient's skin can be marked about 2 cm cranial to the interface of the cubital perforating vein (e.g., perforator vein 2) and proximal radial artery (e.g., artery 4). This interface can be found using ultrasound in a long-axis view along the cubital perforating vein. Third, under ultrasound guidance, a needle (e.g., a 21 g echogenic needle) can access the cephalic vein (e.g., superficial vein 1) at least about 2 cm cranial from the interface of the cubital perforating vein (e.g., perforator vein 2) and proximal radial artery (e.g., artery 4). The needle can then be guided translumenally to the septum between the cubital perforating vein (e.g., perforator vein 2) and the proximal radial artery (e.g., artery 4). Fourth, under transverse ultrasound view, the needle can be advanced from the cubital perforating vein (e.g., perforator vein 2) into the proximal radial artery (e.g., artery 4). Preferably, the angle of approach for advancing the needle into the proximal radial artery is directly anterior (i.e., in the 12:00 o'clock position).

Returning to FIG. 15A, in some embodiments the implant 13 may be implanted with the distal implant segment 18 (which may also be referred to herein as the arterial implant segment) positioned within the radial artery (e.g., artery 4) and the proximal segment 19 (which may also be referred to herein as the venous implant segment) extending through the brachial vein or the radial vein (e.g., deep vein 3) and the cubital perforating vein (e.g., perforator vein 2). In some cases, the cubital perforating vein (e.g., perforator vein 2) into which the implant 13 is implanted has a diameter of at least about 2.5 mm and a length of at least about 10 mm. In some cases, the proximal radial artery (e.g., artery 4) into which the implant 13 is implanted has a diameter of at least about 2 mm. And in some cases, the distance between the proximal radial artery (e.g., artery 4) and the cubital perforating vein (e.g., perforator vein 2) is less than about 3 mm. Such a configuration creates a proximal radial artery to cubital perforating vein arteriovenous fistula, which can be advantageous, e.g., in patients who require hemodialysis. An arteriovenous fistula according to some embodiments may in some cases be well suited for patients having a minimum proximal radial artery diameter of about 2 mm and a minimum cubital perforating vein diameter of about 2.5 mm.

After implantation of implant 13 as such, blood flow may be as follows as described relative to FIG. 15B: blood flow in the radial artery (e.g., artery 4) may be antegrade from left to right (shown as the first arrow in artery 4 to the left of or upstream of the opening 60) and enter the side opening or port 60 (e.g., the side opening or port between the proximal implant segment 19 and the distal implant segment 18) of the implant 13 as shown and (i) flow through the proximal end of the distal implant segment 18 and out the distal end of the distal implant segment 18 to continue through the artery (shown as the second arrow in artery 4 downstream of the distal implant segment 18), and (ii) flow through the distal end of the proximal implant segment 19 and out the proximal end of the proximal implant segment 19 to flow into the cubital perforating vein (e.g., perforator vein 2) and into the cephalic vein (e.g., superficial vein 1) (shown as the arrow in vein 2). In some embodiments and with continued reference to FIG. 15B, after implantation of implant 13 as described above, blood flow through the brachial vein and/or the radial vein (e.g., deep vein 3) may be at least partially blocked or completely blocked by the proximal implant segment 19. In some embodiments, the proximal segment 19 blocking blood flow through the brachial vein and/or the radial vein (e.g., deep vein 3) may advantageously send more blood through the cephalic vein (e.g., superficial vein 1) and further enhance the development of the cephalic vein for use in hemodialysis. In some embodiments, the proximal segment 19 of the implant 13 may advantageously send blood from the radial artery (e.g., artery 4) directly into the cubital perforating vein (e.g., perforator vein 2) and/or the cephalic vein (e.g., superficial vein 1) and bypass one or more branch points of the brachial vein and/or the radial vein (e.g., deep vein 3).

In some embodiments and with continued reference to FIG. 15B, after implantation of implant 13 as described above, blood flow through the cephalic vein (e.g., superficial vein 1) may be from right to left (as shown by the arrow in vein 1), and may include venous blood as well as arterial blood as provided by the implant 13. Arterial blood may flow through the proximal implant segment 19 of the implant 13 due to the pressure differential between the artery 4 and the perforator vein 2 and/or superficial vein 1 (i.e., arterial blood is under more pressure than venous blood, and fluid will tend to flow from high pressure to low pressure). The flow of arterial blood in the cephalic vein (e.g., superficial vein 1) due to the implant 13 may advantageously cause the cephalic vein to increase in at least one of its size (e.g., diameter), thickness, or blood flow rate. For example, the flow of arterial blood in the cephalic vein (e.g., superficial vein 1) due to the implant 13 may advantageously cause the cephalic vein to increase in diameter to at least about 4 mm, at least about 5 mm, at least about 6 mm, at least about 7 mm, at least about 8 mm, or at least about 9 mm. In another example, the flow of arterial blood in the cephalic vein (e.g., superficial vein 1) due to the implant 13 may advantageously cause the blood flow in the cephalic vein (e.g., superficial vein 1) to be at least about 400 ml/min, at least about 500 ml/min, at least about 600 ml/min, at least about 700 ml/min, at least about 800 ml/min, at least about 900 ml/min, or at least about 1000 ml/min. In some embodiments, the implant 13 may lead the cephalic vein (e.g., superficial vein 1) to develop into a point of single access for hemodialysis (e.g., two needles, one for outflow and the other for return flow, in the same vein). In some embodiments, the implant 13 may lead the cephalic vein (e.g., superficial vein 1) to develop a diameter of at least about 6 mm and a blood flow rate of at least about 600 cc/min.

With reference to FIG. 15C, in some embodiments the implant 13 is configured to allow retrograde blood flow in the artery 4 to (i) flow through the distal end of the distal implant segment 18 and out the proximal end of the distal implant segment 18, and (ii) flow through the distal end of the proximal implant segment 19 and out the proximal end of the proximal implant segment 19 (as shown by the arrow in vein 2). For example, blood flow in the radial artery (e.g., artery 4) may be from right to left (e.g., retrograde flow) and enter the distal end of the distal implant segment (as shown by the arrow to the right of the distal implant segment 18 in artery 4), out the proximal end of the distal implant segment 18 (as shown by the arrow to the left of the distal implant segment 18), into the distal end of the proximal implant segment 19 (as shown by the arrow to the left of the distal implant segment 18), and out the proximal end of the proximal implant segment 19 to flow into the cubital perforating vein (e.g., perforator vein 2) and/or the cephalic vein (e.g., superficial vein 1) (as shown by the arrows in the vein 2 and the vein 1). The implant 13 can be configured to maintain fistula blood flow into the perforator vein 2 and/or superficial vein 1 during retrograde blood flow in the artery 4 (from right to left in FIG. 15C) and/or during antegrade blood flow in the artery 4 (from left to right in FIG. 15B). In other words, the implant 13 can be configured for blood flow to enter the distal end of the proximal implant segment 19 and exit the proximal end of the proximal implant segment 19 when blood flow (i) enters the side opening or port 60 and/or when blood flow (ii) enters the distal end of the distal implant segment 18 and exits the proximal end of the distal implant segment 18. In some cases, retrograde flow in the artery 4 is promoted by the implantation of the implant 13 by causing blood to flow from the brachial artery to the ulnar artery, the palmar arch and retrograde into the radial artery. In some cases, implantation of the implant 13 may cause the radial artery upstream of the implant 13 to spasm, reducing or prevent antegrade flow in the radial artery. This reduction or prevention of antegrade flow in the radial artery may resolve itself such that antegrade flow resumes, as shown in FIG. 15B.

With reference to FIG. 15D, in some embodiments the implantation of the implant 13 as shown may result in a back-and-forth flow of blood in the artery 4. Blood flow in the radial artery (e.g., artery 4) may be antegrade from left to right (shown as the first arrow in artery 4 to the left of or upstream of the opening 60) and enter the side opening or port 60 (e.g., the side opening or port between the proximal implant segment 19 and the distal implant segment 18) of the implant 13 as shown and flow through the proximal end of the distal implant segment 18 and out the distal end of the distal implant segment 18. Based on the heart cycle, blood exiting the distal end of the distal implant segment 18 may flow both antegrade and retrograde (to the right and to the left, shown by the double headed arrow), e.g., antegrade during systole and retrograde during diastole. Retrograde blood flow in the artery 4 can flow through the distal end of the proximal implant segment 19 and out the proximal end of the proximal implant segment 19 to flow into the cubital perforating vein (e.g., perforator vein 2) and/or the cephalic vein (e.g., superficial vein 1) (as shown by the arrows in the vein 2 and the vein 1). In some cases, implantation of the implant 13 may result in any or all of the hemodynamic conditions described above. For example, after initial implantation of the implant 13, blood may flow through antegrade through the artery 4 and into the implant 13 according to FIG. 15B. After a period of time (such as a few weeks), the implant 13 may cause remodeling and the hemodynamic conditions may change to provide for retrograde flow through the artery 4 as described with respect to FIG. 15C. After a further period of time (e.g., several months after initial implantation), remodeling due to implantation may be complete resulting in the back-and-forth flow described with respect to FIG. 15D.

In further reference to FIGS. 15A-15D and as described herein, the proximal implant segment 19 may be angled relative to the distal implant segment 18. Said another way and as described herein, in some embodiments, an axis (e.g., longitudinal axis) of the proximal implant segment 19 may be angled relative to an axis (e.g., longitudinal axis) of the distal implant segment 18. The axis of the proximal implant segment may be angled relative to the axis of the distal implant segment by between about 0 and 90 degrees. In some embodiments, the axis of the proximal implant segment may be angled relative to the axis of the distal implant segment by about 5 degrees, about 10 degrees, about 15 degrees, about 20 degrees, about 25 degrees, about 30 degrees, about 35 degrees, about 40 degrees, about 45 degrees, about 50 degrees, about 55 degrees, about 60 degrees, about 65 degrees, about 70 degrees, about 75 degrees, about 80 degrees, about 85 degrees, or about 90 degrees.

In further reference to FIGS. 15A-15D as well as to at least FIGS. 16C, 17A, 17B, 17C, 18A, 18B, 18C, 18D, 19A, 19B, 20A, and 20B and as described previously herein, the implant 13 may comprise a side opening or port 60 disposed between the proximal implant segment 19 and the distal implant segment 18. The side opening or port 60 may be disposed between the distal end of the proximal implant segment 19 and the proximal end of the distal implant segment 18. As shown in at least some of the figures mentioned above, the side opening or port 60 may be formed by the continuous strut and/or ring 21 of the proximal implant segment 19, the connector struts 20, and the struts comprising the proximal end of the distal implant segment 18. In some embodiments, the distal implant segment 18 may be positioned downstream of the distal end of the proximal implant segment 19 (e.g., downstream in regard to the direction of arterial blood flow as shown).

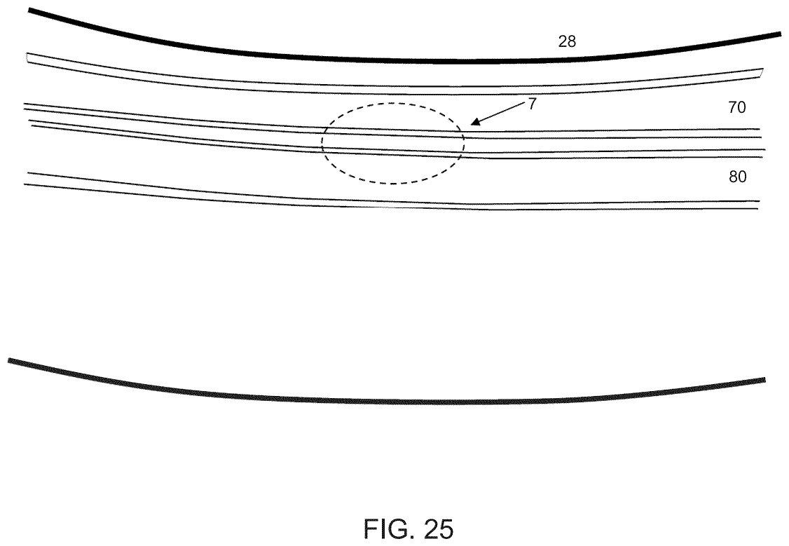

FIG. 25 depicts a simplified representation of a portion of the human vasculature indicating a potential location 7 to create an anastomotic connection (e.g., AVF) between two blood vessels, e.g., an artery 70 and a vein 80, that underly the dermal surface 28 according to some embodiments.

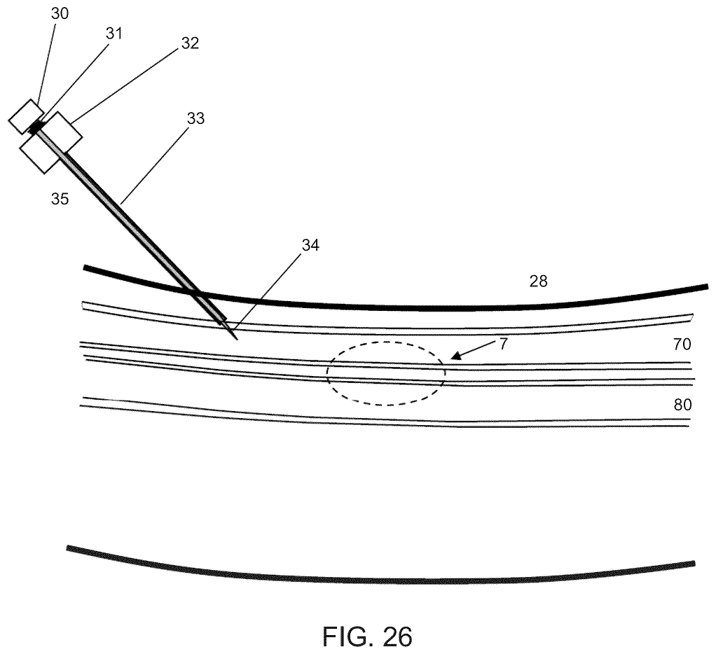

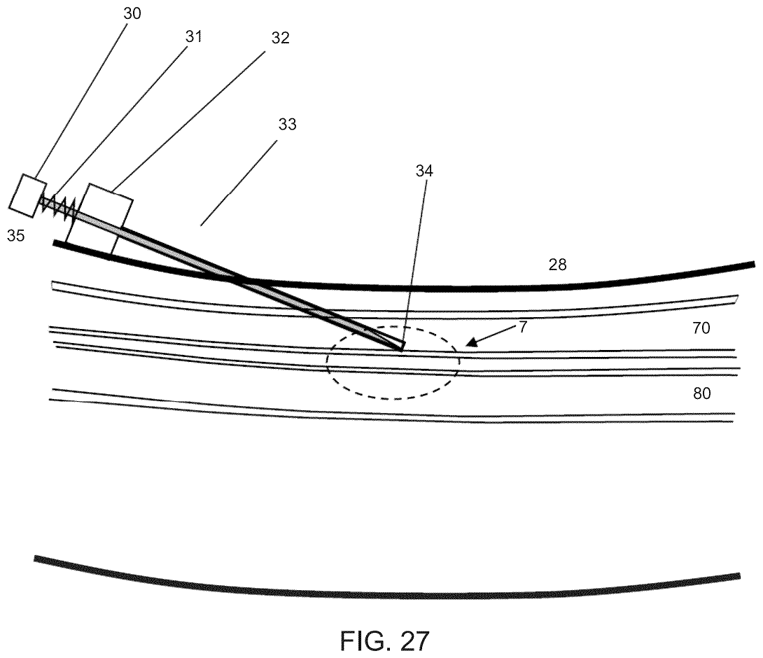

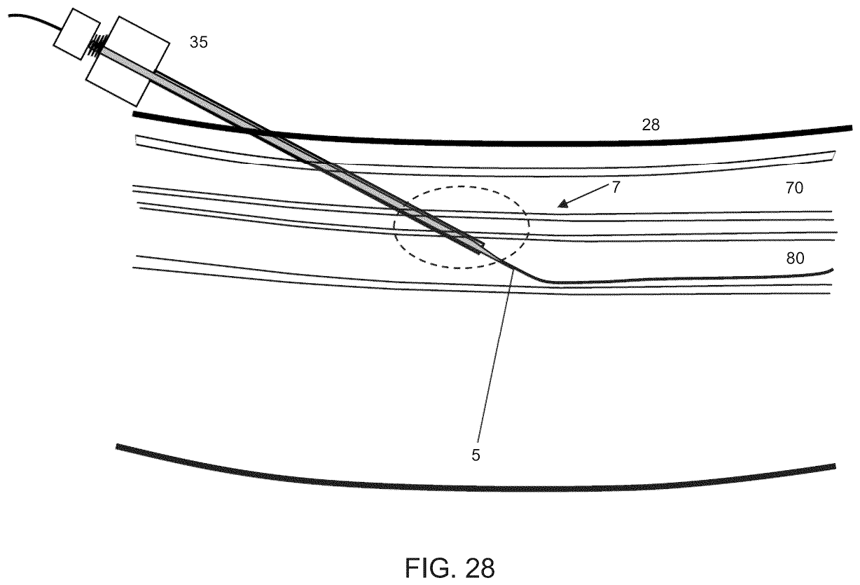

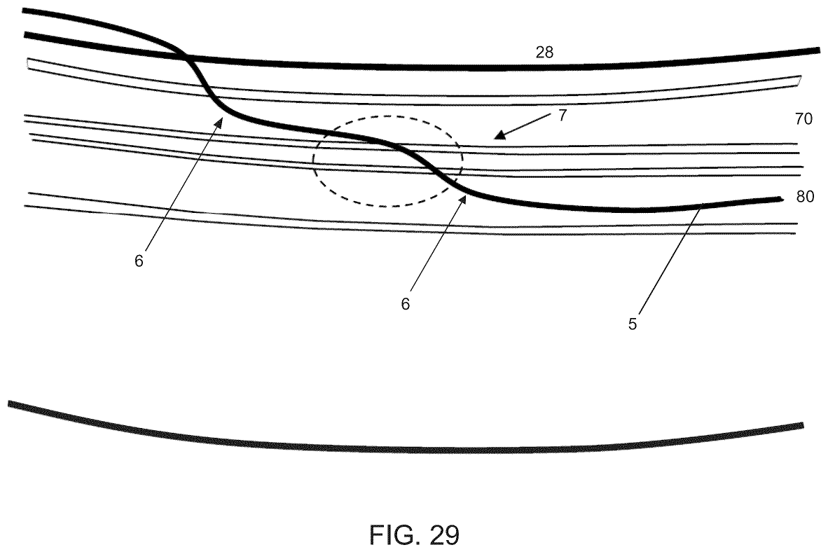

FIGS. 26-29 depict a method of percutaneously introducing an endovascular guidewire 5 according to some embodiments. The method shown and described through FIGS. 26-29 may comprise similar steps and/or aspects to the method previously described through FIGS. 2A-2D herein. As shown in FIGS. 26-29, the guidewire 5 may be percutaneously introduced by the needle access tool 35 as described herein. The needle access tool 35 can include a hollow needle with proximal port 30 and distal sharpened tip 34 slidably disposed within sheath 33. Sheath 33 may be connected to hub 32 that may comprise a compression element, such as compression spring 31 placed between port 30 and hub 32. When port 30 is depressed, needle tip 34 may be exposed distally of the distal end of sheath 33 and be able to puncture tissue such as skin and blood vessels. When port 30 is not depressed, spring 31 may decompress and move needle tip 34 proximally so that it is not exposed. In this configuration, the needle access tool 35 can navigate the vasculature with reduced risk of inadvertent punctures and trauma to the vasculature or other tissues. Using this feature of the needle access tool 35 and appropriate imaging techniques, such as transdermal ultrasound, the needle access tool 35 may be first introduced into artery 70 as shown in FIG. 26. With needle tip 34 retracted within sheath 33, the needle access tool 35 may be navigated to location 7 using appropriate imaging as shown in FIG. 27. While at location 7, the proximal port 30 may be actuated (e.g., depressed) to expose needle tip 34 and the needle access tool 35 may then be advanced to penetrate the vascular walls and the or any interstitial tissues between artery 70 and vein 80 until the distal end of sheath 33 enters the lumen of vein 80. While maintaining this position, guidewire 5 may be introduced into proximal port 30 and advanced through the needle access tool 35 until the distal end of guidewire 5 exits the distal end of the needle access tool 35 and enters the lumen of vein 80 as shown in FIG. 28. FIG. 29 shows guidewire 5 with curvatures 6 which may form when guidewire 5 conforms to the particular vascular anatomy after removal of the needle access tool 35.

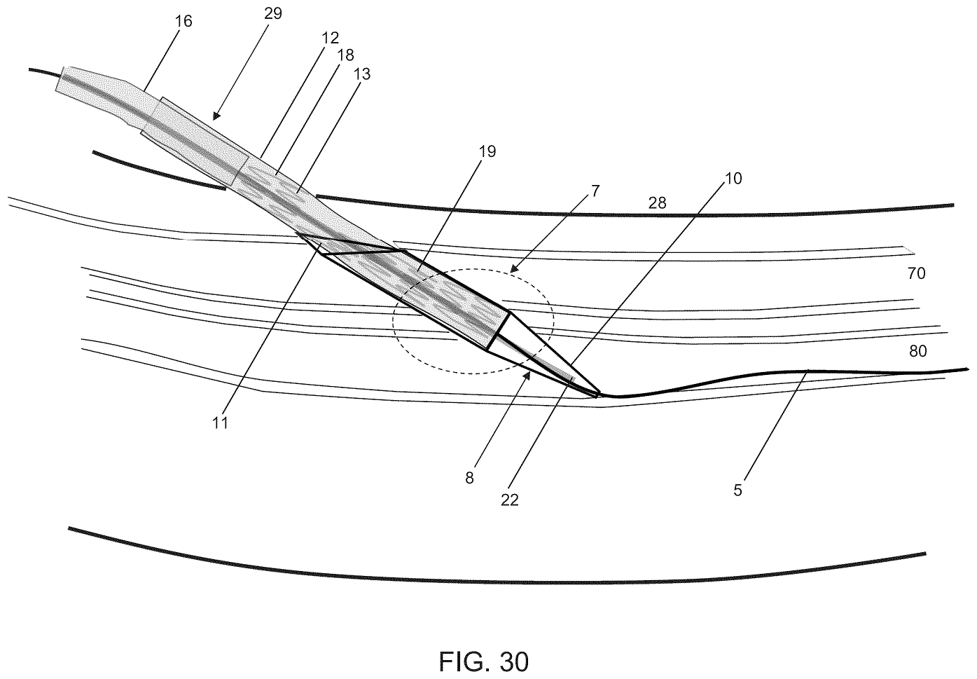

FIGS. 30-40 depict a method of percutaneously implanting an endovascular implant 13 with a delivery device 29 according to some embodiments. FIG. 30 depicts the delivery device 29 after being placed over guidewire 5 and slid along the guidewire 5 distally to cause its distal end (e.g., the nose cone 8 at the distal end of the delivery device 29) to go through the dermal surface 28, through the artery 70, through the or any interstitial space between the artery 70 and the vein 80, and into the vein 80 (e.g., into the lumen of vein 80). As shown, implant 13 may be disposed within the delivery device 29 in a radially compressed configuration. In some embodiments, the proximal implant segment 19 as described herein may be disposed within the distal end of the delivery device 29 in a radially compressed configuration and the distal implant segment 18 may be disposed within the distal end of the delivery device 29 in a radially compressed configuration, however as shown here and opposite the method of implantation described through FIGS. 3-15, the distal implant segment 18 may be oriented more proximal of the distal end of the delivery device 29 than the proximal implant segment 19 (e.g., the implant 13 is reversed relative to its orientation as described through FIGS. 3-15). Thus, as described relative to FIGS. 30-40, the distal implant segment 18 will be referred to as the arterial implant segment 18, and the proximal implant segment 19 will be referred to as the venous implant segment 19; however, when discussing the distal and proximal ends of the implant segments, the convention used thus far herein will remain. Returning to FIG. 30, as shown the implant 13 may be disposed over guidewire shaft 22 and radially compressed within the outer sheath 12, with the venous implant segment 19 disposed at least partially within the nose cone 8 (e.g., within the cavity 9 of the nose cone 8) and the arterial implant segment 18 (i.e., the distal end of the arterial implant segment 18 keeping with prior convention) abutting the distal end of the middle shaft 16.

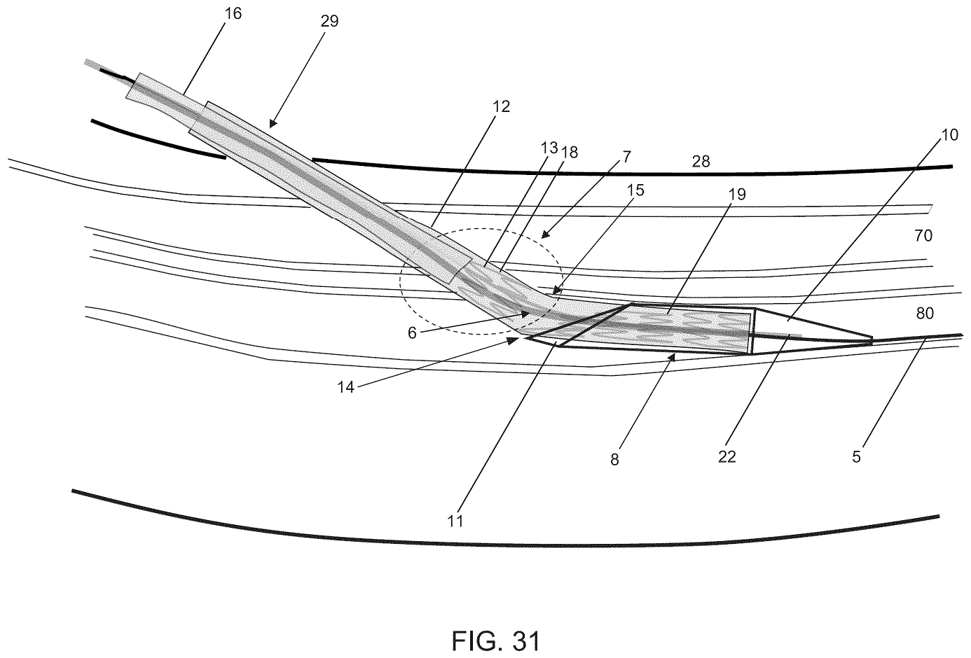

FIG. 31 depicts the nose cone 8 of delivery device 29 advanced across the AVF location 7 and within vein 80. Also shown is the gap 14 which may form when the delivery device 29 follows guidewire curvature 6 and the nose cone 8 and the outer sheath 12 are no longer coaxial. The gap 14 can be defined in some embodiments as an open space between the proximal opening of the nose cone 8 (e.g., the proximal opening of cavity 9 of nose cone 8) and a sidewall of the outer sheath 12 as it enters the proximal opening (e.g., cavity 9) of the nose cone 8. In some embodiments, the gap 14 defines about, at least about, or no more than about 5%, 10%, 15%, 20%, 25%, 30%, 35%, 40%, 45%, 50%, or more or less in length and/or diameter of the respective length and/or diameter of the proximal opening (e.g., cavity 9) of the nose cone 8, or ranges including any two of the foregoing values. Of note and as shown, to form the gap 14 the nose cone 8 may need to be oriented as shown in FIG. 31, e.g., with its longer trailing proximal end (created due to the proximal end being at an angle with the longitudinal length of the nose cone in some embodiments as shown) being oriented on the outside of the curve 6. Further shown, the angle 15 between the central (e.g., longitudinal) axis of nose cone 8 and the central (e.g., longitudinal) axis of outer sheath 12 may form when the nose cone 8 at the distal end of delivery device 29 is in the curved configuration as shown in FIG. 31 and oriented the same as way as to form gap 14. In some embodiments, the angle 15 can be, for example, about, at least about, or no more than about 5, 10, 15, 20, 25, 30, 35, 40, 45 degrees, or more or less, and ranges including any two of the foregoing values. To facilitate greater flexibility between nose cone 8 and outer sheath 12, nose cone 8 may have a slit in the wall that forms cavity 9. In some embodiments, to facilitate greater flexibility between nose cone 8 and outer sheath 12, cavity 9 of the nose cone 8 may be oversized relative to the outer diameter of the outer sheath 12. In some embodiments, the curvature 6 of guidewire 5 may be used to form angle 15 and gap 14. Alternative means of forming angle 15 and gap 14 can be utilized. An alternative embodiment could be, for example, to use one, two, or more pull wires to deflect the distal end of delivery device 29 so that gap 14 is formed. A wide variety of steerable and/or deflectable elements can be utilized depending on the desired clinical result. In some embodiments, a gap 14 may not be formed and/or be required, such as when the nose cone 8 is configured as described and illustrated with respect to FIG. 22B.

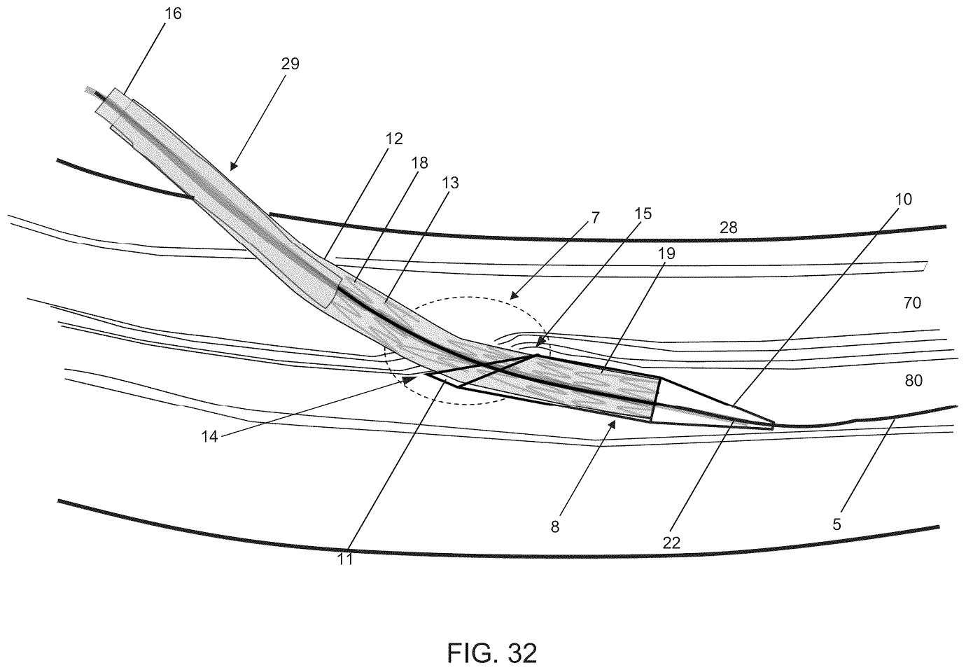

FIG. 32 depicts the nose cone 8 engaging the near wall of the vein 80 after delivery device 29 has been pulled proximally from its location in FIG. 31 according to some embodiments. The engagement between nose cone 8 (e.g., the proximal end of nose cone 8) and the near wall of the vein 80 may be due to the gap 14 which formed when delivery device 29 was urged into a curved configuration with the angled proximal end 11 (e.g., the longer trailing proximal end) on the outside of the curvature. In some embodiments, no gap 14 is required for the engagement between nose cone 8 (e.g., the angled proximal end 11 of nose cone 8) and the near wall of the vein 80. For example, the angled proximal end 11 of the nose cone 8 may engage the near wall of the vein 80 upon the delivery device 29 being pulled proximally. Also depicted is the deformation of the anatomy at AVF location 7, which is a result of the apposition forces between the near wall of the vein 80 and the angled proximal end 11 of the nose cone 8. This deformation of the anatomy at AVF location 7 may be visualized by ultrasound and used to verify proper tissue engagement and/or implant placement before delivery.

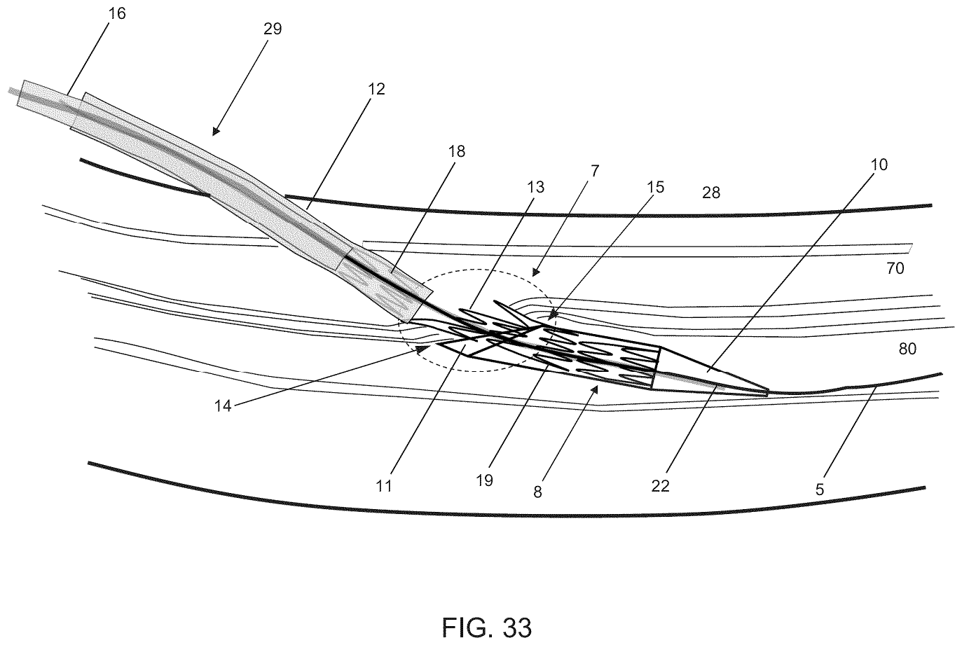

FIG. 33 depicts an initial step of the first stage of delivery of the radially compressed (e.g., elastically constrained) implant 13 according to some embodiments. While nose cone 8 is held in apposition against the near wall of vein 80, the outer sheath 12 may be retracted proximally so that the radially compressed implant 13 is allowed to expand with the precise location defined due to the engagement between nose cone 8 and the near wall of the vein 80. The distal end of the middle shaft 16 may be held fixed during retraction of the outer sheath 12 so that the radially compressed implant 13 does not slip proximally during the retraction of the outer sheath 12. Also depicted is the venous implant segment 19 being held at least partially constrained by cavity 9 of the nose cone 8, which can occur upon retraction of the outer sheath 12 (e.g., the outer sheath 12 no longer keeps the venous implant segment 19 radially compressed, so the venous implant segment 19 expands into the cavity 9). In some embodiments and also depicted is a portion of the venous implant segment 19 (e.g., the distal end but here oriented proximally) radially expanding at AVF location 7 due to the radial stiffness of the implant 13. Also depicted is the arterial implant segment 18 still radially compressed within the outer sheath 12. To retract the outer sheath 12, the control knob 26 of the delivery device 29 may be slid proximally from its distal most position with initial delivery of the implant 13 while the handle 23 of the delivery device 29 is held in position to maintain the nose cone 8 in apposition against the near wall of vein 80. In some embodiments, the middle shaft 16 may be held fixed during retraction of the outer sheath 12 by the middle shaft connector 50 being prevented from moving proximally by its interaction with middle shaft connector stop 52 of the delivery device 29.

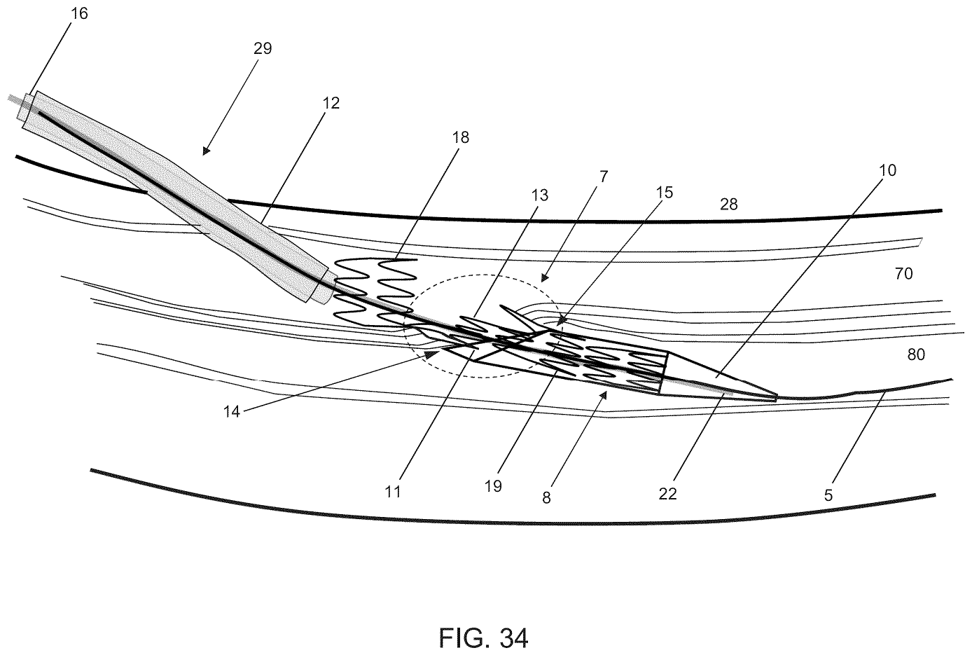

FIG. 34 depicts the continuing delivery of the radially compressed (e.g., elastically constrained) implant 13 with further retraction of the outer sheath 12 until the arterial implant segment 18 is fully released from the outer sheath 12 according to some embodiments. As shown, upon full retraction of the outer sheath 12, the arterial implant segment 18 may radially expand within the artery 70. Upon radially expanding within the artery 70, the arterial implant segment 18 may radially engage the wall of the artery 70. Also shown and in some embodiments, upon full retraction of the outer sheath 12, the arterial implant segment 18 may form an angle relative to the venous implant segment as described herein (e.g., the longitudinal axis of the arterial implant segment 18 may form an angle relative to the longitudinal axis of the venous implant segment 19). To further retract the outer sheath 12, the control knob 26 of the delivery device 29 may be slid further proximally to its proximal most position while the handle 23 of the delivery device 29 is held in position to maintain the nose cone 8 in apposition against the near wall of vein 80. In some embodiments, the middle shaft 16 may be held fixed during retraction of the outer sheath 12 by the middle shaft connector 50 being prevented from moving proximally by its interaction with middle shaft connector stop 52 of the delivery device 29. It is to be understood that while the actions of FIG. 33 and FIG. 34 have been described discretely, in practice the actions depicted and described in FIG. 33 and FIG. 34 may follow one another in a smooth fashion. For example, the outer sheath 12 may be retracted fully in one motion by movement (e.g., sliding) of the control knob 26 of the delivery device 29 from its initially distal most position to its proximal most position, which may fully release the implant 13 from the outer sheath 12 in the one motion.

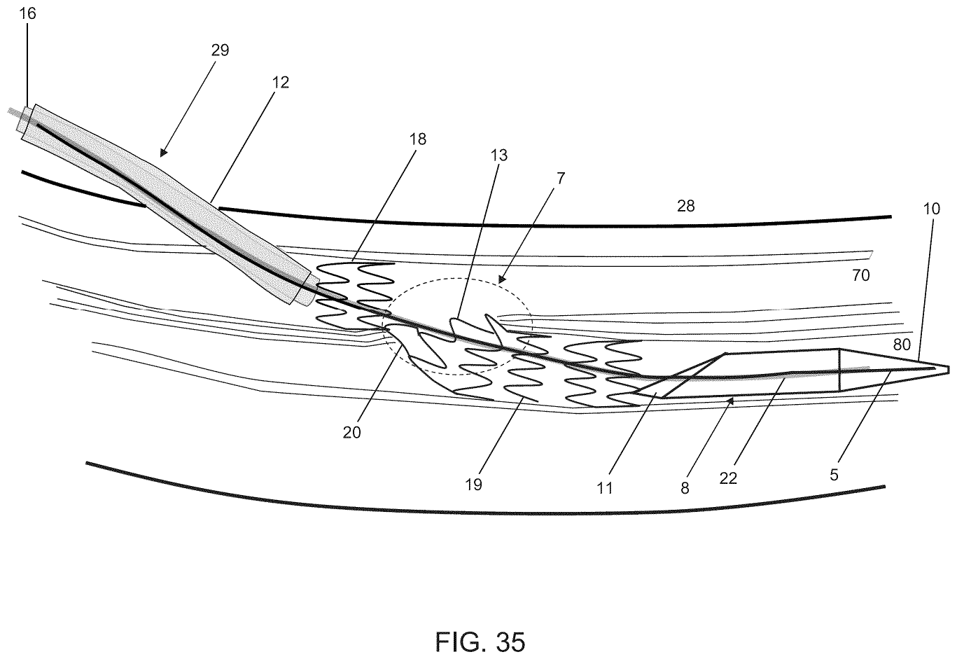

FIG. 35 depicts the delivery of the radially compressed (e.g., elastically constrained) venous implant segment 19 according to some embodiments. As shown, when nose cone 8 is advanced distally by advancing guidewire shaft 22 distally (e.g., by advancing the delivery device 29 distally), the venous implant segment 19 may be held in place by connector struts 20 connected to the arterial implant segment 18 and is thus slidably released from cavity 9 of the nose cone 8. Furthermore and as shown, when the venous implant segment 19 is released from cavity 9, it may radially expand within the vein 80. Upon radially expanding within the vein 80, the venous implant segment 19 may radially engage the wall of the vein 80. Also as shown, the arterial implant segment 18 may provide a means of securing the most distal portion of the distal edge (as oriented here the most proximal portion of the proximal edge) of the venous implant segment 19 so that it does not encroach into the luminal space of artery 70. Additionally as shown, upon radially expanding, the distal end (as oriented here the proximal end) of the venous implant segment 19 may radially expand at the far wall of the artery 70 and form a fluidic seal with the far wall of the artery 70. The arterial implant segment 18 may also provide radial support for the artery 70 to ensure patency and sufficient blood flow in the artery 70 after implantation of the implant 13.

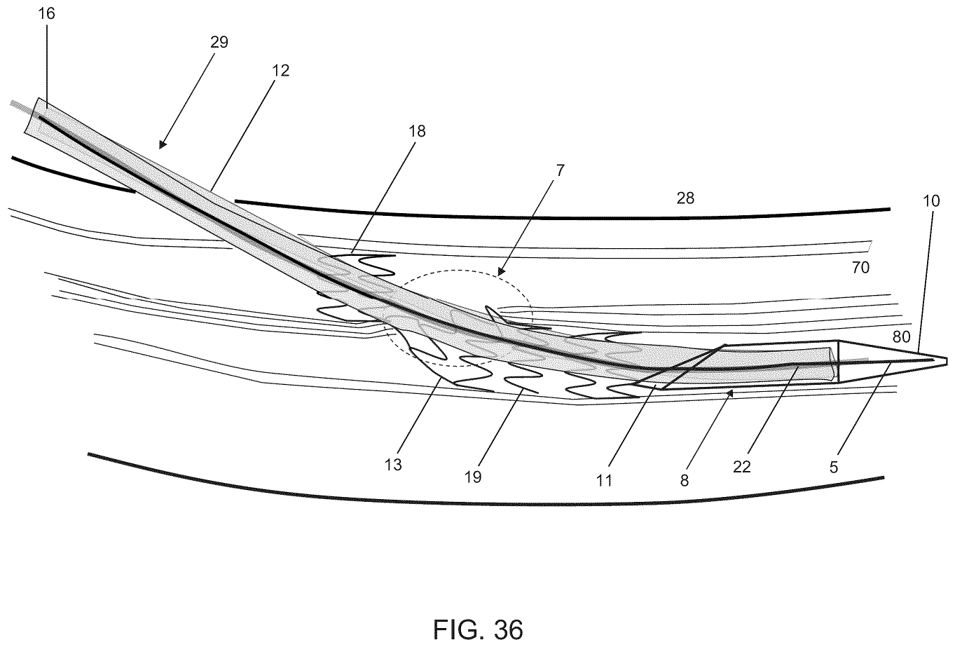

FIG. 36 depicts the initial step of removal of the delivery device 29 with the advancement of the outer sheath 12 and the middle shaft 16 distally through the delivered (e.g., radially expanded) implant 13 and into cavity 9 of the nose cone 8 according to some embodiments. In some embodiments, the middle shaft 16 may lead the outer sheath 12 during this advancement step to facilitate reliable engagement of the outer sheath 12 into the cavity 9 without the outer sheath 12 catching the proximal end 11 of the nose cone 8. To move/advance the outer sheath 12 distally, the control knob 26 of the delivery device 29 may be moved (e.g., slid) distally to its distal most position within handle 23 while the handle 23 of the delivery device 29 is maintained in position. Furthermore and in some embodiments, the middle shaft 16 may be made to lead and advance with the outer shaft 12 by the middle shaft connector 50 engaging with the control knob 26 after the control knob 26 has been moved (e.g., slid) into its proximal most position from an earlier step in the delivery process and the middle shaft connector 50 moving distally with the control knob 26 upon the control knob 26 being moved distally.

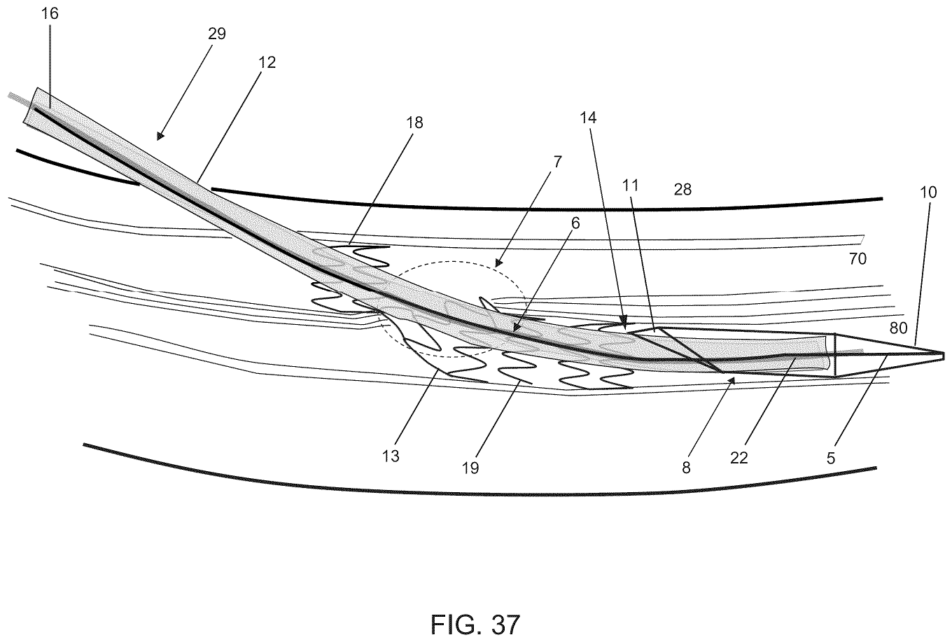

FIG. 37 depicts a continuation of the removal of the delivery device 29 with the rotation of the delivery system 29 around its axis of, e.g., approximately 180 degrees such that the proximal portion of the angled proximal end 11 of the nose cone 8 is on the inside of curvature 6 according to some embodiments. In this orientation, the gap 14 may be minimized, eliminated, or substantially eliminated and there may be flush contact between the proximal tapered end 11 and the outer sheath 12. This low-profile configuration may facilitate removal of the nose cone 8 without engagement of the delivered implant 13 or any of the anatomical features near AVF location 7. In some embodiments, the engagement between the outer sheath 12 and the proximal end of the nose cone 8 may follow as shown and described relative to FIG. 10B herein, e.g., the proximal tapered end of the nose cone 8 may be received within the lumen of the outer sheath 12 upon advancement of the outer sheath 12 distally, creating a substantially smooth transition between the outer sheath 12 and the proximal end of the nose cone 8 and thus aiding in removal of the delivery device 29.

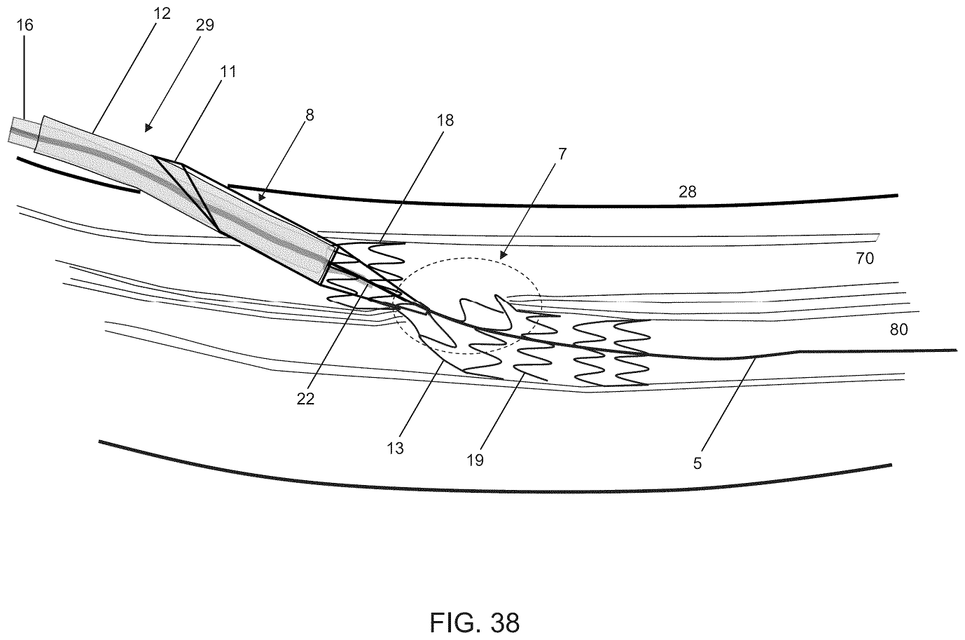

FIG. 38 depicts a continuation of the removal of the delivery device 29 according to some embodiments. As shown, with proximal retraction of the delivery device 29, the nose cone 8 has passed through the majority of the implant 13 without interference. The implant 13 may have unconstrained (e.g., radially expanded) delivered internal dimensions that are greater than the outer dimension of nose cone 8 so that nose cone 8 does not experience excessive resistance or interference with the implant 13 upon removal through implant 13. In some embodiments and as described relative to FIG. 13 herein, if the gap 14 has formed again during retraction of the delivery device 29, the delivery device can be rotated again to minimize and/or eliminate the gap 14 to facilitate delivery device 29 removal.

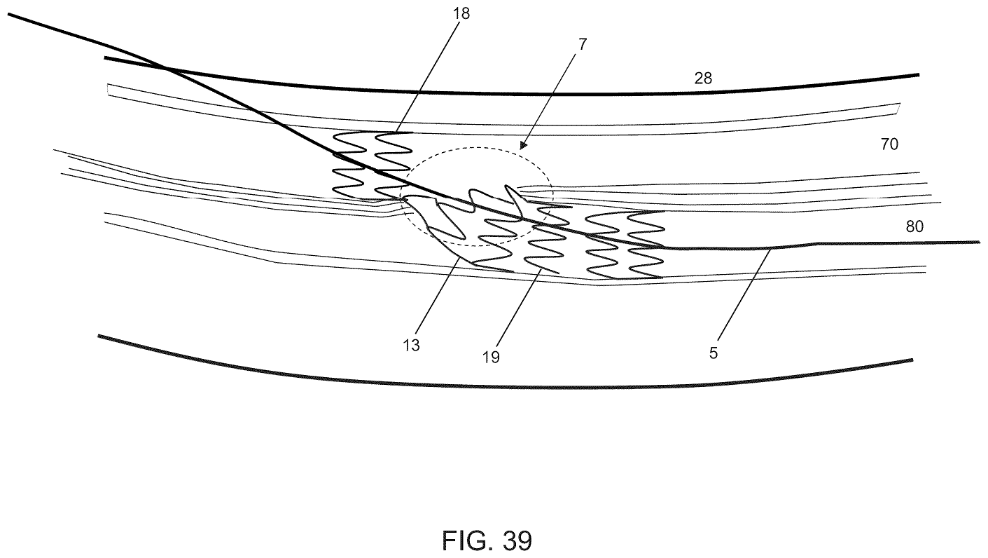

FIG. 39 depicts the complete removal of the delivery device 29 and only the guidewire 5 remaining according to some embodiments. Prior to removal of the guidewire 5, it may be desirable or advantageous to advance a balloon dilation catheter appropriately sized for implant 13 and the vasculature to facilitate complete expansion of the implant 13. In some embodiments with a venous implant segment 19 and an arterial implant segment 18 of different diameters, cross-sectional areas, and/or perimeters, balloon dilation catheters of different sizes may be used to facilitate complete expansion of the venous implant segment 19 and an arterial implant segment 18.

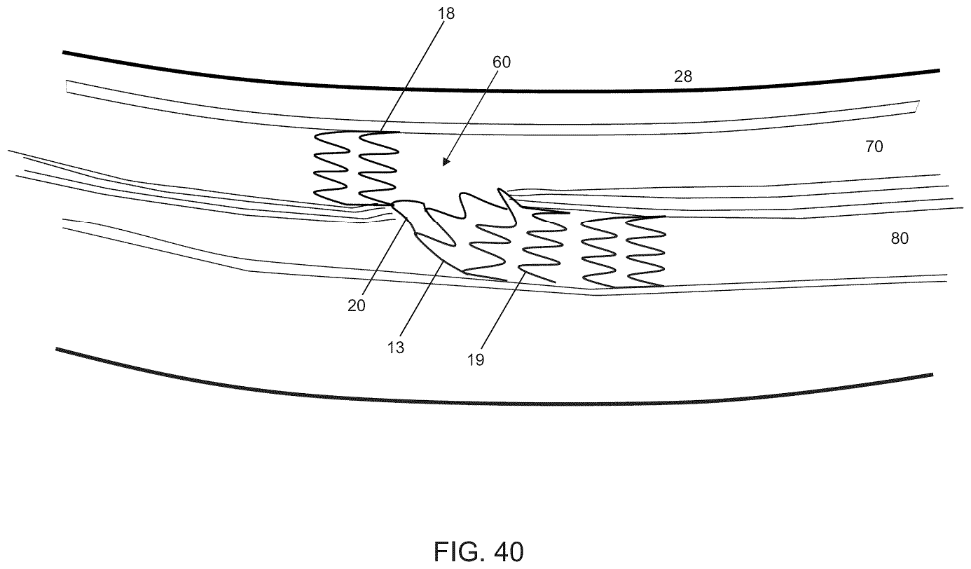

FIG. 40 depicts the completed delivery of the implant 13 with the arterial implant segment 18 in the artery 70, the venous implant segment 19 in the vein 80, and the venous implant segment 19 forming an AVF between the artery 70 and the vein 80. As described herein, the implant 13 may be at least partially anchored in place by any one or more of: (i) the engagement between the wall of the artery 70 and the radially expanded arterial implant segment 18; (ii) the engagement between the wall of the vein 80 and the radially expanded venous implant segment 19; (iii) the engagement between any anatomical structures, such as any portion of the walls of the artery 70 and/or vein 80, and any anchor(s) and/or barb(s) of the implant 13 (not shown in FIG. 40); and (iv) the engagement between the far wall of the artery 70 and the continuous strut/ring 21 (e.g., anastomotic ring) of the implant 13 in embodiments wherein the implant 13 comprises a continuous strut/ring 21. As shown and as described herein, the distal end (as oriented here the proximal end) of the venous implant segment 19 may not obstruct the lumen of artery 70. In some embodiments, the arterial implant segment 18, through connector struts 20, may locate the venous implant segment 19 such that the distal end (as oriented here the proximal end) of the venous implant segment 19 may not obstruct the lumen of artery 70. In embodiments wherein the implant 13 comprises a continuous strut/ring 21, the continuous strut/ring 21 may form a fluidic seal with the far wall of the artery 70. Also shown in FIG. 40 and as described herein, in some embodiments, the venous implant segment 19 (e.g., an axis, such as the longitudinal axis, of the venous implant segment) may be at an angle of between about 0 degrees to about 90 degrees with respect to the arterial implant segment 18 (e.g., an axis, such as the longitudinal axis, of the arterial implant segment). Furthermore, the implant 13 may comprise any one or more of the features, sizes, characteristics, or the like of any of the embodiments of an implant 13 as described herein.

Referring to FIG. 25, prior to implantation of the implant 13, blood flow in the artery 70 may be from right to left, and blood flow within the vein 80 may be from left to right. Referring to FIG. 40, after implantation of the implant 13, blood flow may be as follows: blood flow in the artery 70 may be from right to left and enter the side opening or port 60 (e.g., the side opening or port between the venous implant segment 19 and the arterial implant segment 18) of the implant 13 as shown and (i) flow through the proximal end (as oriented here, distal end) of the arterial implant segment 18 and out the distal end (as oriented here, proximal end) of the arterial implant segment 18 to continue through the artery, and (ii) flow through the distal end (as oriented here, proximal end) of the venous implant segment 19 and out the proximal end (as oriented here, distal end) of the venous implant segment 19 to flow into the vein 80 (and, e.g., flow from left to right in the vein 80 after exiting the venous implant segment 19). In some embodiments and with continued reference to FIG. 40, after implantation of the implant 13 as shown, blood flow through the vein 80 may be at least partially blocked or completely blocked by the venous implant segment 19 (e.g., the blood flow from left to right to the left of the implant 13). Arterial blood may flow through the venous implant segment 19 of the implant 13 due to the pressure differential between the artery 70 and the vein 80. The flow of arterial blood in the vein 80 due to the implant 13 may advantageously cause the vein to increase in at least one of its size (e.g., diameter), thickness, and blood flow rate. For example, the flow of arterial blood in vein 80 due to the implant 13 may advantageously cause the vein 80 to increase in diameter to at least about 4 mm, at least about 5 mm, or at least about 6 mm. In another example, the flow of arterial blood in the vein 80 due to the implant 13 may advantageously cause the blood flow in the vein 80 to be at least about 400 cc/min, at least about 500 cc/min, or at least about 600 cc/min. In some embodiments, the implant 13 may lead the vein 80 to develop into a point of single access for hemodialysis. In some embodiments, the implant 13 may lead the vein 80 to develop a diameter of at least about 6 mm and a blood flow rate of at least about 600 cc/min.

The methods and devices described through FIGS. 25-40 may apply to the creation of an AVF in the vasculature of any relevant area of the human body and for any purpose, including but not limited to the creation of an access point for hemodialysis. For example, the methods and devices described through FIGS. 25-40 may apply to the creation of an AVF between a femoral artery and a femoral vein.

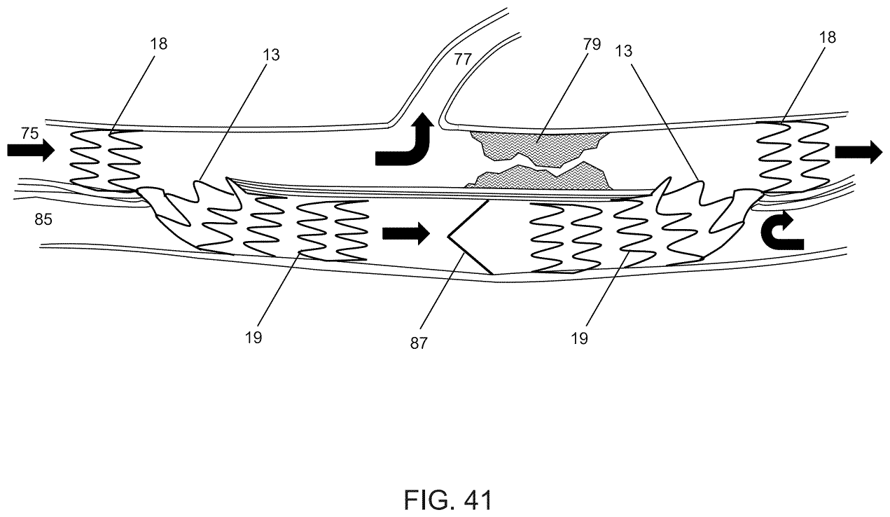

FIG. 41 depicts a method of bypassing a section of an artery with endovascular implants according to some embodiments. Shown is an artery 75 with an arterial branch 77 and an arterial occlusion 79. Also shown is a vein 85 adjacent the artery 75, which may in some embodiments include a vein valve 87. Also shown are two implants 13 implanted such that one (e.g., the left one) creates an AVF between the artery 75 and the vein 85 to the left of (e.g., upstream) of the arterial occlusion 79, and one (e.g., the right one) creates an AVF between the artery 75 and the vein 85 to the right of (e.g., downstream) of the arterial occlusion 79.

Prior to the implantation of the implants 13, blood flow in the artery 75 may be from left to right and may be blocked, substantially blocked, or partially blocked by the arterial occlusion 79, preventing the normal flow of blood through the artery 75. The arterial branch 77 may receive blood flow from the artery, as indicated by the arrow in FIG. 41, as it is positioned to the left (e.g., upstream) of the arterial occlusion 79. Also prior to the implantation of the implants 13, blood flow in the vein 85 may be from right to left, and if a vein valve 87 is present the blood may flow through the vein valve 87 in the same direction.

After the implantation of the implants 13 as shown, arterial blood flow may be as follows: blood flow in the artery 75 may be from left to right and flow through the distal end (per the convention used herein) of the arterial segment 18 of the left implant 13 and out the proximal end (per the convention used herein) of the arterial segment 18 of the left implant 13 and (i) continue to flow through the artery from left to right and either flow through the arterial branch 77 or be blocked by the arterial occlusion 79, and (ii) flow through the distal end (per the convention used herein) of the venous segment 19 and out the proximal end (per the convention used herein) of the venous segment 19 to flow into the vein 85. If a vein valve 87 is positioned in the vein 85 as shown between the left implant 13 and the right implant 13, before or during implantation of the implants a valvulotome or other device may be used to destroy the vein valve 87 so that after implantation of the implants, the arterial blood directed to the vein 85 from the artery 75 by the left implant 13 may continue past the (now destroyed) vein valve 87. Continuing with the arterial blood flow after being directed to the vein 85 by the left implant 13 and any interfering vein valves 87 being destroyed, the arterial blood flow may continue as follows: the arterial blood may continue through the vein 85 after flowing through the venous implant segment 19 of the left implant 13, past any destroyed vein valves 87 if present, flow through the proximal end of the venous implant segment 19 of the right implant 13, flow out of the distal end of the venous implant segment 19 of the right implant 13, flow through the side opening or port 60 of the right implant 13, and (i) flow to the left towards the arterial occlusion 79 before being blocked by the arterial occlusion 79, and (ii) flow to the right through the proximal end of the arterial implant segment 18 of the right implant and out the distal end of the arterial implant segment 18 of the right implant and continue through artery 75. In this way, two implants 13 may be used to restore arterial blood flow in an artery with an arterial occlusion 79. After the implantation of the implants 13 as shown, venous blood flow in the vein 85 may be blocked by the venous implant segment 19 of the right implant 13 as indicated by the return arrow in FIG. 41. The two implants 13 may be covered with a graft material as described herein to facilitate the re-direction of blood flow as discussed relative to FIG. 41.

In continued reference to FIG. 41, in some embodiments the venous implant segment 19 of an implant 13 may traverse a vein valve 87 and obviate the need to destroy the vein valve 87 prior to implantation (e.g., the radial stiffness of the implant may alone be sufficient to open the vein valve 87 and allow for desired blood flow through the vein valve 87). In some embodiments, the delivery device 29 comprises enough axial stiffness to traverse a vein valve 87 and allow for a venous implant segment 19 of the implant 13 to be implanted across the vein valve 87. In some embodiments, the two implants 13 may be overlapped, for example, the venous implant segment 19 of the left implant may be implanted within the venous implant segment 19 of the right implant or vice versa. In some embodiments, the two implants 13 may be spaced apart taking into consideration the vascular anatomy (such as any arterial branches 77) and any arterial occlusions 79. Any of the delivery methods described herein may be used to implant more than one implant 13 as shown in FIG. 41, including the methods described relative to FIGS. 1-15 wherein the delivery device may first access a vein before an artery and the methods described relative to FIGS. 25-40 wherein the delivery device may first access an artery before a vein. Furthermore and as shown in and described relative to FIG. 41, an implant 13 may be used to divert blood flow within the body in multiple ways and is not limited to any one description provided herein. For example, blood flow may enter or exit the distal end of the distal implant segment 18 (i.e., arterial implant segment 18), blood flow may enter or exit the side opening or port 60 between the proximal end of the distal implant segment 18 (i.e., arterial implant segment 18) and the distal end of the proximal implant segment 19 (venous implant segment 19), blood flow may enter or exit the proximal end of the distal implant segment 18 (i.e., arterial implant segment 18), blood flow may enter or exit the distal end of the proximal implant segment 19 (i.e., venous implant segment 19), and blood flow may enter or exit the proximal end of the proximal implant segment 19 (i.e., venous implant segment 19).

As described herein, delivery system 29 may be used interchangeably with delivery device 29. Also as described herein, distal implant segment 18 may be used interchangeably with arterial implant segment 18. Also as described herein, proximal implant segment 19 may be used interchangeably with venous implant segment 19. In some embodiments, the delivery device 29 may be configured to percutaneously deliver the implant 13 into the patient. In some embodiments, the delivery device 29 may be configured to deliver the implant 13 into the patient after a surgical cut down to the AVF location and/or to near the AVF location.

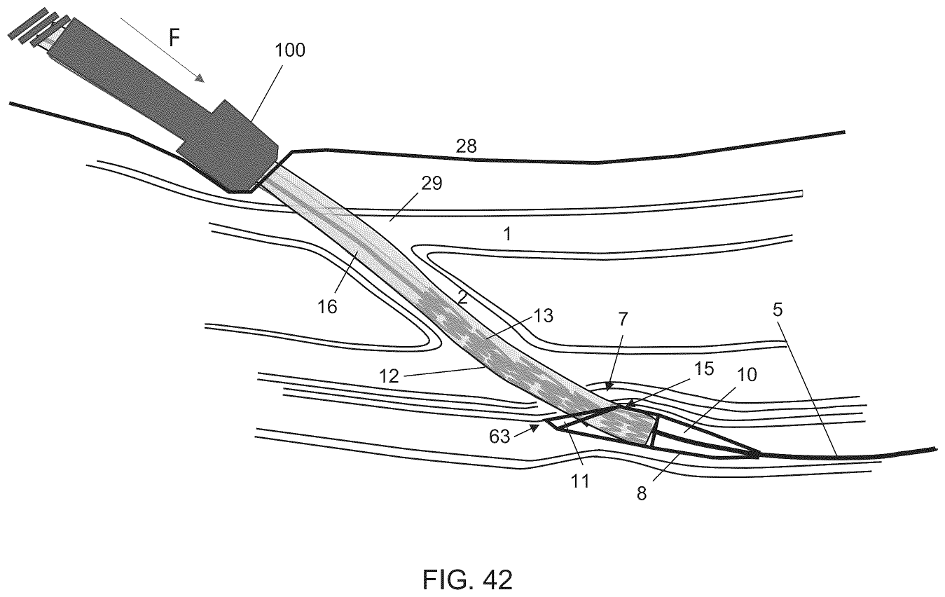

FIG. 42 depicts an embodiment of a counter force mechanism 100 configured to apply a counter force F against the skin/dermal surface 28 of the patient during implantation of implant 13. The counter force mechanism can, for example, apply a counter force to the patient when proximally retracting the outer sheath 12 relative to the nose cone 8 to aid in maintaining engagement of the proximalmost tip 63/proximal tapered end 11 of the nose cone 8 with the wall of the vein or the artery while the proximal implant segment is released from the outer sheath 12. The counter force mechanism 100 can be configured in various ways and can interact with the delivery device 29. For example, the counter force mechanism 100 can comprise a generally tubular body that can be disposed around the outer sheath 12 and apply a force F between a distal end of the handle 23 of a delivery device 29 as described herein (e.g., at or near the handle nose cone 27) and the skin/dermal surface 28 of the patient. In some embodiments, the counter force mechanism 100 can slide over the distal end of the delivery device 29 (e.g., over the nose cone 8 and the outer sheath 12) before implantation of the implant 13. In some cases, the counter force mechanism 100 can comprise two or more pieces that can be joined to one another over the outer sheath 12 at any point during implantation of the implant 13 and/or as needed. In yet other embodiments, the counter force mechanism 100 can include a longitudinal slit/opening sized to accept the outer sheath 12 and/or have a longitudinal slit/opening and be deformable such that the counter force mechanism 100 can be placed over the outer sheath 12 at any point during implantation of the implant 13 and/or as needed. For example, the counter force mechanism 100 can comprise tubing that can deform in the longitudinal direction to provide the counter force F against the skin/dermal surface 28 of the patient when the counter force mechanism 100 is abutted against the distal end of the handle 23 of the delivery device 29 and the patient's skin/dermal surface 28. Such a configuration (e.g., deformable tubing) can allow for customization as needed, such as by cutting the tubing in length as appropriate based on the length of outer sheath 12 that remains outside the patient's body (e.g., sized slightly longer than the exposed outer sheath 12 so that the tubing can deform and apply such a counter force F). As another example, the counter force mechanism 100 can include a spring (e.g., a coil spring) that can provide the counter force F. In some embodiments, the counter force mechanism 100 can include one or more features that can distribute the counter force F against the skin/dermal surface 28, such as a flange and/or protuberance(s) that increase its outward radial size where it meets the skin/dermal surface 28.

As described herein, an implant 13 can have a proximal implant segment 19 and a distal implant segment 18. The proximal implant segment 19 can be defined as the portion of the implant 13 that does not substantially enter or encroach upon a lumen of the vessel in which the distal implant segment 18 is implanted. For example, the distal implant segment 18 can be defined as the portion of the implant 13 that enters and expands within an artery after implantation, and the proximal implant segment 19 can be defined as the portion of the implant 13 that does not substantially enter or encroach upon a lumen of such artery. The proximal implant segment 19 can span from a wall of the vessel in which the distal implant segment 18 is implanted, across any interstitial tissues and/or other vessels, and to another vessel such as a vein. Furthermore, as described herein, an implant 13 can have a side opening 60 between the proximal implant segment 19 and the distal implant segment 18. Continuing with the example above, such side opening 60 can be positioned within the artery and receive blood flow therethrough such that it can flow through the distal implant segment and continue flowing through the artery and/or flow through the proximal implant segment 19 and flow through the vein.

The foregoing description and examples has been set forth merely to illustrate the disclosure and are not intended as being limiting. Each of the disclosed aspects and embodiments of the present disclosure may be considered individually or in combination with other aspects, embodiments, and variations of the disclosure. In addition, unless otherwise specified, none of the steps of the methods of the present disclosure are confined to any particular order of performance. Modifications of the disclosed embodiments incorporating the spirit and substance of the disclosure may occur to persons skilled in the art and such modifications are within the scope of the present disclosure.

Terms of orientation used herein, such as “top,” “bottom,” “horizontal,” “vertical,” “longitudinal,” “lateral,” and “end” are used in the context of the illustrated embodiment. However, the present disclosure should not be limited to the illustrated orientation. Indeed, other orientations are possible and are within the scope of this disclosure. Terms relating to circular shapes as used herein, such as diameter or radius, should be understood not to require perfect circular structures, but rather should be applied to any suitable structure with a cross-sectional region that can be measured from side-to-side. Terms relating to shapes generally, such as “circular” or “cylindrical” or “semi-circular” or “semi-cylindrical” or any related or similar terms, are not required to conform strictly to the mathematical definitions of circles or cylinders or other structures, but can encompass structures that are reasonably close approximations.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

The terms “approximately,” “about,” and “substantially” as used herein represent an amount close to the stated amount that still performs a desired function or achieves a desired result. For example, in some embodiments, as the context may dictate, the terms “approximately”, “about”, and “substantially” may refer to an amount that is within less than or equal to 10% of the stated amount. The term “generally” as used herein represents a value, amount, or characteristic that predominantly includes or tends toward a particular value, amount, or characteristic. As an example, in certain embodiments, as the context may dictate, the term “generally parallel” can refer to something that departs from exactly parallel by less than or equal to 20 degrees.

Where term “about” is utilized before a range of two numerical values, this is intended to include a range between about the first value and about the second value, as well as a range from the first value specified to the second value specified.

Unless otherwise explicitly stated, articles such as “a” or “an” should generally be interpreted to include one or more described items. Accordingly, phrases such as “a device configured to” are intended to include one or more recited devices. Such one or more recited devices can be collectively configured to carry out the stated recitations. For example, “a processor configured to carry out recitations A, B, and C” can include a first processor configured to carry out recitation A working in conjunction with a second processor configured to carry out recitations B and C.

The terms “comprising,” “including,” “having,” and the like are synonymous and are used inclusively, in an open-ended fashion, and do not exclude additional elements, features, acts, operations, and so forth. Likewise, the terms “some,” “certain,” and the like are synonymous and are used in an open-ended fashion. Also, the term “or” is used in its inclusive sense (and not in its exclusive sense) so that when used, for example, to connect a list of elements, the term “or” means one, some, or all of the elements in the list.

Overall, the language of the claims is to be interpreted broadly based on the language employed in the claims. The language of the claims is not to be limited to the non-exclusive embodiments and examples that are illustrated and described in this disclosure, or that are discussed during the prosecution of the application.

Although systems, devices, and methods for endovascular implants and accurate placement thereof have been disclosed in the context of certain embodiments and examples, this disclosure extends beyond the specifically disclosed embodiments to other alternative embodiments and/or uses of the embodiments and certain modifications and equivalents thereof. Various features and aspects of the disclosed embodiments can be combined with or substituted for one another in order to form varying modes of systems, devices and methods for endovascular implants and accurate placement thereof. The scope of this disclosure should not be limited by the particular disclosed embodiments described herein.

Certain features that are described in this disclosure in the context of separate implementations can be implemented in combination in a single implementation. Conversely, various features that are described in the context of a single implementation can be implemented in multiple implementations separately or in any suitable subcombination. Although features may be described herein as acting in certain combinations, one or more features from a claimed combination can, in some cases, be excised from the combination, and the combination may be claimed as any subcombination or variation of any subcombination.

While the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various embodiments described and the appended claims. Further, the disclosure herein of any particular feature, aspect, method, property, characteristic, quality, attribute, element, or the like in connection with an embodiment can be used in all other embodiments set forth herein. Any methods disclosed herein need not be performed in the order recited. Depending on the embodiment, one or more acts, events, or functions of any of the algorithms, methods, or processes described herein can be performed in a different sequence, can be added, merged, or left out altogether (e.g., not all described acts or events are necessary for the practice of the algorithm). In some embodiments, acts or events can be performed concurrently, e.g., through multi-threaded processing, interrupt processing, or multiple processors or processor cores or on other parallel architectures, rather than sequentially. Further, no element, feature, block, or step, or group of elements, features, blocks, or steps, are necessary or indispensable to each embodiment. Additionally, all possible combinations, subcombinations, and rearrangements of systems, methods, features, elements, modules, blocks, and so forth are within the scope of this disclosure. The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed. Thus, some embodiments may be performed using the sequence of operations described herein, while other embodiments may be performed following a different sequence of operations.

Moreover, while operations may be depicted in the drawings or described in the specification in a particular order, such operations need not be performed in the particular order shown or in sequential order, and all operations need not be performed, to achieve the desirable results. Other operations that are not depicted or described can be incorporated in the example methods and processes. For example, one or more additional operations can be performed before, after, simultaneously, or between any of the described operations. Further, the operations may be rearranged or reordered in other implementations. Also, the separation of various system components in the implementations described herein should not be understood as requiring such separation in all implementations, and it should be understood that the described components and systems can generally be integrated together in a single product or packaged into multiple products. Additionally, other implementations are within the scope of this disclosure.

Some embodiments have been described in connection with the accompanying figures. Certain figures are drawn and/or shown to scale, but such scale should not be limiting, since dimensions and proportions other than what are shown are contemplated and are within the scope of the embodiments disclosed herein. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, any methods described herein may be practiced using any device suitable for performing the recited steps.

The methods disclosed herein may include certain actions taken by a practitioner; however, the methods can also include any third-party instruction of those actions, either expressly or by implication. For example, actions such as “positioning an electrode” include “instructing positioning of an electrode.”

In summary, various embodiments and examples of endovascular implants and devices and methods for accurate placement have been disclosed. Although the systems, devices and methods for endovascular implants and accurate placement thereof have been disclosed in the context of those embodiments and examples, this disclosure extends beyond the specifically disclosed embodiments to other alternative embodiments and/or other uses of the embodiments, as well as to certain modifications and equivalents thereof. This disclosure expressly contemplates that various features and aspects of the disclosed embodiments can be combined with, or substituted for, one another. Thus, the scope of this disclosure should not be limited by the particular disclosed embodiments described herein, but should be determined only by a fair reading of the claims that follow.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1 V” includes “1 V.” Phrases preceded by a term such as “substantially” include the recited phrase and should be interpreted based on the circumstances (e.g., as much as reasonably possible under the circumstances). For example, “substantially perpendicular” includes “perpendicular.” Unless stated otherwise, all measurements are at standard conditions including temperature and pressure.

What is claimed is:

1. An implant configured for percutaneous delivery for the creation of an arteriovenous fistula, the implant comprising:
a proximal implant segment comprising a proximal end, a distal end, and an axis extending therethrough;
a distal implant segment connected to the proximal implant segment, the distal implant segment comprising a proximal end, a distal end, and an axis extending therethrough; and
a side opening between the distal end of the proximal implant segment and the proximal end of the distal implant segment;
wherein the implant comprises an hourglass shape.

2. The implant of claim 1, wherein the side opening is configured to allow blood to flow through the side opening (i) in a first direction along the proximal implant segment from the distal end of the proximal implant segment to the proximal end of the proximal implant segment, and (ii) in a second direction along the distal implant segment from the proximal end of the distal implant segment to the distal end of the distal implant segment.

3. The implant of claim 1, wherein the implant comprises an asymmetric hourglass shape.

4. The implant of claim 1, wherein the implant comprises a first longitudinal portion, a second longitudinal portion, a third longitudinal portion, a fourth longitudinal portion, and a fifth longitudinal portion, wherein:
the proximal implant segment comprises, in order from its proximal end to its distal end, the first, second, third and fourth longitudinal portions;
the distal implant segment comprises the fifth longitudinal portion;
the first longitudinal portion has a first diameter;
the third longitudinal portion has a second diameter;
the fifth longitudinal portion has a third diameter;
the second longitudinal portion has a diameter that transitions along its longitudinal length from the first diameter to the second diameter;
the fourth longitudinal portion has a diameter that transitions along its longitudinal length from the second diameter to the third diameter; and
the second diameter is smaller than the first diameter and the third diameter.

5. The implant of claim 4, wherein the third diameter is smaller than the first diameter.

6. The implant of claim 4, wherein the side opening extends from at least a portion of the third longitudinal portion to at least a portion of the fifth longitudinal portion.

7. The implant of claim 1, wherein the proximal implant segment is capable of bending or curving relative to the distal implant segment by at least 180 degrees in a direction away from the side opening.

8. The implant of claim 1, wherein the proximal implant segment comprises a series of rows of struts arranged substantially circumferentially about the axis of the proximal implant segment, wherein adjacent rows of struts of said series of rows of struts of the proximal implant segment are connected to one another by no more than one axially extending strut arranged substantially along the axis of the proximal implant segment.

9. The implant of claim 8, wherein the distal implant segment comprises a series of rows of struts arranged at least partially circumferentially around the axis of the distal implant segment, wherein adjacent rows of struts of said series of rows of struts of the distal implant segment are connected to one another by one or more axially extending struts arranged substantially along the axis of the distal implant segment.

10. The implant of claim 8, wherein a majority or all of the one or more axially extending struts of the proximal implant segment are located on a side of the proximal implant segment aligned with a location of the side opening.

11. The implant of claim 1, wherein the proximal implant segment and the distal implant segment comprise expandable bodies, and wherein the proximal implant segment is oriented at an angle relative to the distal implant segment when the proximal and the distal implant segments are fully expanded in an at rest configuration.

12. The implant of claim 1, wherein the distal end of the proximal implant segment comprises an anastomotic ring.

13. The implant of claim 1, wherein one or both of the proximal implant segment and the distal implant segment comprise one or more anchors configured to anchor said one or both segments against a wall of a vein or an artery.

14. The implant of claim 1, wherein one or both of the proximal implant segment and the distal implant segment are covered with a graft material.

15. The implant of claim 1, further comprising one or more axially extending struts connecting the distal end of the proximal implant segment to the proximal end of the distal implant segment.

16. The implant of claim 1, wherein the implant is configured to allow retrograde blood flow in the artery to (i) flow through the distal end of the distal implant segment and out the proximal end of the distal implant segment, and (ii) flow through the distal end of the proximal implant segment and out the proximal end of the proximal implant segment.

17. An implant configured for percutaneous delivery for the creation of an arteriovenous fistula, the implant comprising:
a proximal implant segment comprising a proximal end, a distal end, and an axis extending therethrough;
a distal implant segment connected to the proximal implant segment, the distal implant segment comprising a proximal end, a distal end, and an axis extending therethrough;
a side opening between the distal end of the proximal implant segment and the proximal end of the distal implant segment; and
a flow restrictor at or adjacent the side opening, the flow restrictor configured to limit blood flow through the proximal implant segment.

18. The implant of claim 17, wherein the implant comprises a first longitudinal portion, a second longitudinal portion, a third longitudinal portion, a fourth longitudinal portion, and a fifth longitudinal portion, wherein:
the proximal implant segment comprises, in order from its proximal end to its distal end, the first, second, third and fourth longitudinal portions;
the distal implant segment comprises the fifth longitudinal portion;
the first longitudinal portion has a first diameter;
the third longitudinal portion has a second diameter;
the fifth longitudinal portion has a third diameter;
the second longitudinal portion has a diameter that transitions along its longitudinal length from the first diameter to the second diameter;
the fourth longitudinal portion has a diameter that transitions along its longitudinal length from the second diameter to the third diameter; and
the second diameter is smaller than the first diameter and the third diameter.

19. The implant of claim 18, wherein the third longitudinal portion comprises the flow restrictor.

20. A method of creating an arteriovenous fistula in a patient, comprising:
delivering an intraluminal implant in a collapsed configuration into the patient, the intraluminal implant comprising:
a proximal implant segment comprising a proximal end and a distal end;
a distal implant segment comprising a proximal end and a distal end,
wherein the proximal end of the distal implant segment is connected to the distal end of the proximal implant segment; and
a side opening between the proximal implant segment and the distal implant segment; and
radially expanding the intraluminal implant between a vein and an artery, wherein the proximal implant segment extends at least partially through the vein, the distal implant segment extends at least partially through the artery, to cause retrograde blood flow in the artery to (i) flow through the distal end of the distal implant segment and out the proximal end of the distal implant segment, and (ii) flow through the distal end of the proximal implant segment and out the proximal end of the proximal implant segment.
